(19) United States
(12) Patent Application Publication
(10) Pub. No.: US 2018/0312868 A1
(43) Pub. Date: Nov. 1, 2018

(54) CHLAMYDOMONAS STRAINS WITH CHLOROPLAST-EXPRESSED CRY PROTEINS FOR BIOLOGICAL CONTROL OF MOSQUITOES THAT TRANSMIT DISEASE

(71) Applicant: BOARD OF REGENTS, THE UNIVERSITY OF TEXAS SYSTEM, Austin, TX (US)

(72) Inventors: David L. Herrin, Cedar Park, TX (US); Seongjoon Kang, Austin, TX (US); OBED W. Odom, Austin, TX (US)

(21) Appl. No.: 15/571,134

(22) PCT Filed: May 2, 2016
(86) PCT No.: PCT/US16/30401

§ 371 Date: Nov. 1, 2017

Related U.S. Application Data
(60) Provisional application No. 62/155,708, filed on May 1, 2015.

(30) Foreign Application Priority Data
Apr. 29, 2016 (US) 15142865

(51) Int. Cl. C12N 15/82; C07K 14/325; A01N 65/03; A01N 63/02

(52) U.S. Cl. CPC C12N 15/8286; C12N 15/8214; G01N 33/56961; A01N 65/03; A01N 63/02; C07K 14/325

(57) ABSTRACT

The present invention relates to producing novel strains of green alga specifically engineered to produce an improved engineered compound over naturally occurring larvicide compound. In particular, genes were isolated and sequenced encoding naturally occurring larvicides produced by Bti (Bacillus thuringiensis subsp. israelensis), e.g. Cry proteins, were redesigned, synthesized, then introduced as heterologous transgenes into strains of Chlamydomonas reinhardtii for producing motile larvicidal-green algae specifically lethal to larvae of mosquitoes and black flies in water systems. Thus green alga (i.e. eukaryote) as motile biocontrol agents are contemplated for use to reduce the number of adult mosquitoes that transmit disease, such as West Nile virus, dengue, encephalitis, and malaria, in addition to reducing the number of adult black flies, in a safe and sustainable manner.

### FIELD OF THE INVENTION

The present invention relates to producing novel strains of green alga specifically engineered to produce an improved engineered compound over naturally occurring larvicide compound. In particular, genes were isolated and sequenced encoding naturally occurring larvicides produced by Bti (Bacillus thuringiensis subsp. israelensis), e.g. Cry proteins, were redesigned, synthesized, then introduced as heterologous transgenes into strains of Chlamydomonas reinhardtii for producing motile larvicidal-green algae specifically lethal to larvae of mosquitoes and black flies in water systems. Thus green algae (i.e. eukaryote) as motile biocontrol agents are contemplated for use to reduce the number of adult mosquitoes that transmit disease, such as West Nile virus, dengue, encephalitis, and malaria, in addition to reducing the number of adult black flies, in a safe and sustainable manner.

### BACKGROUND

Mosquitoes threaten human health by transmitting a number of fatal diseases, including malaria, yellow fever, Dengue, Chikungunya, filariasis, West Nile, and encephalitis. For example, according to the World Health Organization (WHO) there were approximately 207 million cases of malaria and approximately 627,000 deaths in 2012. About 90% of the deaths were in sub-Saharan Africa, and many were children under five years-old (WHO, 2013).

West Nile Virus is a mosquito-borne disease that has become endemic to the U.S., and there is currently no vaccine or treatment for this virus. Most people infected with WNV have no symptoms, but ˜20% experience moderate symptoms for a few days to several weeks. About 1 in 150 infections produce severe symptoms, even death (286 in 2012). According to the CDC, WNV infections are underreported, and they estimated that 86,000-200,000 non-neuroinvasive cases of WNV could have occurred in 2012 (12). The Centers for Disease Control indicated there were 2,374 cases of West Nile Virus in the U.S. in 2013, which resulted in 114 deaths. Recently Texas had 183 cases and 14 deaths (CDC, 2014). Although Dengue is currently not endemic to the United States, it is an emerging disease that infects large numbers of people (50-100 M/yr) in the tropics, and has become endemic in northern Mexico. Mosquito control has so far kept it from becoming entrenched in the United States.

One of the most effective ways to reduce the transmission of these diseases is to control the insect vector (Takken and Knols, 2009). Most mosquito control programs made extensive use of chemical insecticides and they can be very effective. For example, indoor residual spraying and insecticide-treated bednets can reduce malaria cases tremendously (WHO, 2013). However, there are also undesirable effects of chemical insecticides, which include environmental pollution, ecological effects, and human health problems (Margalit, 1989). Also, the evolution of chemically resistant mosquitoes is increasing (Margalit, 1989); in fact, populations of mosquitoes have become resistant to essentially every chemical that was used in the field.

An example of a pesticide family with these issues is pyrethroids, which are chemicals that have been used extensively for indoor residual spraying and in insecticide-treated bednets. Pyrethroids can nonspecifically effect other organisms, including mammals, fishes, and desirable insects, such as honeybees. Pyrethroids are neurotoxins and possible carcinogens in humans (Miyamoto et al., 1995), and pyrethroid resistance among malaria-vector mosquitoes (Anopheles) was reported (Nauen, 2007).

The main goal of using chemical larvicides is to kill or prevent larval development into adult mosquitoes. However, these chemicals are also toxic to fish and other residents of water ecosystems including humans who use these water resources. More specifically, chemical pesticides for mosquito control eventually fail due to the development of resistance in the target larval population. Chemicals also have undesirable effects on non-target organisms, including people, which typically prevent them from being used in densely populated areas. Even with discriminating usage, however, there are growing concerns over long-term low-dose exposure of people to chemical pesticides, especially since their presence was linked to neurodegenerative diseases such as Parkinson's disease.

Therefore, more effective mosquito larvicides are needed for preventing the spread of disease by adult mosquitoes. Additionally, the presence of these new mosquito larvicides in water systems should be safer to humans than those currently being used.

### SUMMARY OF THE INVENTION

The present invention relates to producing novel strains of green algae specifically engineered to produce an improved engineered compound over naturally occurring larvicide compound. In particular, genes were isolated and sequenced encoding naturally occurring larvicides produced by Bti (Bacillus thuringiensis subsp. israelensis), e.g. Cry proteins, were redesigned, synthesized, then introduced as heterologous transgenes into strains of Chlamydomonas reinhardtii for producing motile larvicidal-green algae specifically lethal to larvae of mosquitoes and black flies in water systems. Thus green algae (i.e. eukaryote) as motile biocontrol agents are contemplated for use to reduce the number of adult mosquitoes that transmit disease, such as West Nile virus, dengue, encephalitis, and malaria, in addition to reducing the number of adult black flies, in a safe and sustainable manner.

In one embodiment, the present invention provides a composition comprising a Chlamydomonas chloroplast having a codon-modified cyt1Aa nucleic acid gene sequence in operable combination with a heterologous promoter, wherein said chloroplast expresses a cyt1Aa protoxin. In one embodiment, said codon-modified nucleic acid sequence is SEQ ID NO:18. In one embodiment, said composition further comprises a codon-modified cry11Aa nucleic acid gene sequence, wherein said chloroplast expresses a Cry11Aa protein. In one embodiment, said composition further comprises a codon-modified cry4Aa nucleic acid gene sequence, wherein said chloroplast expresses a Cry4Aa protein. In one embodiment, said composition further comprises a codon-modified gene encoding a starch-binding domain. In one embodiment, said Chlamydomonas chloroplast is part of a Chlamydomonas reinhardtii cell. In one embodiment, said Chlamydomonas reinhardtii is a wild-type organism. In one embodiment, said Chlamydomonas reinhardtii is viable.

In one embodiment, the present invention provides a method comprising introducing a non-native cyt1Aa gene derived from Bacillus thuringiensis sp. israelensis into a Chlamydomonas chloroplast, said cyt1Aa gene comprising a codon-modified nucleic acid sequence, wherein said cyt1Aa gene is in operable combination with a heterologous promoter.

In one embodiment, the present invention provides a method comprising introducing a non-native cyt1Aa gene derived from Bacillus thuringiensis sp. israelensis into a Chlamydomonas chloroplast, said cyt1Aa gene comprising a codon-modified nucleic acid sequence, wherein said cyt1Aa gene is in operable combination with a heterologous promoter, under conditions such that said expressed cyt1Aa gene product provides synergistic activity against mosquito larvae in the presence of a Cry protein. In one embodiment, said Cry protein is selected from the group consisting of Cry4Aa, Cry4Ba, Cry₄Aa₇₀₀, Cry4Ba₆₇₅ and Cry11Aa. In one embodiment, said Chlamydomonas chloroplast further expresses a Cry protoxin non-native gene selected from the group consisting of Cry4Aa, Cry4Ba, Cry4Aa₇₀₀, Cry4Ba₆₇₅ and Cry11Aa. In one embodiment, said promoter is a modified psbD promoter comprising psbD 5′-UTR (psbaₘ).

In one embodiment, the present invention provides a method of treating a body of water comprising mosquito larvae comprising introducing a larvicidal-Chlamydomonas strain, said strain constitutively expressing a cyt1Aa gene product providing synergistic activity against mosquito larvae in the presence of a Cry protein. In one embodiment, said Cry protein is selected from the group consisting of Cry4Aa, Cry4Ba, Cry4Aa₇₀₀, Cry4Ba₆₇₅ and Cry11Aa. In one embodiment, said Chlamydomonas chloroplast further expresses a Cry protoxin non-native gene selected from the group consisting of Cry4Aa, Cry4Ba, Cry4Aa₇₀₀, Cry4Ba₆₇₅ and Cry11Aa. In one embodiment, said Chlamydomonas reinhardtii are toxic to mosquito larvae. In one embodiment, said body of water is treated with a second Chlamydomonas reinhardtii strain, said second strain expressing a Cry protoxin non-native gene selected from the group consisting of Cry4Aa, Cry4Ba, Cry4Aa₇₀₀, Cry4Ba₆₇₅ and Cry11Aa.

In one embodiment, the present invention provides a composition comprising a Chlamydomonas chloroplast having a codon-modified cry11Aa nucleic acid gene sequence in operable combination with a heterologous promoter, wherein said chloroplast expresses a Cry11Aa protoxin. In one embodiment, said nucleic acid sequence is SEQ ID NO:01. In one embodiment, said method further comprises a codon-modified cyt1A nucleic acid gene sequence, wherein said chloroplast expresses a Cyt1A protein. In one embodiment, said method further comprises a codon-modified cry4Aa nucleic acid gene sequence, wherein said chloroplast expresses a Cry4Aa protein. In one embodiment, said method further comprises a codon-modified gene encoding a starch-binding domain. In one embodiment, said Chlamydomonas chloroplast is part of a Chlamydomonas reinhardtii cell. In one embodiment, said Chlamydomonas reinhardtii is a wild-type organism. In one embodiment, said Chlamydomonas reinhardtii is viable.

In one embodiment, the present invention provides a method comprising introducing a non-native cry gene derived from Bacillus thuringiensis sp. israelensis into a Chlamydomonas chloroplast, said cry gene comprising a codon-modified nucleic acid sequence, wherein said cry gene is in operable combination with a heterologous promoter, under conditions such that the cry gene product is expressed constitutively. In one embodiment, said gene sequence comprises a plasmid selected from the group consisting of pCry4A₇₀₀, pCry4B, and pCry11A.

In one embodiment, the present invention provides a method comprising introducing a non-native cry11Aa gene derived from Bacillus thuringiensis sp. israelensis into a Chlamydomonas chloroplast, said cry11Aa gene comprising a codon-modified nucleic acid sequence, wherein said cry11Aa gene is in operable combination with a heterologous promoter, under conditions such that the cry11Aa gene product is expressed constitutively. In one embodiment, said Chlamydomonas chloroplast is a Chlamydomonas reinhardtii chloroplast. In one embodiment, said Chlamydomonas chloroplast is within a Chlamydomonas reinhardtii organism. In one embodiment, said Chlamydomonas reinhardtii is wild-type. In one embodiment, said promoter is a modified psbD promoter comprising psbD 5′-UTR (psbDₘ). In one embodiment, said cry11Aa gene further comprises a downstream region, wherein said downstream region has a 3′ psbA gene untranslated region. In one embodiment, said cry11Aa gene further comprises in operable combination a codon-modified starch binding domain gene, wherein said gene encodes a starch-binding domain. In one embodiment, said Chlamydomonas reinhardtii are viable. In one embodiment, said Chlamydomonas reinhardtii are toxic to mosquito larvae. In one embodiment, said mosquito larvae are A. aegypti larvae. In one embodiment, said gene sequence is SEQ ID NO:01. In one embodiment, said gene sequence is in a vector. In one embodiment, said vector further comprises a codon-modified Cry4Aa sequence. In one embodiment, said vector further comprises a codon-modified Cyt1Aa sequence.

In one embodiment, the present invention provides a method of treating a body of water comprising mosquito larvae (or other larvae) comprising introducing adding a larvicidal-Chlamydomonas strain, said strain expressing a cry11Aa gene product constitutively. In one embodiment, said mosquito larvae comprise A. aegypti larvae. In one embodiment, the body of water is a pond, lake, or stream.

### Definitions

To facilitate an understanding of the present invention, a number of terms and phrases are defined below. The use of the article “a” or “an” is intended to include one or more. As used herein, terms defined in the singular are intended to include those terms defined in the plural and vice versa.

The term “host cell” refers to any cell capable of replicating and/or transcribing and/or translating a heterologous gene, such as a single cell or multiple cell organism.

As used herein, the term “green algae” refers to a diverse group of algae (singular: green alga), with more than 7000 species growing in a variety of comprising chlorophyll, which they use to capture light energy to fuel the manufacture of sugars, but unlike plants they are primarily aquatic. In other words, green algae are aquatic organisms that thrive on sunlight and carbon dioxide (or bicarbonate).

As used herein, the term “Chlamydomonas” in general refers to a single cell eukaryote organism within a genus of 500+ different species of unicellular photosynthetic green algae or “microplant” which often expresses two flagella for motility, along with a single chloroplast organelle which occupies the greater part of the cell. Chlamydomonas species are found in soil, fresh water, oceans, snow on mountaintops, etc., including the species Chlamydomonas reinhardtii. Chlamydomonas grow well heterotrophically (in darkness), and grows best when provided both light and organic acids (acetate), thus frequently found growing (viable) in polluted environments (27) including environments containing insect larvae. Chlamydomonas are used for development of strains for use in bioremediation.

As used herein, the term “Chlamydomonas reinhardtii” refers to a species of Chlamydomonas, including but not limited to varieties (var.) intermedia R. H. Chodat C, Chlamydomonas reinhardtii f. basimaculata Compère C, Chlamydomonas reinhardtii var. minor G. Nygaard C, Chlamydomonas reinhardtii var. lateovalis (Brabez) L. Péterfi C, Chlamydomonas reinhardtii P. A. Dangeard C-type, and engineered inducible strains and laboratory strains, such as described herein, etc.

As used herein, the term “wild type” or “wild-type” in reference to Chlamydomonas organisms refers to organisms found in nature that were not modified or engineered. Wild type in reference to a strain refers to Chlamydomonas organisms that were isolated from nature and grown or maintained in a laboratory (an artificial environment).

As used herein, the term “strains” in reference to Chlamydomonas organisms refers to organisms within the same species or subspecies having different functions or genetics, such that a transgenic Chlamydomonas reinhardtii expressing a cyr11Aa gene is a different strain than an otherwise identical strain (such as a wild type strain) that is does not contain a cyr11Aa transgene. A “larvicidal strain” for mosquitoes, such as larvicidal-Chlamydomonas of the present inventions refers to an engineered strain that when used as a food source (i.e. edible larvicidal-Chlamydomonas) has the capability to delay the development of or kill mosquito larvae.

As used herein, the term “edible” or “digestible” refers to an organism or substance suitable for to use for food. As one example, mosquito larvae and other organisms eat Chlamydomonas species as a source of nutrition, thus Chlamydomonas species are edible.

As used herein, the term “viable” refers to an organism that is capable of growing and living under certain environmental conditions, as one example when the growth rate shows an increase rather than a decrease in the number of organisms when grown under certain laboratory environments, for example, when growing and living in a simple medium of inorganic salts, using photosynthesis to provide energy.

As used herein, the term “Bacillus thuringiensis” or “Bt” refers to a group of aerobic, Gram-positive bacterium found in: the soil, gut of caterpillars of various types of moths and butterflies, as well on leaf surfaces, aquatic environments, animal feces, insect-rich environments, flour, grain-storage facilities, etc. Many Bt strains produce crystal proteins (proteinaceous inclusions, also called δ-endotoxins), from plasmid-encoded cry genes that have insecticidal action. These crystal proteins are a mixture of different protoxins with different Bt strains having different relative amounts of the protoxins, each of which is active against a subset of insect larvae.

As used herein, the term “Bti” or “Bacillus thuringiensis subsp. israelensis” refers to a specific subspecies of Bt bacteria. During sporulation, Bti produces a parasporal body (PB) that contains larvicidal activity toward Dipterans, including mosquitoes (Anopheles, Aedes, and Culex families) and black flies. Bti was thus different from the known subspecies of Bacillus thuringiensis, which were toxic mostly to lepidopteran insects (Margalit, 1989). The parasporal body (PB) of Bti H-14 has a crystal-like structure and contains two types of larvicidal proteins: crystal (Cry) proteins and cytolysins (Cyt) (FIG. 1).

There are at least 3 major “Cry” or “CRY” or “crystal” larvicidal “Bti” proteins (polypeptides) termed Cry4Aa, Cry4Ba, Cry11Aa with molecular weights (from the predicted sequences) of 134, 128, and 72 respectively (Frankenhuyzen, 2009; Poopathi and Abidha, 2010; Bravo et al., 2011; Laurence et al., 2011). Further, a “Bti” cytolysin “Cytolytic” or” CYT″ or “Cyt” or “CRT” protein refers to a protein that can lyse a cell, for example Cyt1Aa, around 28 kDa. Cry and Cyt protoxin encoding genes are found on a 128-kb plasmid called pBtoxis (Berry et al., 2002), and the genes' sizes are 3543 bp (1180 amino acids) for Cry4Aa, 3408 bp (1136 amino acids) for Cry4Ba, 1929 bp (643 amino acids) for Cry11Aa, and 744 bp (248 amino acids) for Cyt1Aa (Ben-Dov, 2014). Thus, the Cry genes are large (Cry11Aa) to very large (Cry4Aa and Cry4Ba), and the Cyt (or crt) gene is a common size for bacterial proteins. Upon sporulation of the bacterium, the toxin genes on pBtoxis are expressed and the resulting proteins are assembled into the crystal-like PB (Ibarra and Federici, 1986). Cry4Aa+Cry4Ba, Cry11Aa, and Cyt1Aa are found as 3 distinct sub-inclusion bodies that are surrounded by a lamellar-like envelope (FIG. 1) (Federici et al., 2003). In addition, Cry10Aa, Cyt2Ba, and Cyt1Ca are minor toxins found in the PB (Ben-Dov, 2014). When sporulation is complete, the crystal endotoxin (PB) and the endospore are released from the bacteria cell. Ingestion of the crystals by mosquito and fly larvae can result in growth inhibition and death, with the effective toxicity being determined by a number of factors. Additional Cry molecules are shown in FIG. 2.

As used herein, the term “cry” or “CRY” or “Cry” or “crystal” in general refers to a gene or protein within a large family of crystalline protoxins, such as produced by a Bacillus thuringiensis bacterium, varieties, subspecies, strains, etc., thereof. As an example, CRY proteins of B. thuringiensis sp. were classified based on size, homology of the amino acid sequence, and pathogenicity (Hate and Whiteley, 1988, Crickmore et al., 1998). Based on the size of the protoxins, Cry proteins were generally grouped as: ˜130 kDa and ˜70 kDa (Hate and Whiteley, 1989). Cry4Aa and Cry4Ba belong to the former, while Cry11Aa belongs to the latter group. The 130-kDa proteins contain a highly conserved C-terminal region rich in cysteine, some of which are involved in disulfide bonds and formation of the inclusion body (Hate and Whiteley, 1989); however, the N-terminal region confers toxicity. The 70-kDa group does not have the C-terminal region, but these proteins have structural similarities with the N-terminal region of the 130-kDa group proteins (FIG. 2) (Jurat-Fuentes and Jackson, 2012).

Additionally, as used herein, the term “cry” or “CRY” or “crystal” depending upon its context, as an example, Cry11Aa, may also refer to a novel codon modified synthetic gene or its expressed protein as described herein. Such that a cry gene of the present inventions may be “derived from” a sequence copied from a naturally occurring sequence. For example, a novel cry gene of the present inventions that is a non-native, codon-modified nucleic acid sequence was “derived from” a Bacillus thuringiensis sp. (i.e. subspecies) israelensis.

As used herein, the term “derived” in reference to “derived from” a gene sequence of the present inventions refers to a codon modified sequence having at least 76%, 77%, or 78% or greater (80%, 90% or more) identity to a native sequence (see Table 2), that is used for designing an encoding DNA sequence. It is preferably not less than 65% identical from the sequence from which it is derived. Thus a novel encoding DNA sequence, such as used in the present inventions for expressing Cry11, is derived from (or reverse engineered from) an amino acid sequence is different than a naturally found encoding DNA sequence.

As used herein, the term “engineered” refers in general to an artificial process of manipulating nucleic acid sequences, such as by ligating (such as by using a ligase enzyme) two or more isolated nucleic acids sequences to each other, or synthesizing an artificial gene, or making a product, such as a transgenic Chlamydomonas organism.

As used herein, the term “produce” in reference to producing a larvicide refers to the capability of an engineered Chlamydomonas to transcribe a Cry encoding DNA sequence then translating it into a Cry protein so that the transgenic Chlamydomonas produces a larvicide, for example, a “larvicide-producing algae” or “larvicide-producing Chlamydomonas.

As used herein, the term “larvicide” refers to a compound that targets the larval life stage of an insect such that the compound either kills (causes death of larvae) or inhibits the development of immature larvae into adult insects, thus “toxic” to the larval form of an insect. A larvicide may also be referred to as a “control agent.”

As used herein, the term “larva” and “larvae” refer to immature forms of insects.

As used herein, “pathogen” refers a biological agent that causes a disease state (e.g., infection, illness, death, etc.) in a host. “Pathogens” include, but are not limited to, viruses, bacteria, archaea, fungi, protozoans, mycoplasma, parasitic organisms and insects.

As used herein, the term “disease” refers to human and animal illness or death caused by pathogens, diseases include but are not limited to West Nile virus, dengue, encephalitis, malaria, filarial disease, i.e. a parasitic disease that is caused by thread-like roundworms belonging to the Filarioidea type. Blood-feeding black flies and mosquitoes spread filarial disease.

As used herein, the term “carrier” or “vector” in reference to a disease or pathogen refers to an insect or other organism that harbors a pathogen, such as mosquitoes harboring Plasmodium species that are capable of causing malaria in a subject when the adult mosquito, carrying the disease causing Plasmodium, bites the subject.

As used herein, the term “transmit” refers to the movement of a pathogen to a subject via a carrier organism.

As used herein, the term “subject” refers to any mammal, preferably a human patient, livestock, or domestic pet.

As used herein, the term “mosquito” refers to a midge-like fly in the Culicidae family. Although the majority of species are not harmful, mosquito-borne diseases cause millions of deaths worldwide every year. In particular, the Anopheles species is known to carry malarial pathogens. Mosquitoes also transmit pathogens for diseases such as filariasis (also called elephantiasis), encephalitis, and the West Nile virus. The Asian tiger mosquito carries pathogens causing yellow fever, dengue, and encephalitis. In addition to humans, mosquitoes feed upon and pass on pathogens to subjects including but not limited to horses, cattle, and birds. Organisms including but not limited to dragonflies, bats, birds, spiders, etc in turn eat adult mosquitoes.

As used herein, the term “mosquito larvae” refers to immature mosquitoes living in water systems (aquatic) mainly slow moving streams, ponds and stagnant water, in general having a soft body, a hard head and a breathing tube, or siphon, at the tip of the abdomen, feeding upon algae and bacteria.

As used herein, the term “black fly” in reference to a small insect refers to a member of the family Simuliidae of the Culicomorpha infraorder which are biting pests of wildlife, livestock, poultry, and humans. Alternatively called buffalo gnat, turkey gnat, or white socks, black flies transmit (i.e. as carriers or vectors) filarial disease (for example, onchocerciasis (river blindness)), Additionally, reactions to black fly bites in humans are collectively known as “black fly fever” including headache, nausea, fever, and swollen lymph nodes in the neck. Black flies are capable of transmitting a number of different disease agents to livestock, including protozoa and nematode worms, when numerous enough, black flies have caused suffocation by crawling into the nose and throat of pastured animals. Black flies are known to cause exsanguinations (death due to blood loss) from extreme rates of biting. Saliva injected by biting black flies can cause a condition known as “toxic shock” in livestock and poultry, which may result in death. Non-biting black fly species fly around the head and may crawl into the ears, eyes, nose, or mouth, causing extreme annoyance to animals or people engaged in outdoor activities.

As used herein, the term “black fly larvae” refers to immature black flies living in water systems (aquatic) mainly fast moving streams and ponds.

As used herein, the term “water system” refers to a particular water source, such as a “water supply system” or “water supply network” of natural, such as a river, its branches and underground tributaries or other water connections, or engineered hydrologic and hydraulic components that provide a water supply. Examples include but are not limited to, a lake, pond, river, creek, irrigation systems, rainwater collection units, sewer systems, enclosed water containers, hydroponic systems, etc.

As used herein, “safe” in reference to environmental activity refers to a condition of exposure under which there is a practical certainty that no harm will result to the ecosystem, such as no harm to the surrounding ground, air, and water, including ground water, surface water, drainage water and any bodies of water into where drainage water flows.

As used herein, “sustainable” as in “sustainable manner” in reference to “ecological sustainability” or “environmental sustainability” refers to current methods of ecosystem maintenance, including components and functions, in order to provide safe and healthy ecosystems for future generations of plants, fish, reptiles, mammals, and microbial communities.

As used herein, “ecology” refers to a relationship of living organisms to one another and their environment, or the study of such relationships.

As used herein, “ecosystem” refers to an interacting system of a biological community, including but not limited to plants, fish, reptiles, mammals, and microbial communities, and its non-living environmental surroundings, such as soil, water, and air.

As used herein, “desired benefit” in relation to humans, refers to any effect that confers a benefit to humans and animals.

The terms “protein” and “polypeptide” refer to compounds comprising amino acids joined via peptide bonds and are used interchangeably.

As used herein, “amino acid sequence” refers to an amino acid sequence of a protein molecule. “Amino acid sequence” and like terms, such as “polypeptide” or “protein,” are not meant to limit the amino acid sequence to the complete, native amino acid sequence associated with the recited protein molecule. Furthermore, an “amino acid sequence” can be deduced from the nucleic acid sequence encoding the protein.

The term “compartments” or “organelles” in reference to a plant cell is used in its broadest sense. The term includes but is not limited to, the endoplasmic reticulum, Golgi apparatus, trans Golgi network, plastids including chloroplasts, proplastids, and leucoplasts, sarcoplasmic reticulum, glyoxysomes, mitochondrial, chloroplast, and nuclear membranes, and the like.

The term “chloroplast” or “plastid” or “thylakoid” refers to a specialized organelle, including its membrane, found in plant and algal cells for conducting photosynthesis. A chloroplast has photosynthetic pigments called chlorophyll which captures energy from sunlight then uses this energy to make sugars and other compounds and stores it in energy storage molecules, such as ATP and NADPH. A chloroplast contains DNA as a chloropalst genome comprising DNA molecules, often in association with the chloroplast membrane.

The term “codon” or “triplet” refers to a nucleotide sequence of three nucleotides as three adjacent (attached to each other within a gene) deoxyribose nucleic acids or three adjacent ribose nucleic acids (attached to each other within a transcribed RNA) that encode a specific amino acid or a control signal during transcription or translation, respectively. Several condons may represent the same amino acid, in other words “degenerate codons” or “synonymous codons.” “Degeneracy” in reference to the genetic code means that one amino acid can be encoded by several codons. As one example, CAT or CAC (DNA) and CAC or CAU (RNA) encode or represent the amino acid Histidine. In other words, CAT and CAC are “synonymous.” Further, each particular organisum may not use the available codons randomly, but may show a certain preference for having or “using” particular codons for the same amino acid, such that each individual genome may use a preferred set of codons.

The term “codon usage” or “codon bias” or “codon preference” or “codon usage preference” refers to frequencies of codons that code for the same amino acid (i.e. synonymous codons) found in genes expressed by a particular organisum, such as E. coli, or within a genome, such as genes expressed within a chloroplast genome. a statistical property of DNA sequences that encode proteins. For example, analysis of a chloroplast genome shows a bias or preference for using certain codons by genes expressed within a chloroplast, which may be different than found for certain genes expressed within a nuclear genome. Codon usage may also vary from organism to organism, such that codons preferred by E. coli may be different than in Chlamydomonas. In other words, codon preference refers to a phenomenon where specific codons are used more often than other synonymous codons during translation, such that the codon usage preference correlates with the abundance of tRNAs for a given amino acid, i.e. more frequent codons may have more abundant corresponding tRNAs in the host organism.

The term “codon-modified” refers to changing at least one nucleotide for another nucleotide within a triplet sequence, often in the third position, resulting in the translation of a protein containing the same amino acid for that position (such as changing a CAT to CAC).

The term “codon adapted” or “codon optimization” refers to artificially changing at least one nucleotide within a codon of a heterologous gene to increase the frequency of codons used from weakly expressed genes to that used by highly expressed genes, or at least one nucleotide within a codon of a bacteria gene to increase the frequency of codons used when the bacteria gene sequence is used as a heterologous gene to codon usage of a chloroplast genome, see examples for Cry11Aa in the shaded areas of FIG. 6A. A Codon Adaptation Index (CAI) provides values for the original sequence compared to an adapted sequence. Codons are adapted in a heterologous gene for a contemplative increase in heterologous gene trascription and translation with the contemplative purpose of increasing heterologous protein production, i.e. increasing Cry11Aa protein (protoxin) production. However, as shown herein, codon adaptation does not guarentee protein expression, see for example, Cry4B as described herein.

The term “gene optimization” refers to selecting codons, such as from a codon usage table for a particular host organism, for changing at least one codon in a heterologous gene encoding a given protein sequence for the purpose of increasing the expression efficiency and thus increasing the amount of protein produced by the optimized heterologous gene expressed by that organism. As one example, The Kazusa codon usage database contains codon usage tables created from complete genomes for organisms found in Genbank (NCBI).

The term “non-native” or “modified” in reference to a DNA or RNA sequence refers to a sequence of nuclotides (eiher DAN or RNA) that is not found in a native, unmodified (unengineered) genome of an organisum. For example, the modified a psbDₘ promoter region of the present inventions does not match a psbaₘ promoter region.

The term “polynucleotide” refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “transformation” as used herein refers to introduction of an inheritable alteration/mutation to eukaryote (e.g. Chlamydomonas) and prokaryotic cells (e.g. E. coli) from the uptake, incorporation, or expression of foreign DNA. Transformation may be accomplished by many means known in the art. For example, chemically induced, microinjection, protoplast fusion, electroporation, lipofection, viral infection etc. Also see transfection.

As used herein, the term “transfection” or “introduced” in relation to a host refers to the introduction of foreign DNA into host cells (e.g. Chlamydomonas organisms). Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment, gene gun, etc.) and the like.

As used herein, the term “eukaryote” refers to an organism having a nucleus and other membrane bound structures.

The term “expression vector” or “vector” as used herein refers to a recombinant DNA molecule containing a desired coding sequence and appropriate nucleic acid sequences necessary for the expression (i.e., transcription and/or translation) of the operably linked coding sequence in a particular host organism. The most preferred vector as used herein, is the bacterial artificial chromosome vector but other expression vectors are exemplified by, but not limited to, bacterial plasmid, phagemid, shuttle vector, cosmid, virus, chromosome, mitochondrial DNA, plastid DNA, and nucleic acid fragment. Nucleic acid sequences used for expression in prokaryotes include a promoter, optionally an operator sequence, a ribosome-binding site and possibly other sequences. Eukaryotic cells are known to utilize promoters, enhancers, and termination and polyadenylation signals.

The terms “in operable combination”, “in operable order” and “operably linked” as used herein refer to the linkage of nucleic acid sequences in such a manner that a nucleic acid sequence (such as a promoter) is capable of directing the transcription of a given gene and/or the synthesis of a desired protein.

The term “promoter” as used herein refers to a nucleotide sequence in DNA to which RNA polymerase binds to begin transcription. A promoter may be inducible or constitutive. One example of a promoter is a psbD promoter for a chloroplast psbD gene, which encodes the photosystem II reaction center polypeptide D2.

The term “control regions” or “regulatory elements” as used herein in reference to gene transcription refers to genes such as promoters and enhancers, whose presence may increase or decrease transcription, for examples, a psbA regulatory element, used herein, from the 3′ untranslated region of a psbA gene, which codes for the D1 polypeptide of the photosystem II reaction center complex in chloroplasts.

The term “gene” refers to a nucleic acid (e.g., DNA or RNA) sequence, such as DNA that comprises coding sequences necessary for the production of RNA, including mRNA further encoding a polypeptide (e.g., a protoxin). A functional polypeptide can be encoded by a full-length coding sequence or by any portion of the coding sequence as long as the desired activity or functional properties (e.g., toxicity, enzymatic activity, ligand binding, signal transduction, etc.) of the polypeptide are retained.

The term “gene” also encompasses the coding regions of a structural gene and includes untranslated sequences located adjacent to the coding region on either or both of the 5′ and 3′ ends, and intervening untranslated regions, such that the term “gene” corresponds to the length of the entire length of DNA involved with expression of a full-length mRNA. The sequences that are located 5′ of the coding region, which sometimes are present on the mRNA, are referred to as upstream or 5′ non-translated sequences (UTR). The untranslated (UTR) sequences which are located 3′ or downstream of the coding region, which sometimes are present on the mRNA, are referred to as 3′ non-translated sequences. The term “gene” encompasses both cDNA, genomic DNA and synthetic DNA. A genomic form or clone (copy) of a gene in a genome often contains the coding region interrupted with non-coding sequences termed “introns” or “intervening regions” or “intervening sequences.” Introns are segments of a gene that may contain regulatory elements such as enhancers. Introns are removed or “spliced out” from a primary RNA transcript; introns therefore are absent in the messenger RNA (mRNA) transcript. The mRNA functions during translation to specify the sequence or order of amino acids in a nascent polypeptide.

In addition to containing introns, a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The term “heterologous” in reference to a nucleic acid sequence refers to a piece of DNA that is not in its natural environment (i.e., has been altered by the hand of man). For example, a heterologous nucleic acid sequence includes a piece of DNA from one species introduced into another species, such as promoters and enhancers used in the present inventions, including but not limited to regulatory regions such as psbD and psbA.

The term “heterologous gene” refers to a gene encoding a factor that is not in its natural environment (i.e., has been altered by the hand of man). For example, a heterologous gene includes a gene from one species introduced into another species. A heterologous gene also includes a gene that is synthetically reversed engineered from a protein (amino acid) sequence or a gene native to an organism that has been altered in some way (e.g., mutated, added in multiple copies, linked to a non-native promoter or enhancer sequence, etc.). Heterologous genes may comprise bacteria gene sequences that comprise cDNA forms of a bacteria gene (such that at least some of the intervening DNA sequences are removed); the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with that gene for the protein encoded by the heterologous gene or with gene sequences in the chromosome, or are associated with portions of the chromosome not found in nature (e.g., genes expressed in loci where the gene is not normally expressed).

The term “marker” as used herein refers to a protein and its encoding gene which encodes a protein used for identifying expressed proteins or an enzyme having an activity that confers resistance to an antibiotic (ampicillin, kanamycin, chloramphenicol, zeocin, tetracycline, etc.) drug, or digestion of an indicator such as X-gal, upon the cell in which the marker for selection is expressed, or which confers expression of a trait which can be detected (e.g., luminescence or fluorescence). Examples are Flag, beta-galactosidase, green fluorescent protein (GFP), luciferase, xanthine phosphoribosyltransferase, antibiotic resistance, etc.

The term “portion” when used in reference to a gene refers to fragments of that gene or in reference to a protein, a fragment of that protein. The fragments may range in size from a few nucleotides (or amino acids) to the entire gene sequence (or protein) minus one nucleotide. Thus, “an amino acid comprising at least a portion of a protein” may comprise fragments of the protein or the entire protein.

The term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “an oligonucleotide having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

### BRIEF DESCRIPTION OF THE DRAWINGS

FIG. 1 shows exemplary (A) Bacillus thuringiensis subsp. israelensis (Bti) containing spore (Sp) and parasporal body (PB). (B) A parasporal body of Bti containing 3 subinclusion bodies that are composed of Cry4A+Cry4B, Cry11A, and Cyt1A, respectively. (Adapted from Federici et al., 2003).

FIG. 2 shows exemplary Cry structures with conserved blocks of amino acids. Activated Cry toxins consist of three Domains (I-III), which encompass conserved blocks 1-5. Each Cry protein has at least one conserved block. The darker color of the block indicates a higher degree of homology. Var, Variant; alt, alternate. Adapted from Schnepf et al. (1998). Functionally, Domain I is involved in inserting into the membrane and forming a pore, while Domains II and III are responsible for receptor binding and toxin specificity (de Maagd et al., 2001). Domain I is composed of 5-7 alpha-helices (Xu et al., 2014), with a central hydrophobic helix (α5) surrounded by amphipathic helices (Boonserm et al., 2006; Leetachewa et al., 2006). Domain II is comprised of 3 antiparallel β-sheets (β-prism) in a “greek key” motif, with a hydrophobic core helix and three apical loops (Xu et al., 2014). Domain II has the most variable sequence, with the lengths and sequences of the exposed apical loops showing high divergence (Boonserm et al., 2005).

FIG. 3 shows exemplary three-dimensional structures of activated Bti toxins Cry4Aa, Cry4Ba, Cry11Aa and Cyt1Aa by X-Ray crystallography (Boonserm et al., 2005; Boonserm et al., 2006; Cohen et al., 2011). These 3 Cry proteins have a three-domain structure: Domain I is the α-helix bundle; Domain II is called the β-prism, and Domain III is the β-sandwich. Functionally, the loops in Domain II are involved in interactions with the receptors, and determine much of the specificity. For example, Loop 2 in Domain II of Cry4Aa is essential for toxicity against Culex pipiens (Howlader et al., 2009). Abdullah et al. (2003) replaced Loop 3 of Cry4Ba with Loop 3 of Cry4Aa and increased the toxicity of Cry4Ba against Culex; they also showed that Loops 1 and 2 are determinants of Cry4Ba activity against Aedes and Anopheles. In Cry11Aa, Loop α-8 is an epitope that interacts with gut receptors in A. aegypti (Fernandez et al., 2005); Cry11Aa-receptor interactions also seem to involve β-4 and Loop 3 (Fernandez et al., 2005). Domain III consists of two antiparallel β-sheets (β-sandwich) in a jelly roll” topology (Soberón et al., 2010; Xu et al., 2014). It is the most conserved region, with 3 conserved blocks (FIG. 2). This domain was suggested to participate in membrane permeability or receptor binding and insect specificity (de Maagd et al., 2001). Cyt1A is one-domain protein comprised of two α-helix layers surrounding a β-sheet (Cohen et al., 2011; Bravo et al., 2011). Upon activation, α-helices A, B, C and D stay outside the membrane while β-strands 5, 6 and 7 enter the membrane forming a pore (Soberon, et al., 2013). Stable folding and crystallization of Cyt1Aa in the PB in vivo is aided by P20, a chaperone located in the Cry11Aa operon (Visick and Whiteley, 1991; Dervyn et al., 1995). Structures (A), (B), and (D) were determined from X-Ray crystallography, whereas structure (C) is an in silico model predicted by homology modeling with the three-dimensional structure of Cry2Aa. Adapted from Angsuthanasombat et al. (2004), Fernandez et al. (2005), and Cohen et al. (2011).

FIG. 4 shows an exemplary schematic diagram of a Chlamydomonas cell based on transmission electron microscopic pictures. The cell has a cup-shaped chloroplast with a pyrenoid near the base, surrounded by starch granules, and an eyespot with carotenoids. The cell also has a nucleus, mitochondria, and two anterior flagella. Adapted from Merchant et al. (2007).

FIG. 5 shows an exemplary inducible chloroplast gene expression system used herein for expressing transgenic Cry genes. For example, in the inducible Ind41_18 host strain of Chlamydomonas, expression of the Cry constructs is controlled by the host nuclear Cyc6:Nac2 gene, which is in turn controlled by Cu²⁺ levels. The presence of Cu²⁺ inhibits the expression of the Nac2 gene, which causes repression of the Cry gene flanked by psbD 5′ UTR. When Cu²⁺ is removed, the NAC2 protein is made and binds to the psbD 5′-UTR of the chimeric Cry mRNA, stabilizing it. (The diagram was adapted from Ramundo et al., 2013).

FIG. 6 shows exemplary schematics of modified codon usage related to Cry gene constructs of the present inventions compared to a native Bti sequence. (A) shows an exemplary representative comparison between a native Bti toxin sequence (Bti) and a codon-adapted (modified) novel (ca) toxin sequence; this part of Cry11Aa corresponds to amino acids 1-36. Nucleotides that were changed are shaded. (B) shows exemplary synthetic codon-optimized (modified) Cry genes: Cry4Aa₇₀₀, Cry4Ba, and Cry11Aa. Genes were designed using a combination of the native toxin amino acid sequences, the program Optimizer, and a codon-usage table based on highly expressed Chlamydomonas chloroplast genes. After optimization, the codon adaptive index (CAI) for each gene increased from ˜0.5 to 1. The Flag epitope tag for antibody-detection was added to the C-terminus of each of these three genes. Integrated DNA Technologies synthesized these novel genes for providing synthetic template DNA sequences for use with the present inventions.

FIG. 7 shows exemplary diagrams of Cry gene constructs of the present inventions and the site of integration in the chloroplast genome of Ind41_18. Expression of the Cry genes is controlled by a modified psbD promoter/5′-UTR (psbDₘ) and 3′ region from psbA. The locations of primers used for PCR screening of the transformants (FIG. 8) are indicated. Note that primers 864 and 865 are located upstream and downstream, respectively, of the integration site in CpDNA. Some parts of the diagram are not drawn to scale.

FIG. 8 shows exemplary results from PCR analysis of chloroplast transformants. Diagrams of the primers and expected sizes of the respective PCR products are indicated above the agarose gels, whose fluorescence images were inverted. (A) Analysis of two Cry4Aa₇₀₀ transformants. Wild-type DNA was used as the positive control for the integration site as it is identical to Ind41_18 in this region. pCry4A is the plasmid that was shot into the chloroplast. The other lanes contained size markers (M) and a reaction with no DNA (−) as a negative control. (B) Analysis of a Cry4Ba transformant. The other lanes were similar to (A). (C) Analysis of two Cry11Aa transformants. Lanes WT, M and (−) were similar to (A), and lane pCry11A was the plasmid that was shot into the cells.

FIG. 9 shows exemplary results from Western blot analysis of Cry proteins expressed in the chloroplast with the inducible system. (A) The Cry4A 700 (4A) and Cry11A (11A) transformants were grown under Uninduced (+Cu 2+) and Induced (—Cu 2+) conditions, as was the untransformed control (Ctrl) strain. E. coli-produced proteins for Cry4A 700 (E. coli-4A) and Cry11A (E. coli-11A) were included as markers, and these versions also have a His-tag. For the Chlamydomonas samples, 75 μg of total cell protein was loaded in each lane. The gel was 10% acrylamide, and the locations of protein size markers are indicated. Flag antibody was used to probe the blot, and chemiluminescence was captured with X-ray film. (B) A Cry4B transformant was grown under Uninduced and Induced conditions and analyzed as in (A), except the gel was 6% acrylamide. Protein size markers are indicated.

FIG. 10 shows exemplary results for Western blot analysis of Cry transformants with the anti-Flag antibody. (A) Solubilized cells (20-m chlorophyll) were separated on a 12% polyacrylamide gel, blotted and probed with the monoclonal anti-Flag antibody. The Chlamydomonas strains were: Ind41_18, parental; 4A, Cry4Aa₇₀₀ transformant 4A-2; 4B, Cry4Ba-1 transformant 4B-1; 11A, Cry11Aa transformant 11A-8. Each strain was grown under uninduced and induced conditions for ˜72 hours. The non-specific band (NS) migrating at ˜145 kDa in all the lanes serves as a loading control. (B) Solubilized cells (10 μg chlorophyll) from the 4B-1 transformant, grown as indicated, were separated on a 6% polyacrylamide gel. Duplicate lanes were either stained with Coomassie (bottom panel) to verify the loading, or blotted and probed with the anti-Flag antibody (top panel).

FIG. 11 shows exemplary results from RT-PCR analysis of the Cry4Aa₇₀₀-2 (4A) and Cry11Aa-8 (11A) transformants. An equal amount of RNA from cultures grown for 72 hours under uninduced (U) and induced (I) conditions was used for reverse transcription with gene-specific primers; 796 for Cry4A₇₀₀ and 799 for Cry11A. The resulting cDNAs were amplified using primers 795+796 for Cry4Aa₇₀₀ and 799+800 for Cry11Aa. Reactions without reverse transcriptase in the RT step served as negative controls (lanes 2, 4, 7, 9). Also, PCR reactions with total nucleic acids (TNA) from both strains served as positive controls for the PCR step (lanes 5 and 10). Lane M contained size markers, and the gel image was inverted. RT, reverse transcriptase.

FIG. 12 shows exemplary effects of inducing Cry4Aa₇₀₀, Cry4Ba, and Cry11Aa on the growth rate of the transformants. The Ind41_18 parental strain (A) and the Cry4Aa₇₀₀-2 (B), Cry4Ba-1 (C) and Cry11Aa-8 (D) transformants were grown under uninduced (TAP+Cu²⁺) and induced (TAP−Cu2+) conditions. Growth was estimated by measuring total chlorophyll and converting to numbers of cells.

FIG. 13 shows exemplary live vs. dead mosquito larvae fed C. reinhardtii expressing inducible Cry11Aa from a novel gene of the present inventions. (A) A typical healthy A. aegypti larva fed Ind41_18. (B) Dead A. aegypti larvae fed Cry11Aa-8 grown under inducing conditions. The images were captured 4 days after feeding.

FIG. 14 shows exemplary lethality of the Cry4Aa₇₀₀ and Cry11Aa transformants to Aedes aegypti and Culex quinquefasciatus larvae. The Cry4Aa₇₀₀ (4A-2) and Cry11Aa (11A-8) transformants were grown under uninduced (U) and induced (I) conditions, whereas the control parental strain (Ind41_18) was grown under induced conditions. The assays were performed in dH₂O to prevent the algae from growing, and a dH₂O (Water) control (no algae) was included. The assays were performed in triplicate, each contained 10 larvae, either A. aegypti (A) or C. quinquefasciatus (B). Larval mortality was checked every 24 hours; the data are from 48 hours. 1×=1×10⁶ cells/mL.

FIG. 15 shows an exemplary diagram of pCry4A₇₀₀, pCry4B and pCry11A constructs and the site of integration in the chloroplast genome of wild-type C. reinhardtii. Each of the Cry genes have a Flag tag at the C-terminus, and are flanked by psbaₘ and psbA control regions. The locations of primers used for PCR are indicated; note that 864 and 865 are located upstream and downstream, respectively, of the integration site in CpDNA. Some parts of the diagram are not drawn to scale.

FIG. 16 shows exemplary PCR analysis of chloroplast transformants in a wild-type host. Analysis of three independent transformants that were co-transformed with either pCry11A (A) or pCry4B (B) and selected on spectinomycin. Total DNA was used for PCR with primers that either flanked the integration site (864/865), or were internal and gene-specific (799/800 for Cry11Aa, and 797/798 for Cry4Ba). Reactions with wild-type DNA were included to evaluate homoplasmicity at the integration site (864/865). Lane M contained DNA size markers.

FIG. 17 shows exemplary Western blots of the Cry11Aa wild-type transformants. The three Cry11Aa transformants from FIG. 8 (11Awt-7, 11Awt-8, 11Awt-11), and the untransformed host strain (Wild type) were grown in TAP medium in the light. Also, the inducible Cry11Aa transformant 11A-8 was grown under induction conditions (lane 6). Equal total cell fractions (4 μg chlorophyll, ˜60 μg protein) were loaded on the 10% gel, blotted and probed with the Flag antibody. E. coli expressing a His-tagged Cry11Aa (E. coli-11A) was included in lane 1 as a positive control. The positions of size markers are indicated to the left. The NS (Non-specific) band lights up with wild type cells (lanes 2-5), and not with Ind41-18, which is the host strain used for inducible expression (lane 6).

FIG. 18 shows exemplary growth curves of the Cry11Awt-8 transformant and host strain (Wild type). Cells were diluted to 5×10⁴ cells/mL in TAP medium and incubated in the light with shaking. The number of cells was counted every 12 h. Plotted are the averages±SEM from three independent trials.

FIG. 19 shows exemplary representative live (left) and dead (right) A. aegypti larvae. fed wild-type C. reinhardtii expressing Cry11Aa from a novel gene of the present inventions. (A) Typical healthy A. aegypti larva fed wild-type alga. (B) Dead A. aegypti larvae fed Cry11Awt-8 cells.

FIG. 20 shows an exemplary larval bioassay of a Cry11A wild-type transformant (Cry11A wt-8) with A. aegypti Ten 4^{th} instar larvae with live algal cells in dH2O) were used in each assay, which was in triplicate (n=3). Larval mortality was checked visually after 24 and 48 hours; the data are from 48 hrs of incubation.

FIG. 21 shows an exemplary Cry11Aa: Construct and modified synthetic gene used for transfecting both the inducible Ind41_18 strain and the wild-type Chlamydomonas strain.

FIG. 22 shows an exemplary Cry4Aa: Construct and modified synthetic gene used for transfecting both the inducible Ind41_18 strain and the wild-type Chlamydomonas strain.

FIG. 23 shows an exemplary Cry4Ba: Construct and modified synthetic gene used for transfecting both the inducible Ind41_18 strain and the wild-type Chlamydomonas strain.

FIG. 24 shows an exemplary schematic of a synthetic Cyt1Aa gene. A codon-optimized Cyt1Aa gene was synthesized using the DNA shuffling method with a FLAG tag at 3′ end. After optimization, the CAI reached 1.

FIG. 25 shows an exemplary in vitro-synthesized Cyt1Aa gene (774 bp) analyzed on a 1% agarose gel next to a lane of DNA size markers (lane M).

FIG. 26 shows an exemplary diagram of the Cyt1A gene construct and the site of integration in the chloroplast genome of strain CC-1690.

FIG. 27 shows exemplary PCR analysis of three Cyt1A transformants. The plasmid DNA (pCyt1A) and wild-type chloroplast DNA (CC1690) were used in parallel PCR reactions as specific markers; pCyt1A is the plasmid that was shot into the chloroplast. The other lanes contained DNA size markers (M), a reaction with no DNA (−) (as a negative control), and DNA from the three Cyt1A transformants. (A) Diagram of the PCR primers and expected sizes of the respective PCR products. (B) PCR products used to confirm the presence of the Cyt1A gene. (C) PCR products used to confirm homoplasmicity of the transformed chloroplast genome (i.e., the absence of the CC1690 band).

FIG. 28 shows exemplary Western blot analysis of a Cyt1Aa transformant (Cyt1A-1) with the anti-FLAG antibody. Total cellular protein (either 10, 20 or 40 micrograms) was separated on a 10% polyacrylamide gel, blotted and probed with the monoclonal antibody (conjugated to alkaline phosphatase). The locations and sizes of protein size markers are indicated to the left of the blot. Lanes 1 and 2 contained total protein from the Cyt1A transformant (Cyt1A-1), and lane 3 contained total protein from the parental wild-type strain CC1690.

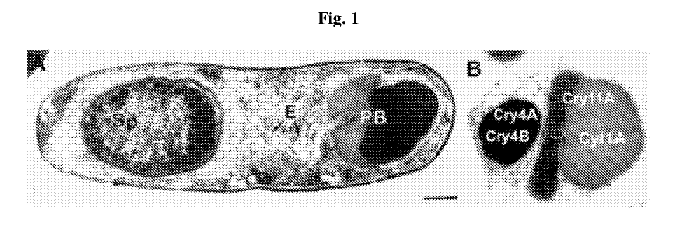

### DESCRIPTION OF THE INVENTION

The present invention relates to producing novel strains of green algae specifically engineered to produce an improved engineered compound over naturally occurring larvicide compound. In particular, genes were isolated and sequenced encoding naturally occurring larvicides produced by Bti (Bacillus thuringiensis subsp. israelensis), e.g. Cry proteins, were redesigned, synthesized, then introduced as heterologous transgenes into strains of Chlamydomonas reinhardtii for producing motile larvicidal-green algae specifically lethal to larvae of mosquitoes and black flies in water systems. Thus green algae (i.e. eukaryote) as motile biocontrol agents are contemplated for use to reduce the number of adult mosquitoes that transmit disease, such as West Nile virus, dengue, encephalitis, and malaria, in addition to reducing the number of adult black flies, in a safe and sustainable manner.

As described herein, effective, eco-friendly control of mosquitoes is contemplated by turning a preferred food source for the larvae, i.e. an edible eukaryotic green alga (Chlamydomonas), into a new biological larvicide by expressing protoxin genes from Bacillus thuringiensis subsp. israelensis (Bti) within the chloroplast. In particular, the use of Cry genes, individually and in mixtures (including but not limited to, for example, Cry4A+Cry4B and Cry4A+Cry11A) and further in combination with a Cyt1A gene, including but not limited to, for example, Cyt1A+Cry4A, Cyt1A+Cry11A and Cyt1A+Cry4A+Cry11A, are contemplated to provide strong toxicity to a wide variety of larvae of insects involved with causing disease in mammals. Combinations with Cyt1A are preferred as Cyt1A was shown to prevent the development of strong resistance to the lethal effects of Cyr toxins (10). Nonetheless, the inventors and others had previously failed at attempts to express protoxins from copies of native Cry bacteria genes, such as a Cry11A gene from Bti in the chloroplast of Chlamydomonas sp. as described below. Despite these failed attempts, the inventors' were subsequently able to express larvicidal levels of Cry protoxins in living algae, i.e. a modified Chlamydomonas laboratory strain and a wild-type Chlamydomonas strain cultured in the laboratory.

### I. Failure to Express Cry Proteins as Protoxins in Chloroplasts of Green Algae.

As described herein, initial attempts at expressing Bti Cry proteins in green alga failed to produce viable larvicidal-C. reinhardtii. In particular, the inventors initially used an atpX expression system to express an exemplary copy of a native Cry11A gene from Bti in the chloroplast. However, these transformed algae were not an effective larvicide due to failed protein expression.

Moreover, others were also unsuccessful at expressing protoxins from copies of Bti genes, see, Juntadech, et al., “Efficient transcription of the larvicidal cry4Ba gene from Bacillus thuringiensis in transgenic chloroplasts of the green algal Chlamydomonas reinhardtii.” Advances in Bioscience and Biotechnology, 3(4): 8 pages (Published Online August 2012). More specifically, in this 2012 publication successful transcription of a 3.4-kb mosquito-larvicidal cry4Ba gene, copied from a Bacillus thuringiensis gene, was expressed as a transgene in transformed C. reinhardtii chloroplasts under control of the promoter of the photosynthetic gene psbA and 5′-UTR/3′-UTR of psbA. However, the paper then reports that production of the protein was NOT accomplished, i.e. immunoblotting with the specific Cry4Ba-domain III monoclonal antibody revealed no demonstrable accumulation of the recombinant protein. Thus, because no protoxin was produced these transgenic C. reinhardtii strains were not larvicidal. Nonetheless, the Juntadech et al. paper proposed a solution to the problem: “It is therefore possible that the deficient translation of the high-yield cry4Ba transcript in transgenic chloroplasts could perhaps be due to biases seen in glycine and histidine codons used in this recombinant protein-coding gene. Hence, further studies via codon optimization of this non-native gene are of great interest since a codon-optimized cry4Ba gene might be indeed a requirement for improving the heterologous production of the Cry4Ba insecticidal protein in C. reinhardtii chloroplasts . . . .” Juntadech et al. also compared the codon usage of a Bti-Cry4Ba encoding gene to codon usage of genes in the C. reinhardtii chloroplast genome using world wide web//.kazusa.org then stated that “[p]atterns of synonymous codon usage in both the bacterial cry4Ba transgene and the C. reinhardtii chloroplast genome are quite similar as almost all codons ending in A [DNA] or U [RNA] are preferred.” emphasis added. Thus Juntadech was not helpful in suggesting successful ways to increase protoxins expression.

In contrast to the statements in Juntadech, et al., successful expression of a Cry protein was not as simple as Juntadech et al. proposed. In fact, some protoxins expression was found when the third codon in a codon modified novel Cry4Ba sequence of the present inventions has CAA instead of AAC in the native sequence, which changed the translated aa to glutamine (Q) from asparagine (N) in the expressed protein.

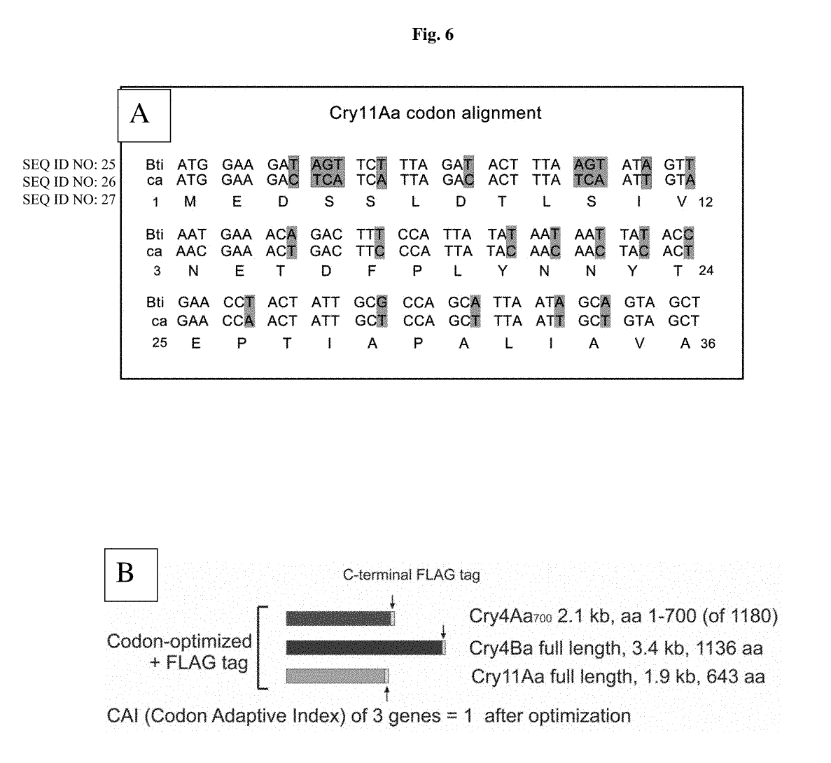

In fact for Cry11Aa, Glycine and Histidine codons were not present in the first 36 amino acids of the novel Cry11Aa gene of the present inventions, instead the inventors' modified codons as described below, for several other amino acids, such as at A, D, S, I, P, and V, including increasing the use of C (DNA)/G (RNA) in the wobble positions at the end of the codon. See shaded areas in an exemplary novel Cry11Aa (ca) compared to an isolated copy of a Bti Cry11Aa sequence (FIG. 6A).

Further, the inventors ligated 5′ and 3′ expression signals (from psbD and psbA, respectively) to their novel Cry genes. An additional modification was made to the native psbD sequence, a possible Shine-Dalgarno sequence in the 5′ UTR, GGAG, was modified to AAAG (creating 5′ psbDm) to decrease translation in E. coli without expecting an effect on chloroplast translation. For tagging expressed proteins, a FLAG tag sequence was ligated to the novel Cry synthetic genes of the present inventions, see for examples, FIG. 6B. Additionally, novel sequences of the present inventions, including but not limited to proteins related to Bti Cry11A, Cry4B, and Cry4Aa were then entirely synthesized as DNA templates for copying and using in the transgenic organisms of the present inventions.

The inventors' initially attempted to express these synthetic and novel Cry genes within the chloroplast by adapting the approach of the S. Mayfield lab, see Example I. However, merely a fraction of the native chloroplast DNA molecules encoding CRY were expressed in the transformants with the engineered DNA copies, even after many rounds of selection.

More specifically, the inventors were not able to produce Cry4A protein (Cry4A-700) from their first attempt at transforming a wild-type strain using synthesized genes having codon modifications when compared to isolated Bti gene sequences. This result indicated in part that the constitutive high-level expression of Cry proteins afforded by this system was too toxic to the organism. Therefore, codon optimization was not enough for the successful production of Cry proteins within chloroplasts.

In contrast, by using cyanobacterium (Anabaena) as a host for larvicidal genes, the inventors (and others) were able to engineer it to express at least two Cry genes and Cyt1A (refernces 9, 47), which made this transgenic bacterium highly lethal to mosquito larvae. See, S. Boussiba et al., “Nitrogen-fixing cyanobacteria as gene delivery systems for expressing mosquitocidal toxins of Bacillus thuringiensis ssp. israelensis.” J. Appl. Phycol. 12:46-467 (2000).

However, the use of cyanobacterium as larvicides has several limitations, including but not limited to the fact that they are prokaryotes lacking organelles which in part allows the escape of transgenes into the environment. Further, prokaryotes frequently pass on or exchange DNA sequences also allowing transgenes to spread horizontally. Thus eukaryotes have an advantage that organellar location of the transgenes provides better gene containment than in a cyanobacterium. Further, a cyanobacterium organism, which was produced in Israel and patented, has not been deployed in the field (49). Apparently, a principal reason for this concerns the bacterial antibiotic-resistance genes that were used to obtain the cyanobacterial transformants (47,49); as organisms containing heterologous bacteria transgenes are now strongly discouraged for use in transgenic organisms released into the environment. Moreover, communities in Europe and Africa are resistant to the release of transgenic organisms. Indeed, the United States is one of the few countries that have allowed the deployment of transgenic bacteria in the environment (in particular for bioremediation).

In light of the concern of releasing transgenic organisms into the environment that contain a variety of bacteria regulatory genes and/or anti-biotic resistant genes, another advantage of this system is the lack of, or reduction of, the use of bacteria regulatory genes in the larvicidal-Chlamydomonas of the present inventions. In fact, the Chlamydomonas reinhardtii in Juntadech, et al., supra, expressed an actual cry4Ba bacteria gene in addition to a gene providing resistance to spectinomycin ((aadA, encoding aminoglycoside adenyl-transferase which confers resistance to spectinomycin treatment)).

To evaluate the potential for chloroplast-based expression of the protoxins, an inducible Cyc6-Nac2-psbD expression system and synthetic codon-optimized Cry genes was used. Also, the Cry4A gene was truncated after amino acid 700, creating Cry4A 700, and all 3 proteins were Flag-tagged at the C-terminus. The genes were outfitted with the psbD 5′ control region and integrated into the chloroplast genome of the Ind41_18 strain; homoplasmic transformants for each gene were confirmed by PCR.

Analysis with western blots of whole cells showed that all 3 Cry proteins could accumulate and were increased by induction (i.e., —Cu 2+) conditions; the induced expression levels, in order, were Cry4A 700>Cry11A>Cry4B. The induced Cry4A 700 and Cry11A strains were toxic to Culex sp. and Aedes aegypti larvae in a live cell bioassay, with the more-toxic Cry11A strain giving an LC 50 of 3.3×10 5 cells/mL with A. aegypti larvae.

### II. Cry Gene Compositions and Methods for Successful Production of Larvicidal-Chlamydomonas.

As described herein, synthetic genes for the Cry4Aa, Cry4Ba, Cry11Aa and Cyt1A encoding proteins were made as synthetic DNA transgenes derived from looking at the amino acid sequences of Bti Cry proteins during the development of the present inventions. These specific Cry toxins were chosen because each has the capability to kill larvae, however they have different effectiveness and different toxicity to different species of mosquitoes. In other words, each type of Cry protein has a different level of toxicity towards each species of mosquitoes. As an example, Cry11A is the most effective (i.e. requires the least amount to kill) when used alone showing lethal activity against the majority of mosquito species. The inventors discovered that when the Cry proteins are mixed together they show synergy of action (i.e., increased larvicidal activity with the mix rather than merely an additive level of activity of the individual components). However, adding Cyt1A resulted in a higher lethality than a mixture of Cry4Aa, Cry4Ba, Cry11Aa without Cyt1A. Therefore in a preferred embodiment, larvicidal-algae of the present inventions comprise a heterologous gene expressing Cyt1A protoxins.

As described herein, Bt native genes were redesigned then synthesized. In one embodiment, novel genes for encoding Cry larvicides are used as heterologous transgenes for expressing larvicides in green alga. Additionally, novel modifications are contemplated for adding i.e. ligating into the coding sequences or for co-expressing, such as different promoter, regulatory sequences, etc. Further, the use of engineered Cry proteins, such as a Cry19A derivative that has broad toxicity (1), or other activators (8,43,46) may be utilized by co-expression systems or from ligating to the Cry coding sequence for duel expression. The following provides more specific information on the genes and proteins made and used during the development of the present inventions.

A. Designing and Synthesizing DNA Sequences for Use as Transgenes in Chlamydomonas.

As described herein, the codon usage of the novel synthetic gene of the present inventions was changed from the codon usage of isolated sequences of Bti Cry genes using methods not described by Juntadech et al. Instead, the inventors used reverse engineering for designing the encoding DNA in addition to using the information found from analyzing organelle codon usage from 8 highly expressed genes in the Chlamydomonas chloroplast DNA. Thus, in one embodiment, novel gene sequences encoding Cry4Aa, Cry4Ba, and Cry11Aa proteins based upon chloroplast organelle codon usage are provided herein for use in making larvicidal-Chlamydomonas. Further, as described herein, additional modifications to the methods of producing larvicidal-Chlamydomonas, including using a different expression system, using different regulatory sequences, using truncated fragments of CRY or CRT proteins, etc. were necessary to use for at least some of these proteins to have expression levels at effective levels in viable host organisms.

1. Cry Toxins and Cyt Proteins.

B. thuringiensis bacteria comprises at least 19 varieties with numeorus supspecies having numerous genes capable of encoding Cry toxins and Cyt proteins. Cry toxins from each variety or subspecies are slightly different as they have different levels of toxicity to a range of different organisums. Several of these toxins were used in the production of biological insecticides and their genes in insect-resistant genetically modified crops. When insects ingest toxin crystals, their alkaline digestive tracts denature the insoluble crystals, making them soluble and thus amenable to being cut with proteases found in the insect gut, which liberate the toxin from the crystal. The Cry toxin is then inserted into the insect gut cell membrane, paralyzing the digestive tract and forming a pore. The insect typically reduces eating and starves to death; live Bt bacteria may also colonize the insect which can also contributes to death when digested to release the toxins. Insecticidal activity of the various Cry genes includes but is not limited to toxic effects upon dipterans (flies and mosquitoes), lepidopterans (butterflies and moths), coleopterans (beetles), hymenopterans (wasps and bees), nematodes, etc. Examples of Cry Bt toxin genes and proteins contemplated for use in developing larvicides of the present inventions, includes but are not limited to Cry1Aa, Cry1Ac, Cry2Aa, Cry3Aa, Cry3Ba, Cry4Aa, Cry4Ba, Cry11Aa, etc. Thus in one embodiment, a host larvicidal-green alaga would have a higher concentration of a larvicide more specific for mosquitoes or black flies or both. Therefore, in one embodiment, a host larvicidal-green alaga expresses Cry toxins as larvicides specifically for targetng larvae of mosquitoes and black flies.

Unlike each Cry toxin which has specific actions against certain order of insects, such as Lepidoptera and Coleoptera vs. dipterans (flies and mosquitoes), etc., Cyt proteins are toxic in vivo to the larvae of members of the order Diptera, such as mosquitoes and black flies. In vitro it exhibits broad cytolytic activity against a variety of insect and mammalian cells, including erythrocytes, lymphocytes, and fibroblasts. It is contemplated that Cyt protein toxins act via formation of transmembrane ionic channels and/or pores which may explain why targets do not develop resistance, unlike the receptor-mediated action of Cry toxins.

2. Cry and Cyt Genes of the Present Inventions.

The inventors further contemplate producing larvicidal-Chlamydomonas strains expressing at least 2 of 3 Cry toxin proteins (for example, Cry4Aa, Cry4Ba, and Cry11A). These Cry proteins show a high level of toxicity to mosquito larvae. Thus, in some embodiments, Chlamydomonas express a Cry toxin selected from Cry4Aa, Cry4Ba, Cry11A toxin protein. However, the addition of a Cyt1Aa protein increases their activity synergistically in other organisums, and prevents the development of highly-resistant mosquitoes to these Cry toxins. Thus in some preferable embodiments, larvicidal-Chlamydomonas strains of the present inventions additionally express a codon-adapted Cyt1Aa toxin protein.

a. Codon Modification of a Bti Gene for Optimizing Expression in C. reinhardtii Chloroplasts.

Within a genetic code, many amino acids are encoded by more than one codon, with the differences in specific codon usage varying from species to species as codon bias. The codon bias of an organism or particular genome is usually related to an organism's tRNA pool (Gustafsson et al., 2004). Thus, chloroplast of C. reinhardtii prefers adenine (A) or uracil (U) nucleotides in the wobble position, thus contributing to a high A-T content of the genome (Franklin et al., 2002; Rosales-Mendoza, 2011). A codon usage database for chloroplast-encoded ORFs is available online (Nakamura et al., 2000). However, the inventors' created a new codon substitution table for use in codon modification of the present inventions based upon the codons used by 8 highly expressed chloroplast genes.

The codon adaptation index (CAI) is a measure of codon usage bias, and can be used to predict whether heterologous genes will be expressed (Sharp and Li, 1987; Surzycki, 2009). CAI values vary from 0 to 1, where 1 indicates that all codons in a gene are the most frequently used (Stenico et al., 1994).

Codon optimization is a process that changes codons of a transgene into the most commonly used codons in a host organism (Gustafsson et al., 2004), so that the CAI value increases close to 1. In this project, sequences of native Cry4Aa, Cry4Ba, and Cry11Aa genes were converted into codon-optimized sequences with Optimizer, a computer application developed by Puigbò et al. (2007). Expression of codon-optimized transgenes can increase protein levels dramatically, and has been successful in various hosts, including bacteria, plants, and mammals (Gustafsson et al., 2004), and in the C. reinhardtii chloroplast. Franklin et al. (2002) claimed an 80-fold increase in GFP accumulation by re-synthesizing the gfp gene to agree with the codon bias of C. reinhardtii chloroplast genes. Codon-optimized luciferase reporter genes from Vibrio harveyi and firefly resulted also in high expression of the reporter gene (Mayfield and Schultz, 2004; Matsuo et al., 2006).

Thus, for each gene, a synthetic gene encoding for a Cry toxin was made to have a codon-usage pattern closer to the codons used in chloroplast protein genes of Chlamydomonas. In fact, the Codon Adaptation Index (CAI) of the Cry genes synthesized during the development of the present inventions, increased from −0.5 to 1 after codon-usage optimization. Thus, a Bti protein sequence for each Cry gene was used to reverse engineer a novel Cry encoding gene. Therefore, in one embodiment, a novel Cry gene, such as Cry11, was made derived from a Bti Cry11 protein sequence.

b. Effect of the 5′ and 3′ Untranslated Regions (UTR) on Expression.

The native chloroplast genes are regulated at transcriptional, post-transcriptional (RNA stability, processing, and splicing) and translational levels (Rochaix, 1996). Besides the transcriptional promoters, the 5′ and 3′ UTRs that flank the transgene are determinants of expression; the 5′ UTR affects translation and sometimes mRNA stability, while the 3′ UTR mostly affects mRNA stability (Herrin and Nickelsen, 2004). Translational factors and ribosomes interact with the 5′ UTR in mediating translation of an mRNA (Rochaix, 1996; Harris et al., 1994). The 5′ UTR and the 3′ UTR form stem-loop structures that bind proteins protects the transcripts from exonucleases and determine the 3′ end of the mRNA (Herrin and Nickelson, 2004).

There have been several studies on the relationship between transgene expression and the specific 5′ and/or 3′ UTR that is on the reporter gene (Ishikura et al., 1999; Barnes et al. 2005; Michelet et al., 2011; Rasala et al., 2011). For examples, Barnes et al. (2005) found that the 5′ UTRs from the atpA and psbD genes gave higher levels of GFP than the 5′ UTRs from the rbcL and psbA genes, but that various 3′-UTRs hardly affected GFP protein accumulation. Probably, the highest level of any foreign protein was obtained when the 5′ and 3′ expression signals on the transgene were from the psbA gene, and the transgene replaced the endogenous psbA gene (instead of an ectopic insertion) (Minai et al., 2006). Apparently, an autofeedback mechanism involving the psbA protein normally restricts translation (Minai et al., 2006; Manuell et al., 2007). The disadvantage of this approach, however, is the loss of photosynthesis caused by replacing native psbA with the transgene. To restore photosynthesis, the psbA gene with a non-native 5′ UTR has to be inserted in another location. The lesson from these studies is that competition between endogenous genes and transgenes for limiting factors may limit protein expression levels.

c. Other Factors that can Affect Expression.

Light can regulate the translation of chloroplast transgenes that have a 5′ UTR from photosynthesis genes. Synthesis of GFP (Green Fluorescent Protein) driven by 5′ UTRs from psbA or psbD was increased under high light flux compared to cultures kept in darkness (Barnes et al., 2005; Rasala et al., 2010).

Other aspects of the coding region besides codon usage can affect translation efficiency (Herrin and Nickelsen, 2004), and of course, there is protein stability. Different foreign genes flanked by the same 5′/3′ UTRs can vary greatly in the level of recombinant protein accumulation (Surzycki et al., 2009). An up to 3-fold higher level of bacterial β-glucuronidase (GUS) was achieved when the beginning of a native chloroplast gene was fused to the N-terminus of GUS (Kasai et al., 2003). Barnes et al. (2005) suggested that RNA-RNA interactions between the coding region and the 5′ UTR might affect the local secondary structure and binding of translation factors. In at least one case, fusing a small protein to the C-terminus of the coding sequence enabled the accumulation of an apparently unstable recombinant protein (Rasala et al., 2010).

Lastly, the genetic background of the host strain can affect the level of transgene protein, at least for nuclear genes and probably for chloroplast genes (Fletcher et al., 2007). Two transformed host strains (137c and cc744) of C. reinhardtii exhibited different levels of luciferase accumulation with the same chloroplast transgene (Mayfield and Schultz, 2004).

B. Methods of Transfecting Transgenes and Developing Larvicidal Strains of Chlamydomonas.

After designing and synthesizing novel Cry toxin encoding genes, including Cyt1A genes, these genes were transformed into the chloroplast genome for producing a Chlamydomonas strain that has the Cry transgene gene under inducible control (44). Insertion is contemplated to occur through homologous recombination. This inducible expression system allows the verification of the synthetic gene's function, while gauging the potential for wild-type expression and possible toxicity to the host cell. In a wild-type strain, expression of the protoxin would not require induction, though it would be influenced by the light-dark cycle. When the results from expressing a particular Cry gene sequence using the induction system were sufficiently encouraging, then duplicate copies of that synthetic Cry gene were then transfectede into the chloroplast of a wild-type strain. Its homoplasmicity, expression level and stability were then verified.

1. Inducible Expression of Cry11Aa and Cyt1Aa-p20 in the Chloroplast.

Expressing Cry11Aa and Cyt1Aa (and p20):

In addition to Cry4Aa and Cry4Ba, the toxicity of Bti bacteria includes toxins Cry11Aa (72 kD) and Cyt1Aa (27 kD). Whereas Cry11Aa shares similarities with Cry4Aa and Cry4Ba (18), Cyt1Aa differs from the Cry proteins in that it does not bind a specific gut receptor, but acts non-specifically (10,43). By itself, Cyt1Aa is not highly toxic, but it is strongly synergistic with the Cry proteins (21); synergy has also been noted for Cry4Aa+Cry4Ba, and Cry11Aa+, either Cry4Aa or Cry4Ba (1,33). Cyt1Aa prevents the development of mosquitoes that are highly resistance to Bti (39,43).

The p20 gene is located adjacent to Cry11Aa on the pBtoxis plasmid (21); it has been suggested that it is a chaperone, but that may be a misnomer. In any case, p20 binds to Cyt1Aa and blocks its lethal effects in E. coli. Moreover, it promotes the accumulation of Cyt1Aa and Cry11Aa in bacterial hosts (45). p20 is not required for Cyt1Aa accumulation in the chloroplast. Thus, the inventors contempalte expressing Cry11Aa and Cyt1Aa (with and without p20) in the chloroplast, and further contempalate adding the expression of Cry4Aa₇₀₀ and/or Cry4B.

Using the Inducible Chloroplast-Expression System:

The inducible NAC2/psbD system is contemplated for use in expressing Cry4Aa₇₀₀ and Cry4Bais for producing potentially toxic proteins from the chloroplast genome. With this system, chloroplast transformants were generated that do not express the psbD-driven target gene until the Cyc6::NAC2 nuclear gene is induced by depleting Cu²⁺ from the medium (41). Although the kinetics of this induction are relatively slow (24-48 hrs to maximum levels), it is efficacious, and allows determination of whether expression of a given protein inhibits cell growth. Also, if the target protein does not accumulate, the problematic step (transcription, mRNA stability, translation or protein stability) can be identified, and often remedied. The current technology avoids major problems with transcription, translation and mRNA instability by using codon-adapted genes and the proper 5′ and 3′ expression signals (11,36). However, for Cyc6::NAC2 control, the 5′ untranslated region (5′-UTR) of the target gene must be from the psbD gene; chloroplast mRNAs with this 5′-UTR are highly unstable unless bound by NAC2 (17). Finally, the Cyc6 promoter is repressed by the Cu²⁺ in the standard medium; so, once the culture has grown to the desired density, Cyc6::NAC2 is de-repressed (induced) by changing the medium from +Cu²⁺ to −Cu²⁺ (41).

a. Inducible Expression of Cry11Aa.

A Cry11Aa gene whose codon-usage closely matches the codon-usage of the Chlamydomonas chloroplast was designed and synthesized during the develpoment of the present inventios. This Cry11Aa protein (amino acid) sequence used for this design is native (as in found from Cry11Aa proteins made by Bti encoding genes), except for a small tag (Flag) on the C-terminus, which is a marker used for detecting and quantifying Cry11Aa proteins with the anti-Flag antibody.

For inducible expression Cry11Aa is contempalted for construction as with the Cry4 genes, with the psbD promoter/5′-UTR at the 5′ end, and the psbA 3′-UTR at the 3′ end. For chloroplast integration, the psbD::Cry11Aa::psbA gene is cloned into a chloroplast transformation plasmid similar to the one used for the Cry4 genes by the inventors. Briefly, the gene is imbedded in a 5-kb fragment of chloroplast DNA (in p322), which will recombine (in its flanks) with the homologous region of the genome and replace it (3). Transformants will be selected by using co-transformation with a chloroplast 16S rRNA gene that confers spectinomycin resistance (14), or by direct selection of an aadA marker that is integrated next to the psbD::Cry11Aa::psbA gene (16).

b. Expression of Cyt1Aa.

Cyt1Aa is a Bacillus thuringiensis ssp. israelensis toxin protein that is contemplated to synergistically increase the lethality of the Cry proteins when expresed in Chlamydomonas reinhardtii chloroplasts. It has weak cytolytic activity against certain cell types, which depended on the phospholipids in their cell membrane (Federici et al., 2003). In other systens, toxicity of Cyt1Aa, without other toxins, against mosquito larvae is weak, compared to the Bti Cry proteins, Cry4Aa, Cry4Ba, and Cry11Aa. In contrast, Cyt1Aa suppresses the development of resistance in mosquito larvae exposed to Bti toxins. Toxicity of Cyt1Aa is mediated by a toxin-lipid interaction rather than by the toxin-receptor interaction that mediates the toxicity of Cry proteins (Butko, 2003). Moreover, Cyt1Aa can act as a receptor for Cry4Ba and Cry11Aa.

Hence, we contemplated expression of Cyt1Aa in the chloroplast of Chlamydomonas reinhardtii, in order to complement our success in expressing the Cry protein genes, and because it may be provide the best possible mosquito larval biocontrol organism.

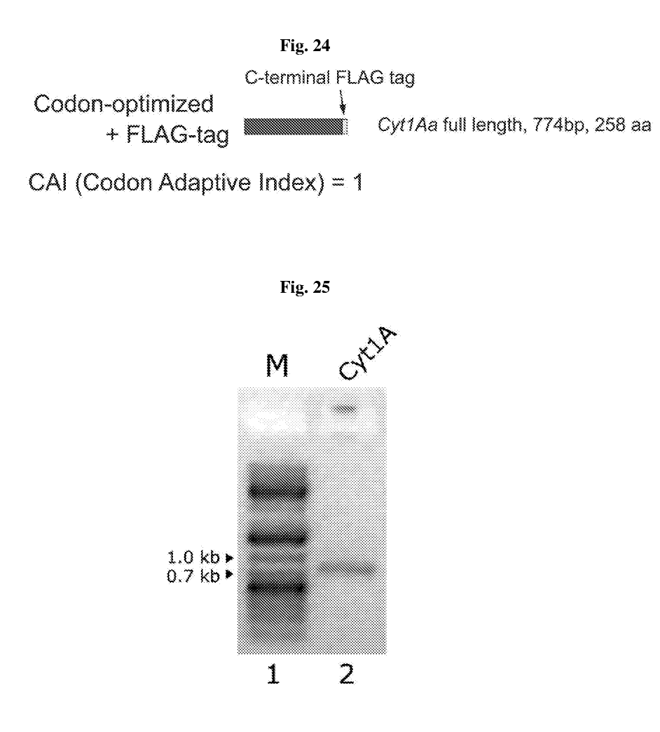

The Cyt1Aa DNA sequence (774 bp) of B. thuringiensis ssp. israelensis (NCBI NC_010076.1) was optimized using the program Optimizer (Puigbo, et al., 2007) and a codon-usage table of the chloroplast of Chlamydomonas reinhardtii (Nakamura, et al., 2000). A FLAG tag sequence was added to the 3′ end of the Cyt1Aa sequence (FIG. 24). A nucleotide at position of 296 was changed from adenine to thymine for the ease of cloning by removing NdeI restriction site. The codon-optimized coding sequence: DNA sequence of the codon-optimized Cyt1Aa gene with FLAG tag is SEQ ID NO:18.

Cyt1Aa Condon-Optimized with FLAG Tag: SEQ ID NO:18:

ATGGAAAATTTAAATCATTGTCCATTAGAAGATATTAAAGTTAATCCATG
GAAAACACCACAATCAACAGCTCGTGTTATTACATTACGTGTTGAAGATC
CAAATGAAATTAATAATTTATTATCAATTAATGAAATTGATAATCCAAAT
TATATTTTACAAGCTATTATGTTAGCTAATGCTTTTCAAAATGCTTTAGT
TCCAACATCAACAGATTTTGGTGATGCTTTACGTTTTTCAATGCCAAAAG
GTTTAGAAATTGCTAATACAATTACACCAATGGGTGCTGTTGTTTCTTAT
GTTGATCAAAATGTTACACAAACAAATAATCAAGTTTCAGTTATGATTAA
TAAAGTTTTAGAAGTTTTAAAAACAGTTTTAGGTGTTGCTTTATCAGGTT
CAGTTATTGATCAATTAACAGCTGCTGTTACAAATACATTTACAAATTTA
AATACACAAAAAAATGAAGCTTGGATTTTTTGGGGTAAAGAAACAGCTAA
TCAAACAAATTATACATATAATGTTTTATTTGCTATTCAAAATGCTCAAA
CAGGTGGTGTTATGTATTGTGTTCCAGTTGGTTTTGAAATTAAAGTTTCA
GCTGTTAAAGAACAAGTTTTATTTTTTACAATTCAAGATTCAGCTTCATA
TAATGTTAATATTCAATCATTAAAATTTGCTCAACCATTAGTTTCATCAT
CACAATATCCAATTGCTGATTTAACATCAGCTATTAATGGTACATTAGAC
TACAAAGACGACGACGACAAATAA.

The codon-optimized coding sequence SEQ ID NO:18 was used as the basis for designing primers for gene assembly, which were 50 nucleotides in length and contained 25-nucleotide overlaps with the flanking primers in the opposite orientation. Cyt1Aa was synthesized using those primers and DNA shuffling method (Stemmer, (1994) DNA shuffling by random fragmentation and reassembly: in vitro recombination for molecular evolution. Proceedings of the National Academy of Sciences of the United States of America 91, 10747-10751) (FIG. 25). The mixture of primers were elongated and amplified using the Phusion DNA polymerase (NEB). The first product was purified using GenElute™ PCR Clean-Up (Sigma-Aldrich) and used for the template DNA of the second PCR with outside primers to produce only full-length Cyt1Aa. The in vitro-synthesized Cyt1Aa was ligated into pBluescript for cloning using Nde I (on the 5′ side) and Xba I sites (on the 3′ side). For recloning, the Cyt1Aa was excised from pBluescript using Xba I, blunting with the Klenow DNA polymerase, and then digestion with Nde I. To produce pET-Cyt1Aa, the Cyt1Aa was ligated to the pET-16B vector that had been cut with Bam HI (on the 3′ side), blunted with the Klenow DNA polymerase, and digested with Nde I (on the 5′ side). The nucleotide sequence of pET-Cyt1Aa was confirmed by Sanger sequencing (University of Texas at Austin DNA Facility).

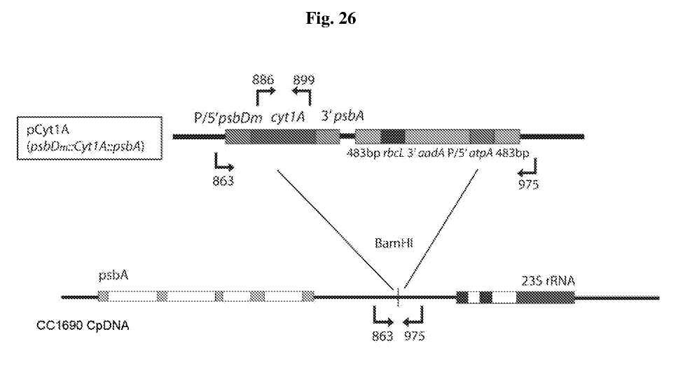

For chloroplast expression, the psbaₘ 5′ region and the psbA 3′ region used for the Cry genes were ligated to the 5′ and 3′ ends of Cyt1Aa in pET-Cyt1Aa. Then, the psbaₘ-Cyt1Aa-psbA gene construct, which had been excised with BamHI (on both sides), was cloned into the chloroplast expression vector, p322-483aadA, yielding plasmid pCyt1Aa (FIG. 26). The p322-483aadA vector had been generated by inserting the recyclable selectable marker for the Chlamydomonas chloroplast, 483aadA (Fischer, et al., 1996), into plasmid p322. The pCyt1Aa DNA was bombarded into the chloroplast of a wild-type strain of Chlamydomonas reinhardtii, CC1690, as described for the Cry gene expression.

Methods of protein extraction and analysis are briefly described. For the extraction of total cellular protein, 50 mL (or 30 mL) of transformed Chlamydomonas culture was pelleted by centrifugation at 2,000 rpm for 10 minutes (Heraus Centrifuge) at room temperature. The pellet was resuspended in 1 mL of lysis buffer (100 mM Tris-HCl pH 8.5, 100 mM DTT, 7 mM Benzamidine, and 5 mM EDTA pH 8.0). For the protein gel, 0.6 mL of cell lysate was treated with 0.4 mL of LDS buffer (5% lithium dodecylsulfate, 30% sucrose, and 0.025% Bromophenol blue). The preparation was stored at −70° C. (in 60 mM Tris-HCl pH 8.5, 60 mM DTT, 4.2 mM Benzamidine, 3 mM EDTA, 2% lithium dodecylsulfate, 12% sucrose, 0.01% bromophenol blue). Aliquots were loaded onto 10% polyacrylamide-SDS gels, and after separation, the proteins were electrotransferred to a PVDF membrane. The protein blots were probed with an anti-FLAG monoclonal antibody coupled to alkaline phosphatase, and detected with a chemiluminescent substrate and X-Ray film.

The total protein concentration of the cell lysates was determined with the Bradford reagent (Bio-Rad Protein Assay, Bio-Rad). To prepare the protein and remove the chlorophyll, the transformed Chlamydomonas culture cells were pelleted by centrifugation at 16,000 rpm for 5 min at room temperature, followed by resuspension in 90% acetone. The samples were then mixed, incubated for 2-3 min and centrifuged. The protein pellet was resuspended in Tris-HCl pH 8.0, 1% SDS and heated at 60° C. for 2-3 min. The samples were subject to the Bradford (Bio-Rad Protein Assay, Bio-Rad) using IgG for the standard curve.

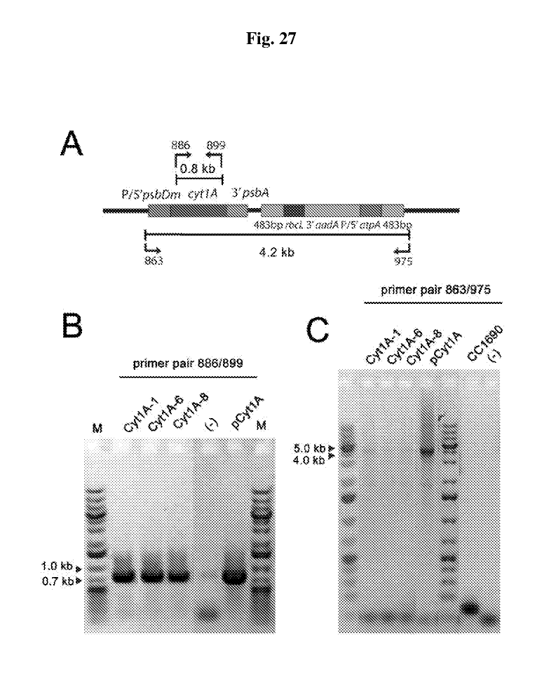

Chlamydomonas transformants are homoplasmic such that they have transformed copies of the chloroplast genome; i.e., there are no untransformed copies of CC1690 chloroplast DNA left as evidenced by the absence of the small PCR product that is indicative of CC1690 DNA (FIG. 27 panel C). FIG. 27 shows exemplary PCR analysis of DNA from three Cyt1A chloroplast transformants.

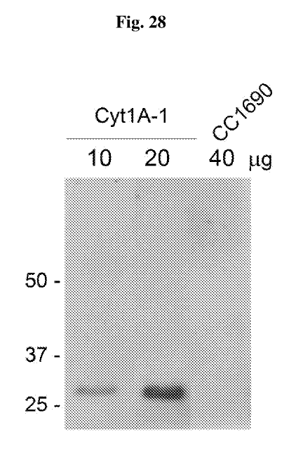

To visualize the Cyt1A protein in the algae, western blotting was used with a monoclonal antibody to the FLAG tag at the end of the protein. The western blot in FIG. 28 shows exemplary results of one of the three Cyt1A transformants. The blot shows a strong specific protein band of the estimated size for the Cyt1A protein in the transformant.

c. Inducible Expression of Cyt1Aa-p20.

Codon-optimized versions of Cyt1Aa (27 kD) and p20 (20 kD) were synthesized commercially as for the Cry4 genes.

In addtition to expression of Cyt1Aa, co-expression of Cyt1Aa with p20, is contemplated for use in the present inventions. In one embodiment, co-expression is inducible, i.e. one or both genes have inducible promoters. In another embodiment, co-expression is constituative.

p20 is contemplated to have an epitope tag (such as Flag or HA) at its C-terminus. Codons will be optimized using the program Optimizer with the codon usage of 8 strongly-expressed chloroplast genes of Chlamyomonas. During the development of the present inventions, it was foud that there is relatively little difference between the codon usage of these 8 genes and of all chloroplast genes together. As both of these proteins are relatively small, a fusion protein combining both proteins is contemplated in addition to expressing them as two separate genes on the same DNA fragment. The fusion protein approach would reduce the number of transgenes and number of necessary releated expression signals.

The two proteins in the Cyt1Aa-p20 fusion will be separated by a linker peptide that contains the cleavage site for a chloroplast endoprotease (30). This linker peptide was used to express a mammalian protein as a fusion to rbcL in the Chlamydomonas chloroplast; most of the fusion protein was properly cleaved (30). Inducible expression of Cyt1Aa-p20 in the chloroplast will be accomplished as described above for Cry11Aa, with a psbD::Cyt1Aa-p20::psbA construct in the chloroplast transformation plasmid p322, and the Ind41-18 strain, which has the inducible Cyc6::NAC2 gene (41).

Analysis of chloroplast transformants—Primary transformants are subjected to 3 rounds of colony selection and growth on selective plates before DNA analysis, which will be by PCR and/or Southern blot hybridization.

d. Identification of 5′ Expression Signals for Normalized Expression of Cry11Aa and Cyt1Aa (p20).

A psbD 5′-UTR is contemplated for use on new genes in the chloroplast without losing expression efficiency due to competition for trans-acting factors like NAC2. Additional chloroplast genes are contemplated for expression signals, in particular for the 5′-UTRs of the transgenes. Fortunately, in Chlamydomonas the expression signals of many chloroplast genes, including several that are highly expressed, have been used to express foreign genes (references 2, 3, 14, 27, 28, 36, 47).

An unsuccessful attempt was made using the very high-expressing system used by the Mayfield lab for producing human therapeutic proteins (35). With this system we got a low copy number for the introduced transgenes despite drug selection, suggesting that they were toxic. However, there are multiple unique features of that system that might be relevant, including: (1) extremely high expression levels, (2) localized translation on the thylakoid membrane (which might have facilitated membrane damage), and (3) a constant state of stress, because of the psbA coding region deletion (35). With our current approach, (3) is no longer relevant (since we do not delete anything and photosynthesis is maintained); (1) is less relevant, and by choosing the right 5′-UTRs, we should be able to reduce or eliminate (2). In that vein, the rbcL gene signals may be ideal; the mRNA is translated mainly around the pyrenoid (42), which is a substructure that contains the rbcL and rbcS subunits of ribulose-1,5-bisphosphate carboxylase, and the gene is strongly expressed (2). Thus, in one embodiment, the 5′ and 3′ signals from rbcL will be used for control regions.

There are other chloroplast genes whose 5′ expression signals are as effective as those of rbcL, such as atpA (36). And there are 5′ control regions that give a somewhat lower level of expression, but are nonetheless robust, such as those from petA or tufA (47). The promoter/5′-UTR regions from this latter group of genes may be preferred if the Cry11Aa or Cyt1Aa protoxins show evidence of host-toxicity with the inducible expression system.

2. Selection and Analysis of Chloroplast Transformants.

Clones that appear to be homoplasmic (i.e. copies of the genome are the same) will be used in the induction assay, which will include protein analysis (western blotting with specific antibodies), mRNA analysis, and bioassays with mosquito larvae. Cry11Aa and p20 are contempalted to have an epitope tag. However Cyt1Aa may not. Instead, to detect free Cyt1Aa a polyclonal antibody elicited with purified, His-tagged protein from E. coli will be used. Since the Cyt1Aa antibody should not cross-react with the Cry proteins (39), it will be used for quantifying Cyt1Aa in transformants expressing Cry genes. The Cry11Aa and Cyt1Aa proteins purified from E. coli will be used as reference standards for the quantitative western blots.

Partitioning of the proteins into soluble versus insoluble fractions is contemplated, using standard techniques for cell homogenization and differential centrifugation, but taking care to distinguish between insoluble and membrane-associated proteins.

Bioassays will be performed with live algae (and other materials as needed) and mosquito larvae reared in our lab (40); at least 2 genera, Culex and Anopheles (gambiae), will be grown for the bioassays, with more species contemplated for testing with Chlamydomonas strains that express protoxins. Sporulating Bti is contemplated as one bioassay standard (13) however transgenic E. coli with published toxicity values (21) may also be used. For each algal strain, LC50 and LC90 values, as well as time-course data will be obtained using methods as described by others (13,38,44).

3. Normalized Expression of Cry and Cyt Genes in Wild-Type Strain Alga.

A wild-type strain of C. reinhardtii, 2137 (CC-1021 wild type mt+), was obtained from the Chlamydomonas Center (U. of Minnesota). Strains were grown in TAP medium in the light (40 μE m⁻² sec⁻¹) at 23° C. with shaking. Cell number for the wild-type transformants was estimated from total chlorophyll using the reference value of 4 mg chlorophyll per 1×10⁹ cells (Harris, 1989).

a. Normalized Expression of Cry4Aa₇₀₀ and Cry4Ba₆₇₅ with the psbD Promoter/5′-UTR.

We demonstrated chloroplast-based expression of Cry4Aa₇₀₀ and Cry4B with the inducible system, and did not see any evidence of growth inhibition even under prolonged induction conditions. Thus, the toxicity of those protoxins to the host algal cell, at least when expressed with the psbD 5′ signals, seems very low. In such strains, expression of NAC2 does not require low Cu²⁺; however, when grown under natural light-dark cycles, the expression of psbD (and other chloroplast genes) is high during the day and low at night (17).

b. Normalized Expression of Cry4Aa₇₀₀—

Strains with normalized (i.e., psbD-like) expression of Cry4Aa₇₀₀ can be produced simply by transforming the psbD::Cry4Aa₇₀₀::psbA gene into the chloroplast of a wild-type (WT) strain, such as 137c (mt+). In the WT strain, NAC2 will stabilize the mRNA (without removing copper), and regulation of the transgene will be dominated by circadian (transcription) and diurnal (translation) rhythms (17). Chloroplast transformation, selection, and identification of homoplastic strains is contemplated to proceed as described herein. Then, the level and solubility of the Cry4Aa₇₀₀ protein will be determined, as described herein, with the change in that the cultures will be growing in normal (+Cu²⁺) medium and sampled at several points in the light-dark cycle. The larvicidal activity will be determined as described herein.

c. Truncation and Normalized Expression of Cry4Ba.

Strains with normalized expression of Cry4Ba will be generated with the same approach used to generate the normalized Cry4Aa₇₀₀ strains above, by transforming the psbD::Cry4Ba::psbA gene into a wild-type (mt+) strain. A shorter gene (i.e. truncated) and thus a smaller expressed protein is contemplated to improve Cry4Ba expression. Like Cry4Aa, the larvicidal activity of Cry4Ba is contained in the N-terminal half of the protein (5,6). Thus, we will generate a form containing amino acids 1-675, plus a small tag (HA) at the C-terminus to enable detection with a commercially available antibody.

The HA tag typically does not interfere with the protein's fold or function (9,37), and it contemplated to allow detection of Cry4Ba₆₇₅ in the presence of Cry4Aa₇₀₀ (which has the Flag tag). 5′ (psbD) and 3′ (psbA) expression signals used on the full-length protein will be used; the new gene will be referred to as psbD::Cry4Ba₆₋₇₅::psbA. Chloroplast transformation will be with a wild-type strain, and the transformants will be analyzed using the same methods employed on the Cry4Aa₇₀₀ transformants (herein), except the antibody will be for the HA tag.

III. Engineering Strains that Produce Combinations of Cyt1Aa(p20) and Cry Protoxins.

Combinations of two Cry genes and Cyt1Aa were the most effective at producing larvicidal cyanobacteria (44,46). Therefore, this combination is contemplated for expression in the Chlamydomonas chloroplast. Thus information, strains and constructs generated are contemplated to create combinatorial strains that express 2 of the Cry genes (e.g., Cry4Aa₇₀₀+Cry4Ba or Cry4Ba₆₇₅, Cry11Aa+Cry4Ba or Cry4Ba₆₇₅, etc.) and Cyt1A. In some embodiments, Cyt1A is expressed with Cyt1A-p20.

The inventors additionally contemplate using 2 locations in the chloroplast genome for transgene integration, with a particular gene orientation. In particular, avoiding creating direct repeats of the expression signals on the transgenes with those on the endogenous genes is desired (which could destabilize the genome). One location will be the same, between the psbA and rRNA genes, but the other site will depend, in part, on the gene(s) that will be integrated. Since this genome is 200,000 bp and has close to 100 genes, there are many intergenic locations that could work. Both transformations will rely on the aadA marker that can be recycled (15). This version is flanked by 500-bp direct repeats that recombine frequently enough to delete aadA from the genome, when the cells are grown without spectinomycin. This approach should enable the aadA marker to be recycled, and used repeatedly on the same transformants. The double-transformants will be re-streaked (to colonies) several times on spectinomycin, and then analyzed for transgene integration and homoplasmicity. Strains that have the correct DNA structure at both sites will be cultured and used for RNA and protein analysis (with the protein-specific antibodies), and in bioassays with larvae as described herein. For determining whether protoxins are associating or aggregating with each other, an antibody pull-down assays or by immunolocalization electron microscopy (12) will be used.

### IV. Contemplated Methods for Reducing Cry Toxicity in Algae.

The inventors also contemplate controlling the expression of Cry protoxins and Crt toxins at the protein level, rather than at the nucleic acid/gene expression level. One potential way CRY proteins are toxic to hosts is by interfering with chloroplast membrane function, thus keeping the Cry protoxin product away from the membranes during and after translation is contemplated to reduce toxicity.

Thus, the inventors further contemplate targeting genes encoding larvicides for localization in or near starch granules of algae, such as Chlamydomonas and other types of green algae. More specifically, a contemplated method is targeting the protoxins to starch granules using a starch-binding domain (SBD) for further reducing toxicity of expression of Cry proteins in algal. Thus expressed Cry protoxin could be localized to the starch grains by adding a starch-binding domain to the C-terminus (Ji et al., 2003). These are relatively small (˜100 amino acids) domains. As one example, adding an SBD is contemplated to reduce toxicity to Chlamydomonas without reducing expression of Cry4Aa₇₀₀ or resorting to inducible control, for e.g. reducing Cry4Aa₇₀₀ toxicity.

More specifically, starch-binding domains (SBDs) are contemplated for use to localize a protoxin (as a crystalline; intracellular inclusion) to the starch granules that surround the pyrenoid of the chloroplast (12). Binding protoxins to starch granules is contemplated to keep them away from the chloroplast membranes, which is where they might damage this organelle. Moreover, co-localizing protoxins to the starch surface might promote their association with each other, which may also have benefits for the host cell. Furthermore, starch is an excellent medium for stabilizing cells and proteins, in dehydrating conditions, such that the inventors contemplate additional benefits to host viability.

The most well studied starch-binding domains are bacterial, but they are also found in plants and Chlamydomonas. In fact, a nuclear-encoded enzyme for starch synthesis (GBSS) in Chlamydomonas was used recently to localize Plasmodium surface peptides to the starch granules in the chloroplast (12). The granule-bound enzyme is quite large (65 kD), however. Thus, the SBD from a Bacillus circulans cyclodextrin glycosyltransferase (19), which is ˜100 amino acids long is contemplated for use. Therefore, in one embodiment, DNA encoding the SBD is re-synthesized so that its codon usage will be a closer match to that of chloroplast genes, such as with the codon modified genes of the present inventions. This region will be used as a C-terminal fusion that is separated from the protoxin by a short linker (19). An epitope tag can also be added to the C-terminus when it is amplified for subcloning, or the SBD can be expressed in E. coli and used to elicit antibodies (19). In fact, the inventors designed a codon-optimized starch-binding domain using it to reduce Cry protein damage to the chloroplast.

This approach can be pursued in parallel with the development of the genetic controls, and will use many of the same materials and techniques. Localization of the protein in relation to starch granules may be determined by immunoelectron microscopy with specific antibodies, and by purifying the starch granules from the transformants and performing western blot analysis (12). Analyses on larvicidal activity will be performed on the corresponding non-SBD strains grown under the same conditions, in order to make meaningful comparisons. In one embodiment, SBD is contemplated to salvage a toxic protein-expression construct, e.g., one that inhibited growth in the inducible assay or that gave heteroplasmic transformants in the normalized expression assays.

Therefore, localizing the protoxins to starch granules is contemplated to eliminate, or significantly reduce, their potential to harm the host organelle. Thus in another embodiment, SBDs are contemplated for use in engineering strains that have higher levels of protoxins and for more potent combinations of protoxins.

V. A Strain of Wild-Type Chlamydomonas reinhardtii that is Constitutively Lethal to Mosquito Larvae: Cry11Aa Expression in the Chloroplast of Wild-Type Chlamydomonas.

Synthetic genes encoding mosquitocidal proteins Cry4Aa₇₀₀, Cry4Ba, and Cry11Aa were expressed in the chloroplast of an inducible Chlamydomonas reinhardtii strain, Ind41_18 as described herein. Inducible expression is useful for evaluating synthetic genes, when there is host toxicity. Moreover it is not always possible to predict which constructs or proteins will be toxic (Surzycki et al., 2009; Rasala and Mayfield, 2011). For practical reasons, growing Ind41_18 Chlamydomonas in waterways having controlled levels of copper is not feasible. Although numerous prokaryotes were engineered with Bti Cry transgenes, successes in eukaryotes involved yeasts, Saccharomyces cerevisiae and Pichia pastoris (Quintana-Castro et al., 2005; Borovsky et al., 2010). These transgenic yeast strains required carbon sources such as methanol, ethanol, or galactose for the induction of the Cry genes, making them unlikely to be useful in the field. Thus, constitutive expression or normalized expression in wild-type strains is desired for developing biolarvicides as living larvae food sources in waterways.

As described herein, each of the 3 cry synthetic genes having psbDₘ and psbA expression signals, were transformed into the chloroplast of a wild-type strain of C. reinhardtii. Homoplasmic Cry11Aa and Cry4Ba transformants were obtained but not Cry4Aa. These results show production of wild-type larvicidal-Chlamydomonas strains contemplated for mosquito control in water systems. Thus, larvicidal-Chlamydomonas strains can be used for mosquito control.

A. Summary of Wild-Type Transformants.

The successful development of a wild-type strain of C. reinhardtii that constitutively expresses Cry11Aa (i.e. without manipulations of the culture conditions) is described herein. This wild-type strain expressing novel Cry11Aa proteins is toxic to mosquito larvae (Aedes aegypti), see Example V. Chloroplast genes are expressed on a daily basis, mostly during the pre-dawn hours and throughout the daytime (Lee and Herrin, 2002; Misquitta and Herrin, 2005). Thus, each of the 3 synthetic novel genes described herein, were ligated in between plastid expression signals, i.e. psbaₘ (5′) and psbA (3′), then transformed (biolistically as described herein) into the chloroplast of a wild-type strain of C. reinhardtii. Homoplasmic (stable) Cry11Aa and Cry4Ba as separate (wt) transformants were obtained.

Western blotting confirmed the accumulation of Cry11Aa in the respective transformants, with a level that was at least as high as that obtained with the inducible Ind41_18 Chlamydomonas system. Lethality of the Cry11Aa^{WT} strain to Aedes aegypti larvae was confirmed with a live-cell bioassay. Further, the growth rate of the Cry11Aa^{WT} strain was indistinguishable from wild-type under standard growth conditions.

1. Cry11Aa.

Cry11Aa-producing strains were established with wild-type Chlamydomonas, in order to achieve a line constitutively toxic to mosquito larvae. PCR analysis confirmed the homoplasmicity of the chloroplast transformants. Western blotting showed that Cry11Aa of the expected size accumulated under standard growth conditions, and that the level was similar to that obtained in the inducible system. That is not surprising, perhaps, since the gene construct that was introduced into wild-type, psbaₘ:Cry11Aa.psbA, is the same as that used in the chloroplast of the inducible Ind41_18 strain. The lethality of the Cry11Aa-wt cells toward A. aegypti larvae was tested with the live cell bioassay, and found to be similar, or slightly less than that of the inducible strain grown under induction conditions. Incorporation of the psbaₘ:Cry11Aa.psbA gene into the wild-type chloroplast had no apparent detrimental effect on the growth of the cells, at least under our standard conditions. These results show that it is possible to generate C. reinhardtii strains that are constitutively toxic to mosquito larvae via chloroplast gene engineering.

In a further embodiment, Cry11Aa expression is increased by co-expressing the P20 chaperone from Bti. P20 is encoded on the pBtoxis plasmid in the Cry11Aa operon, and has been shown to specifically enhance the yield and crystallization of Cry4Aa, Cry11Aa, and Cyt1Aa via protein-protein interactions (Deng et al., 2014). Moreover, P20 alleviated the toxicity of Cyt1Aa to E. coli (Manasherob et al., 2001).

2. Cry4Ba.

Unlike Cry11Aa, Cry4Ba accumulation in the wild-type transformants was undetectable on the western blot. In the inducible strain, Cry4Ba accumulation was the lowest of the three Cry proteins, but it was still detectable. This result indicates that strain to strain variation in genetic background in C. reinhardtii can affect significantly the expression of an engineered Cry gene in the chloroplast. Perhaps by truncating Cry4Ba similar to Cry4Aa as shown herein, its expression might be improved in both systems, but given the lower toxicity of this protein to larva (Crickmore et al., 1995; Otieno-Ayayo et al., 2008), the increase in expression contemplated for a toxic effect might need to be similar to or greater than Cry11Aa.

3. Cry4Aa₇₀₀.

Putative Cry4Aa₇₀₀ wild-type transformants did not survive serial re-streaking on high spectinomycin suggesting that they could not reach high enough levels of spectinomycin-resistant ribosomes. Alternatively, the protein expressed from the novel Cry4Aa₇₀₀ gene of the present invention was toxic to the wild-type cells. However, Cry4Aa₇₀₀ accumulation in the inducible strain was substantially higher than Cry11Aa, so perhaps the wild-type strain is more susceptible. Alternatively, given the strain-dependent expression of Cry4Ba mentioned above, perhaps Cry4Aa₇₀₀ expression was higher in the wild-type background but was not sustainable. In comparison, the growth curve of the Cry4Aa₇₀₀ Ind41_18 transformant under inducing conditions was very similar to the growth curve under non-inducing conditions. However, compared to the growth of the wild-type strain it grows significantly slower and induction of the Cry4Aa₇₀₀ required removing Cu⁺² from the medium. Besides altering photosynthetic electron transport, Cu²⁺ starvation also alters the levels of >100 proteins in C. reinhardtii (Hsieh et al., 2013).

Thus the inventors' contemplate reducing Cry4Aa₇₀₀ toxicity to the wild-type strain of Chlamydomonas without reducing expression of Cry4Aa₇₀₀. It the toxicity is due to effects from the toxin associating with the chloroplast membranes, there are measures that can be taken to keep the Cry protein(s) away from them. Thus, translation of the Cry mRNA could be directed away from the membrane by replacing the 5′ UTR of psbD—which is translated on the thylakoid membrane (Herrin, et al., 1981)—with the 5′ UTR from the rbcL gene. RbcL mRNA is translated at the pyrenoid (Uniacke and Zerges, 2009). Second, the Cry protoxin could be localized to the starch grains by adding a starch-binding domain to the C-terminus (Ji et al., 2003). These are relatively small (˜100 amino acids) domains that would likely not interfere with protoxin processing and activity in the larvae. Third, the first and second suggestions could be combined, which should keep the Cry protoxin away from the membranes during and after translation.

B. Additional Wild-Type Transformants.

Further increases in Cry4Aa₇₀₀ toxicity are contemplated by co-expressing a P20 chaperone protein from Bti. P20 is encoded on the pBtoxis plasmid in the Cry11Aa operon, and has been shown to specifically enhance the yield and crystallization of Cry4Aa, Cry11Aa, and Cyt1Aa via protein-protein interactions (Deng et al., 2014). Moreover, P20 alleviated the toxicity of Cyt1Aa to E. coli (Manasherob et al., 2001). Thus in another embodiment, a Cry11Aa protein is expressed with a P20 protein.

### VI. Advantages of Using Larvicidal-Chlamydomonas of the Present Inventions.

In addition to advantages of using larvicidal-Chlamydomonas of the present inventions over other control measures, a Bti-modified food organism will also have an advantage over engineered mosquitoes which are being released as another approach to mosquito control. These engineered mosquitoes merely provide a measure of control for their own species. Whereas Bti-Chlamydomonas of the present inventions will provide control over numerous mosquito species. Therefore, inventions describe herein the discovery of compositions and methods during the development of a biological platform for mosquito control using as a host the eukaryotic green alga Chlamydomonas reinhardtii. These motile green algae were converted into a safe biolarvicide used as an edible alga capable of swimming and reproducing in aquatic habitats for use in mosquito control by reducing the number of viable mosquito larva in a water system. Thus, Chlamydomonas reinhardtii strains were engineered that are constitutively lethal to aquatic larvae due to the expression of unique versions of Bti proteins within the chloroplast. Also, these strains do not have bacterial antibiotic-resistance genes nor do they carry any natural bacterial sequences so they should be safer to other organisms in contact with this larvicide. In other words, these algae strains do not express additional toxins that are expressed by Bt and other bacteria. Moreover, Chlamydomonas reinhardtii strains can be engineered to target other pests, such as the fly ectoparasites that plague the cattle industry.

In particular, green algae grow and reproduce in larval host habitats (22). Thus, engineered larvicidal-green algae are contemplated to grow and reproduce these same larval habitats. During the motile flagella stage green alga are located in the water column (away from the bottom areas) as they swim around whereas in other life stages these alga sink to the bottom of the water. In particular, Chlamydomonas is edible and non-toxic to water organisms as a natural larval food source. The larval-destroying properties of the larvicidal strains of the present inventions are a different form of larvicides than the widely used compounds, i.e. Bti-larvicide, comprising the entire Bacillus thuringiensis ssp. israelensis (Bti) bacteria or concentrated crystals/protoxins. The use of Bti-larvicide has an excellent safety record.

A. Comparisons of the Use of Native Bti Toxins to Larvicides of the Present Inventions.

Although insect adulticides have a prominent place in emergency pest control, greater specificity (less damage to nontarget organisms) is achieved by employing larvicides. Bacillus thuringiensis ssp. israelensis (Bti) is used as a larvicide to help control mosquitoes in many parts of the world (4,8).

1. Use of Bti Bacteria and Isolated Bti Toxins.

Bti produces an internal parasporal toxin during sporulation that is highly specific for certain Dipterans (such as mosquitoes and black flies). The parasporal toxin is a crystal-like inclusion composed of at least 4 main proteins—three Cry proteins (Cry4A, Cry4B, Cry11A) and Cyt1A—that act synergistically to destroy the integrity of the gut membrane following ingestion by the larvae (10,42). Moreover, although Bti has been used for mosquito and black fly control for more than 25 years (4,18,40), there have been no cases of substantial resistance developing in target insect populations from field use (10).

Bti was reported to be toxic against larvae of 109 mosquito species; 40 species of Aedes, 27 species of Anopheles, and 19 species of Culex (Glare and O'Callaghan, 1998). Although Bti is toxic to a wide range of mosquito varieties, including those that are major disease vectors, the toxicity of specific protoxins varies significantly with the mosquito species. For example, Cry4Ba is highly toxic to Anopheles and Aedes, but weakly toxic to Culex spp., while Cry4Aa is highly active against Culex larvae. Cry11Aa is fairly lethal to all 3 genera. Cyt1Aa is weakly toxic to Aedes and Culex, and almost nontoxic to Anopheles (Frankenhuyzen, 2009, Poncet et al., 1995, Promdonkoy et al., 2005, Wu et al., 1994).

Synergism among the Bti toxins contributes to the low chance of development of resistance in mosquito larvae (Ben-Dov, 2014). The native Bti crystal is more toxic than any single or multiple-gene combinations (Poncet et al., 1995). Mixtures of Cry4Aa and Cry4Ba were 5-fold more toxic than Cry4A or Cry4Ba alone (Angsuthanasombat et al., 1992). Cyt1Aa dramatically (>5-fold) increased the toxicity of the Cry proteins, including Cr11Aa, presumably by acting as a receptor at the cell membrane (Wu et al., 1994; Poncet et al., 1995; Promdonkoy et al., 2005; Frankenhuyzen, 2009).

Bti was approved as a bio-mosquitocide by the US Environmental Protection Agency in 1981 (Becker, 2006), 5 years after its first isolation in Israel. Since then, Bti has been used around the world for the control of mosquitoes and black flies, and without a reported incident of highly resistant insects. For example, Bti application against black flies as part of the Onchocerciasis Control Programme (OCP) in West Africa rapidly reduced populations of this vector (Gullet et al., 1990). In Germany, mosquitoes of the Upper Rhine Valley were reduced by 90% from 1981 to 1991 by intensive Bti treatments, and there were no significant effects on the environment, as reported by Becker (1997).

Using Bti as a biocontol agent has several advantages over chemical pesticides. Bti is considered a safe mosquito control agent (WHO, 1999) because its toxicity is highly specific to Dipterans. No substantial toxicity has been detected in the field against non-Dipteran organisms, including other insects and invertebrates, fish, mammals and humans (Glare and O'Callaghan, 1998; Siegel, 2001). It is noted that chironomid midges were reported as being susceptible to the Bti toxin in a study of non-target organisms, but control of chironomid midges using Bti required seven-fold higher doses than for mosquitoes (Lacey and Merritt, 2003). When the Bti toxin was solubilized and injected at high doses into mice, some mortality was observed (Siegel and Shadduck, 1990). However, this toxicity by injection is not relevant to field applications, because the crystals are solubilized at alkaline pH, whereas the mammalian gut is acidic. Moreover, the toxin proteins are activated by proteases in the larval midgut, and the Cry proteins bind to specific receptors in the microvilli cell membrane (Margalit, 1989; Ben-Dov, 2014).

Another property of Bti that makes it attractive to use is that it does not induce strong resistance; several studies have reported no strong resistance of mosquito larvae to Bti crystals even after 30 years of application (Becker, 2000; Glare and O'Callaghan, 2000; Tetreau et al., 2013; Ben-Dov, 2014). Cyt1Aa in the PB suppresses resistance in mosquito larvae, as strong resistance to individual Cry proteins was detected in the laboratory and field (Tetreau et al., 2013; Ben-Dov, 2014).

More specifically, The Bti endotoxin can cause rapid mortality of target mosquito larvae. When the larvae were treated with the toxin, they stopped feeding within an hour, moved slowly within two hours, and became paralyzed by six hours (Chilcott et al., 1990). Bti toxin causes death of target mosquito larvae by forming pores in the cell membranes of midgut microvilli; thus, the mode of action is similar to that of toxins from other Bacillus thuringiensis species (Bravo et al., 2007). The 4 major proteins exhibit toxicity to varying degrees, however, Cyt1Aa also possesses cytolytic (and hemolytic) activity (Butko et al., 1996; Butko, 2003).

The Cry proteins are produced in a presumably inactive or protoxin form, while Cyt1Aa is produced in a partially active form. The Cry proteins are proteolytically activated in the insect gut while Cyt1Aa is also processed there to increase its activity (Chilcott and Ellar, 1988; Al-yahyaee and Ellar, 1995). The Cry protoxins are subjected to N-terminal and C-terminal processing, and intramolecular cleavage, leaving a three-domain structure that confers toxicity (Schnepf et al., 1998). Much of the C-terminal half, and 30-50 amino acids of the N-terminus of Cry4Aa and Cry4Ba are cleaved off, yielding activated forms with a size of ˜65 kDa (Ben-Dov, 2014). Further intramolecular cleavage produces two fragments, 20 and 45 kDa for Cry4Aa, and 18 and 45 kDa for Cry4Ba (Komano et al., 1998; Yamagiwa et al., 1999). For Cry11Aa, midgut proteases cleave off 28 residues at the N-terminus, and in the middle producing 34 and 32 kDa fragments (Dai and Gill, 1993) that remain associated with each other (Yamagiwa et al., 2004). Proteolytic cleavage of Cry4Aa and Cry11Aa probably involves trypsin, and for Cry4Ba, chymotrypsin (Yamagiwa et al., 2002; Xu et al., 2014).

The 28 kDa Cyt1Aa is also cleaved by midgut proteases at both termini, leaving a ˜25 kDa protein. Although it is a bacterial protease, proteinase K was reported to activate Cyt1Aa (Al-yahyaee and Ellar, 1995); the 24 kDa Cyt1Aa was approximately three times more effective than the protoxin (Butko et al., 1996). Also, the proteinase K-activated Cyt1Aa exhibited higher hemolytic activity than the trypsin-activated protein, owing to different cleavage sites of each enzyme (Al-yahyaee and Ellar, 1995).

Bti costs approximately 200 times less than a chemical insecticide (c.a. US$ 500,000 vs c.a. US$20 million) to develop and register (Becker and Margalit, 1993).

Although Bti is widely used, in whole and isolated form, it has limitations in addition to the ones described above. Although mosquitocidal products based on Bti are available on the open market and are used in many mosquito control programs, the use of the entire Bacillus thuringiensis ssp. israelensis (Bti) bacteria, or concentrated protoxins, has several drawbacks, including sensitivity to sunlight (UV light), sinking out of the water column leaving little to no toxin in the water column where many mosquito larvae are located, and a lack of recycling (Margalit, 1989; Myasnik et al., 2001). Also because it sinks to the bottom of the water column, it can be adsorbed by silt that lowers the accessibility of the toxin to mosquito larvae, including Anopheles, which are known to be surface feeders (Otieno-Ayayo et al., 2008).

Several early field tests reported that the toxicity of sporal cultures of Bti lasted less than 24 hours (Ramoska et al., 1982). However, the toxin in the silt retained its activity for 22 days, though most filter feeding larvae could not consume it (Ohana et al., 1987). Floating briquette formulations of Bti have been developed that slowly release the toxin and extend its persistence (Fansiri et al., 2006). Other additives protect the toxin from sunlight (Vilarinhos and Monnerat, 2004); UV in sunlight degrades tryptophan residues causing loss of its toxicity (Pusztai et al., 1991; Liu et al., 1993). Despite these advances, Bti still does not recycle in most aquatic environments.

The Bti bacterium also produces an exotoxin that is a water-soluble metabolite(s). The exotoxin is less specific than the crystal endotoxin and can damage non-target organisms like Trematode Cercariae (parasitic flatworms) (Horák et al., 1996). Commercial preparations of Bti have to be tested for the exotoxin and there is a tolerance level that must not be exceeded.

Hence, control with Bti requires frequent applications because of its short persistence in the areas where mosquito larvae are located. Also, Bti can produce other toxins (4,18) which cannot be present above certain specified levels in the commercial products.

To overcome some of these limitations of using isolated Bti toxins, there have been attempts to produce Bti-modified organisms (Bti-organisms) that express the protoxins and either, reproduce in larval habitats (aquatic bacteria) or provide an alternate source of the toxins (yeast) (Porter et al., 1993). Cry and/or Cyt1Aa genes were inserted into several gram-positive and gram-negative bacteria, including Bacillus subtilis (Ward et al., 1986), Ancylobacter aquaticus (Yap et al., 1994a), Caulobacter crescentus (Yap et al., 1994b), Pseudomonas putida (Xu et al., 2001), E. coli (Boonserm et al. 2004; Bukhari and Shakoori, 2009), and B. sphaericus (Federici et al., 2003). Also, several cyanobacterial species have been similarly engineered, including Agmenellum quadruplicatum, Synechocystis PCC 6803, Synechococcus PCC 7942, and Anabaena PCC 7120 (reviewed in Otieno-Ayayo et al., 2008). Cry protoxins have also been produced in two eukaryotic microorganisms: Cry11Aa was expressed in Saccharomyces cerevisiae, and Cry11Aa and a truncated Cry4Aa were expressed in Pichia pastoris (Quintana-Castro et al., 2005; Borovsky et al., 2010). However, as described above, the use of transgenic prokaryotes is not desirable.

A higher plant producing Cry11Aa in rice was made to provide resistant to bloodworms (Hughes, 2005). Since most insect pests of crops are not Dipterans, other classes of Cry toxins (such as Cry1A and Cry2A) derived from different subspecies of Bacillus thuringiensis for their toxicity to other insects were expressed in crop plants (Kleter et al., 2007).

2. Advantages of Using of Larvicides of the Present Inventions.

Therefore, the inventors contemplate overcoming these limitations by using motile algal strains as mosquito food sources expressing larvicides related to Bti toxins. These algae typically inhabit the water column where there would be greater contact of this novel larvicidal food source with the target larvae. Thus, motile algal strains would be engineered for more effectively controlling the numbers of mosquitoes that transmit disease, such as West Nile virus, dengue, encephalitis, malaria, etc., in a safe and sustainable manner.

Furthermore, the source of the Bti larvicides is not renewable, neither the bacillus added to the water nor the isolated crystals do not reproduce and thus does not last long in certain aquatic environments (24,29). Unlike the larvicidal-algae of the present inventions which would persist in larval habitats because its part of their natural habitat, thereby providing sustained control over time, i.e. over generations and seasonal changes of both algae and larvae lifecycles. Thus in one embodiment of the present inventions, the engineered strains of larvicidal-Chlamydomonas stains have the potential for sustained control of these insect pests. Additionally, Bti based larvicides are expensive to produce, and the additives in the commercial preparations can alter treated-habitats in undesirable ways. However, the inventors contemplate that the use of a host algal who's wild-type is naturally found in aquatic larval habitats would reduce undesirable side effects in treated areas. Moreover, in addition to reducing the use of chemical pesticides, amounts and number of applications, the use of the larvicidal strains of the present inventions would lower the cost of larvae control as compared to the cost of producing and using Bti larvicides which is relatively more expensive. In other words, algal strains of the present inventions are contemplated as easier and less expensive to produce than Bti based larvicides.

More specifically, the use of toxins related to Bti toxins in larvicidal Chlamydomonas has the advantage of that transgenes of the novel modified toxins described herein are less likely to be passed horizontally to other organisms in the environment. Unlike the genes obtained from classical mutants for the selection of transformants that originate from bacteria (references 17, 19, 25) the unique expression signals on the chloroplast-encoded genes of the present inventions related to photosynthesis typically do not express in bacteria or in the nucleus (28) of nonphotosyntetic organisms. Thus, reducing horizontal transfer and expression. The transmission of the novel genes of the present inventions to native Chlamydomonas strains in the field is contemplated by using Chlamydomonas in the minus (−) mating type where the chloroplast genome is inherited uniparentally from the plus (+) mating type (37). Thus the inventors contemplate numerous advantages of their larvicidal-green algae over the limitations on the use of Bti. Chemical pesticides are linked to serious non-target effects and eventually lose their effectiveness against their targets (due to the development of resistance).

B. Overcoming Limitations of Using Larvicidal-Algae of the Present Inventions.

The inventors contemplate that for some uses, the amount of larvicidal algae of the present inventions needed in algae population numbers is too high for a sustainable naturally growing larvicidal producing Chlamydomonas population within a water system. Further, a target larva would not ingest enough toxin within a regular ingested meal (amount) of larvicidal-Chlamydomonas. Therefore the inventors contemplate increasing the toxicity of the individual larvicidal algae or within the number of larvicidal Chlamydomonas consumed, so that fewer algal cells will be needed for a larvicidal effect. Thus, fewer larvicidal algae organisms or lower concentration of larvicidal-algae would be needed in order control (reduce) the number of mosquitoes. The inventors contemplate a benchmark (goal) of lethality to larvae at or below 1×10⁴ algal cells per ml of water habitat. Data acquired during the development of the present inventions using Cry4 shows 1×10⁵ algal cells per ml of water habitat. As reference, a mature culture of Chlamydomonas is ˜10⁷ cells/mL. The inventors contemplate achieving this goal, and simultaneously inhibiting the acquisition of resistance in the mosquitoes to Cry4, by co-expressing the Cyt1A protein (from Bti) from DNA designed for encoding a Cyt1A protein as described herein.

Even further, the inventors are contemplating generating strains of Chlamydomonas that are specific for controlling horn flies, which are major parasites of cattle. Horn flies, the most damaging of the cattle ectoparasites, cost the cattle industry about $1 billion a year in lost productivity. Therefore, a more effective manner of reducing the adult horn fly population is needed.

West Nile virus (WNV) has become endemic to the US, with yearly infection peaks coinciding with the activity of its mosquito vector. 2012 was the worst year for WNV since 2003 with 286 deaths and estimates of 86000-200000 non-neuroinvasive cases. There is no specific treatment or vaccine, so mosquito control is one approach to reduce disease transmission. Further, controlling the vector would also reduce the transmission of other diseases, such as Dengue. However, the chemical pesticides that have played a role in mosquito management are losing their effectiveness due to increasing resistance. Moreover, there are growing concerns over damage to non-target organisms (such as honeybees), and possible links between pesticide exposure and neurogenerative diseases in people. We are developing new products for mosquito control that are based on the simple idea of turning a larval food source, the eukaryotic green alga Chlamydomonas, into a safe and effective biolarvicides. Chlamydomonas is an edible alga whose ability to swim and reproduce in aquatic habitats, and its development as a genetic research model make it an attractive platform for mosquito control. To this end, the inventors contemplate expressing genes based on the Cry and Cyt genes of Bacillus thuringiensis ssp. israelensis (Bti) in the chloroplast of Chlamydomonas. Bti is a natural biolarvicide that has not produced strong resistance in >20 yrs of use, but it does not recycle in aquatic habitats. In addition, by using the chloroplast genome with codon-modified genes and without bacterial antibiotic-resistance genes, the possible transfer of Bti genes to other organisms is greatly reduced. The inventors contemplate creating and producing robust strains that express 1 or 2 Cry genes and Cyt1Aa, which inhibits the development of strong resistance. Since we have demonstrated recently that Cry genes can be expressed in the organelle, our priorities for Phase I are to express Cyt1Aa in the chloroplast, and to identify elements that increase Cry gene expression using Cry11Aa as a model.

VII. Contemplative Adaptation of Protoxin Genes from Other Organisms for Use in the Algal Food Organisms of the Present Inventions.

The following briefly describes exemplary genes encoding protoxins and toxins from organisms other than Bti, along with their translated proteins, that are contemplated for modification and use in algal organisms of the present inventions. In some embodiments, larvicidal-Chlamydomonas of the present inventions may additionally express one or more of the exemplary genes described below for producing additional larvicidal-Chlamydomonas strains. In some embodiments, additional larvicidal-Chlamydomonas strains may be toxic to mosquitoes and other disease causing insect larvae.

A. Toxins of Additional B. thuringiensis Subspecies and their ProToxins.

Bacillus thuringiensis has numerous subspecies producing additional Cry protoxins, Bacillus sphaericus produces a binary toxin, and Clostridium species produce Cry toxins, as described below, which may find use in producing additional larvicidal Chlamydomonas strains engineered for safe use in and around humans.

1. B. thuringiensis jegathesan produces at least 8 protoxins: Cry11Ba (81 kDa), Cry19Aa (75 kDa), Cyt2Bb (30 kDa), Cry24Aa (76 kDa), Cry25Aa (76 kDa), Cry30Ca (77 kDa), Cry60Aa (34 kDa), and Cry60Ba (35 kDa) (Sun et al., 2013). Several of these proteins are immunologically related to the protoxins of Bti, including Cry11Ba, which is related to Cry11Aa, and Cyt2Bb, which is related to Cyt1Aa (Delécluse et al. 1995, Delécluse et al., 2000). Among the Cry proteins, Cry11Ba exhibited the strongest toxicity against mosquito species Aedes aegypti (A. aegypti), Culex pipiens, and Anopheles stephensii (Delécluse et al., 1995). Cry19Aa was toxic to Culex pipiens, Culex quinquefasciatus (C. quinquefasciatus), and Anopheles stephensii, but weakly to A. aegypti (Rosso and Delécluse, 1997). ORF2 in jegathesan promotes stability and crystallization of other Cry proteins, thus increasing toxicity (Sun et al., 2013), thus is contemplated for use in some embodiments of the present inventions.

2. B. thuringiensis medellin also produces several Cry proteins: Cry11Bb (94 kDa), Cry29Aa (74 kDA), Cry30Aa (78 kDa), Cyt1Ab (28 kDa), and Cyt2Ba (30 kDa) Delécluse et al., 2000). Cry29Aa and Cry30Aa exhibited no activity, but Cry11Bb, Cyt1Ab, and Cyt2Ba were toxic to mosquito larvae. Cry11Bb exhibited high toxicity against A. aegypti, Anopheles albimanus and C. quinquefasciatus larvae (Orduz, 1998).

B. Bacillus sphaericus Toxins.

Bacillus sphaericus (Bs) is a sporulating, aerobic, gram-positive soil bacterium (El-Bendary, 2006) that has also been employed for mosquito control since the late 1980s (Poopath and Abidha, 2010). The first mosquitocidal Bs strain, neide, was isolated from carcasses of mosquito larvae near Fresno, Calif. in 1965 (Kellen et al., 1965); thereafter, hundreds of Bs strains were identified.

Bs produces several mosquitocidal toxins, with a major binary toxin produced in strains 1593 and 2362 (Pena-Montenegro and Dussan, 2013; Silva-Filha et al., 2004). During sporulation, Bs produces a parasporal body that contains the binary toxin, which is composed of BinA (42 kDa) and BinB (51 kDa). Its mode of action is similar to that of the Bti toxin (Poopathi and Abidha, 2010). Upon ingestion by larvae, the heterodimeric toxin is cleaved by proteases into active 39 kDa (BinA) and 43 kDa (BinB) proteins (Baumann et al., 1991; Canan, 2013), which act synergistically (Arapinis et al., 1988; Nicolas et al., 1993). Equal amounts of BinA and BinB provide maximum activity, and BinB is required for the activity of BinA (Baumann et al., 1991). BinB binds to a specific receptor, which is a 60-kDa α-glucosidase in Culex pipiens, while BinA is involved in conferring toxicity (Darboux et al., 2001). The toxin is thought to participate in pore formation in the larval midgut (Schwartz et al., 2001). Bs has no reported toxicity against non-target organisms, including fish, mice, and humans (Shadduck et al., 1980; Grisolia et al., 2009; Oliveira-Filho et al, 2014).

The Bs binary toxin has some properties that are different from Bti crystals. The host range of Bs is more restrictive; it has high toxicity against Culex, but not A. aegypti or black flies (Wraight et al., 1987; Berry et al., 1993). Bs also acts more slowly than Bti (de Barjac, 1989), but the toxicity persists for longer periods and it can recycle in the field (Nicolas et al., 1987; Pantuwatana et al., 1989). The Bs toxin is also effective in polluted water, unlike Bti (Baumann et al., 1991; Wirth et al., 2010); however, it does engender resistance (Poopathi and Abidha, 2010). The resistance of Culex mosquito larvae to the Bs toxin has been reported in laboratory and field conditions (Silva-Filha et al., 1995; Wirth et al., 2000; Amorim et al., 2007). The main cause of this resistance seems related to the fact that, compared to Bti, it has a single major toxin with a relatively simple mode of action (Nielsen-Leroux et al., 1995). Co-expression of Cyt1Aa from Bti with the binary toxin has improved toxicity to resistant Culex larvae (Park et al., 2005; Wirth et al., 2010). Thus, in some embodiments, toxin genes BinA and BinB are co-expressed with a novel Cyt1Aa gene of the present inventions in Chlamydomonas. In some embodiments, toxin genes BinA and BinB are co-expressed with novel cry genes of the present inventions in Chlamydomonas.

C. Clostridium Toxin.

Clostridium bifermentrans malasya, which produces Cry16A and Cry17A in addition to other toxins, is an anaerobic, non-B. thuringiensis organism expressing Cry proteins (Barloy et al., 1996). Cry16A and Cry17A were weakly toxic to Anopheles, Aedes, and Culex mosquito larvae (Qureshi et al., 2014). Thus, in some embodiments, Cry16A and Cry17A are co-expressed with a novel Cyt1Aa gene of the present inventions in Chlamydomonas. In some embodiments, Cry16A and Cry17A genes are co-expressed with a cry11Aa gene of the present inventions in Chlamydomonas.

D. The Following are Exemplary Sequences for Bti Cry Genes.

National Center for Biotechnology Information (NCBI) cry11AA Pesticidial crystal protein cry11AA [Bacillus thuringiensis serovar israelensis] Gene ID: 5759849, updated on 21 Oct. 2014. Location: plasmid: pBtoxis. >gi∥161598544|ref|NC_010076.1|:19497-22008 Bacillus thuringiensis serovar israelensis plasmid pBtoxis, complete sequence: 2512 nucleotides:

AGAAAACATTGCTGTGAGTTGCCAAGATACTGTCTGCGTAGATCAAGTTT
TGTATTGCAGTGTAGATTGTTTGCCAGATTGTGATATTAATTGTGATAAT
GTAAAAATTTGCGATGTGAGCATTGAACCAATTGGAGATTGTGATTGTCA
CGCGGTGAAAATTAAAGGGAAATTTTCACTTCACTATAAATAAAAAATCC
CTAATTATTAAATGAATAATAAGGTCATAATTTATGAATAAAAATATGAC
CTTTAAAATAAAAAAATTCAATAAAAGGTGGAATGAATTATATGGAAGAT
AGTTCTTTAGATACTTTAAGTATAGTTAATGAAACAGACTTTCCATTATA
TAATAATTATACCGAACCTACTATTGCGCCAGCATTAATAGCAGTAGCTC
CCATCGCACAATATCTTGCAACAGCTATAGGGAAATGGGCGGCAAAGGCA
GCATTTTCAAAAGTACTATCACTTATATTCCCAGGTTCTCAACCTGCTAC
TATGGAAAAAGTTCGTACAGAAGTGGAAACACTTATAAATCAAAAATTAA
GCCAAGATCGAGTCAATATATTAAACGCAGAATATAGGGGGATTATTGAG
GTTAGTGATGTATTTGATGCGTATATTAAACAACCAGGTTTTACCCCTGC
AACAGCCAAGGGTTATTTTCTAAATCTAAGTGGTGCTATAATACAACGAT
TACCTCAATTTGAGGTTCAAACATATGAAGGAGTATCTATAGCACTTTTT
ACTCAAATGTGTACACTTCATTTAACTTTATTAAAAGACGGAATCCTAGC
AGGGAGTGCATGGGGATTTACTCAAGCTGATGTAGATTCATTTATAAAAT
TATTTAATCAAAAAGTATTAGATTACAGGACCAGATTAATGAGAATGTAC
ACAGAAGAGTTCGGAAGATTGTGTAAAGTCAGTCTTAAAGATGGATTGAC
GTTCCGGAATATGTGTAATTTATATGTGTTTCCATTTGCTGAAGCCTGGT
CTTTAATGAGATATGAAGGATTAAAATTACAAAGCTCTCTATCATTATGG
GATTATGTTGGTGTCTCAATTCCTGTAAATTATAATGAATGGGGAGGACT
AGTTTATAAGTTATTAATGGGGGAAGTTAATCAAAGATTAACAACTGTTA
AATTTAATTATTCTTTCACTAATGAACCAGCTGATATACCAGCAAGAGAA
AATATTCGTGGCGTCCATCCTATATACGATCCTAGTTCTGGGCTTACAGG
ATGGATAGGAAACGGAAGAACAAACAATTTTAATTTTGCTGATAACAATG
GCAATGAAATTATGGAAGTTAGAACACAAACTTTTTATCAAAATCCAAAT
AATGAGCCTATAGCGCCTAGAGATATTATAAATCAAATTTTAACTGCGCC
AGCACCAGCAGACCTATTTTTTAAAAATGCAGATATAAATGTAAAGTTCA
CACAGTGGTTTCAGTCTACTCTATATGGGTGGAACATTAAACTCGGTACA
CAAACGGTTTTAAGTAGTAGAACCGGAACAATACCACCAAATTATTTAGC
ATATGATGGATATTATATTCGTGCTATTTCAGCTTGCCCAAGAGGAGTCT
CACTTGCATATAATCACGATCTTACAACACTAACATATAATAGAATAGAG
TATGATTCACCTACTACAGAAAATATTATTGTAGGGTTTGCACCAGATAA
TACTAAGGACTTTTATTCTAAAAAATCTCACTATTTAAGTGAAACGAATG
ATAGTTATGTAATTCCTGCTCTGCAATTTGCTGAAGTTTCAGATAGATCA
TTTTTAGAAGATACGCCAGATCAAGCAACAGACGGCAGTATTAAATTTGC
ACGTACTTTCATTAGTAATGAAGCTAAGTACTCTATTAGACTAAACACCG
GGTTTAATACGGCAACTAGATATAAATTAATTATCAGGGTAAGAGTACCT
TATCGCTTACCTGCTGGAATACGGGTACAATCTCAGAATTCGGGAAATAA
TAGAATGCTAGGCAGTTTTACTGCAAATGCTAATCCAGAATGGGTGGATT
TTGTCACAGATGCATTTACATTTAACGATTTAGGGATTACAACTTCAAGT
ACAAATGCTTTATTTAGTATTTCTTCAGATAGTTTAAATTCTGGAGAAGA
GTGGTATTTATCGCAGTTGTTTTTAGTAAAAGAATCGGCCTTTACGACGC
AAATTAATCCGTTACTAAAGTAGAAGTCATGTTAGCACAAGAGGAGTGAG
TATTGTGGCTCCTCTTGTAATTTTAATCGCTAATATTTCTAATAGATATA
AATTATATATAATATTTAAAAAGTTATAATTATGTAATTGTAGAAAATCA
TGAATTTTTCAATTTTATTGACGAGGAAACAGAGTATACGAGTTTATAAT
TTCTAATAATTGTTTAAAACATATGCTTAGAAGTCAATTTATATTAGCTT
TACTTTTAGTAGAATTTATAATTAATATTTAGGATAAAATTGGAGGATAA
TTGATGACAGAA

SEQ ID NO:01. A codon modified Cry11Aa: 1938 nucleotides. 76% identical over 86% of the sequence.

ATGCTCGATATGGAAGACTCATCATTAGACACTTTATCAATTGTAAACGAAA
CTGACTTCCCATTATACAACAACTACACTGAACCAACTATTGCTCCAGCTTT
AATTGCTGTAGCTCCAATTGCTCAATACTTAGCTACTGCTATTGGTAAATGG
GCTGCTAAAGCTGCTTTCTCAAAAGTATTATCATTAATTTTCCCAGGTTCAC
AACCAGCTACTATGGAAAAAGTACGTACTGAAGTAGAAACTTTAATTAACCA
AAAATTATCACAAGACCGTGTAAACATTTTAAACGCTGAATACCGTGGTATT
ATTGAAGTATCAGACGTATTCGACGCTTACATTAAACAACCAGGTTTCACTC
CAGCTACTGCTAAAGGTTACTTCTTAAACTTATCAGGTGCTATTATTCAACG
TTTACCACAATTCGAAGTACAAACTTACGAAGGTGTATCAATTGCTTTATTC
ACTCAAATGTGTACTTTACACTTAACTTTATTAAAAGACGGTATTTTAGCTG
GTTCAGCTTGGGGTTTCACTCAAGCTGACGTAGACTCATTCATTAAATTATT
CAACCAAAAAGTATTAGACTACCGTACTCGTTTAATGCGTATGTACACTGAA
GAATTCGGTCGTTTATGTAAAGTATCATTAAAAGACGGTTTAACTTTCCGTA
ACATGTGTAACTTATACGTATTCCCATTCGCTGAAGCTTGGTCATTAATGCG
TTACGAAGGTTTAAAATTACAATCATCATTATCATTATGGGACTACGTAGGT
GTATCAATTCCAGTAAACTACAACGAATGGGGTGGTTTAGTATACAAATTAT
TAATGGGTGAAGTAAACCAACGTTTAACTACTGTAAAATTCAACTACTCATT
CACTAACGAACCAGCTGACATTCCAGCTCGTGAAAACATTCGTGGTGTACAC
CCAATTTACGACCCATCATCAGGTTTAACTGGTTGGATTGGTAACGGTCGTA
CTAACAACTTCAACTTCGCTGACAACAACGGTAACGAAATTATGGAAGTACG
TACTCAAACTTTCTACCAAAACCCAAACAACGAACCAATTGCTCCACGTGAC
ATTATTAACCAAATTTTAACTGCTCCAGCTCCAGCTGACTTATTCTTCAAAA
ACGCTGACATTAACGTAAAATTCACTCAATGGTTCCAATCAACTTTATACGG
TTGGAACATTAAATTAGGTACTCAAACTGTATTATCATCACGTACTGGTACT
ATTCCACCAAACTACTTAGCTTACGACGGTTACTACATTCGTGCTATTTCAG
CTTGTCCACGTGGTGTATCATTAGCTTACAACCACGACTTAACTACTTTAAC
TTACAACCGTATTGAATACGACTCACCAACTACTGAAAACATTATTGTAGGT
CTTCGCTCCAGACAACACTAAAGATTCTACTCAAAAAAATCACACTACTTAT
CAGAAACTAACGACTCATACGTAATTCCAGCTTTACAATTCGCTGAAGTATC
AGACCGTTCATTCTTAGAAGACACTCCAGACCAAGCTACTGACGGTTCAATT
AAATTCGCTCGTACTTTCATTTCAAACGAAGCTAAATACTCAATTCGTTTAA
ACACTGGTTTCAACACTGCTACTCGTTACAAATTAATTATTCGTGTACGTGT
ACCATACCGTTTACCAGCTGGTATTCGTGTACAATCACAAAACTCAGGTAAC
AACCGTATGTTAGGTTCATTCACTGCTAACGCTAACCCAGAATGGGTAGACT
TCGTAACTGACGCTTTCACTTTCAACGACTTAGGTATTACTACTTCATCAAC
TAACGCTTTATTCTCAATTTCATCAGACTCATTAAACTCAGGTGAAGAATGG
TACTTATCACAATTATTCTTAGTAAAAGAATCAGCTTTCACTACTCAAATTA
ACCCATTATTAAAA

National Center for Biotechnology Information (NCBI) pesticidial crystal protein cry4AA [Bacillus thuringiensis serovar israelensis] Gene ID: 5759905, updated on 21 Oct. 2014. >gi|161598544|ref|NC_010076.1|:92454-97058 Bacillus thuringiensis serovar israelensis plasmid pBtoxis, complete sequence: 4605 nucleotides (nts). Gene starting at 1977.

TATTTTTTTATTATGTACGAAAAAAAGCATTCATCTTTCAAGTAGATGAATGC
AAAAATTAATTTGAAATTTAATGTATTTTTATAAGTGGCCCCAAAAAGAAGG
AATCGTTGCCGTGCCCCCTGTACAGGCAGAACCACAATCTGATAAAGGACTC
CATGGAAATTGAGGATCGGAGGTAATCGCAAAAGCTCGAAGTATTAAGATTT
GAAATCGATTGTTGATTTCCCTGCATATTCTTTCCCTCATTTTGTTTGATGAAA
ATCTATTTTCAAATCCTAAATCAGTTCATCTATTAATCATCATAACTTGGATC
ACAATTGTAGTTTGGATAGTTTAAATGGTGATAATTATTATTGGATAAACGTT
CTATACTAATGAAATTGATATTTGTATAAATTTTATGTCCTCTAGATATCTATT
TTTTATGTTTTCTATATATTTTTTGTACCAGAATTAATAAATGCAGAAATTAAA
AACCATGGAGAAAACTTTCTCCATGGTTTTTAAAGCTTTAGTTATTTTTTATTA
ATCACTCGTTCATGCAAATTAATTCAATGCTTTCGATATAAAACGAACCTTCG
GTTTCGCCTATCTCAATTCGTACACGATCTGTATCTGGGAATACATCTACTGT
CTTCGTAATATATCCTTCTTCACAAGACGTAAACGTCAATTTTTCTTGATTCTC
CTCACAATCCATAAGCGTGACATACCCATTTCCAGGTCCTTCTTTTTTGGCAA
TAACACGTAAGACATACCCATGATTATGTTGGAGATGGACATTTTGAGATAC
GCCAGCACTCCAATTAGATAGAACCAATACAGAAACACCATCTATTTGTTGT
ACGTCTGCATTTCCAGTTACATGCCACCCCATTACCCCTTGTGTAAAATCACC
ATTTTTAATAATATTTCTTGTATCATACAAATAACGCGCTTGTGCCACTCGTG
CATCCAACTCTACATAGATATCATAATTCATACCTGGAACATCTGACAACCAA
TCATTGTACACATATGGAATCGATTGTACCAAATACTCAGCGTACTGAATTTG
AGCGAGTGTCGTATCAAACTGTAAAGCCTCATCTTGTACATTTGTGAATAAAG
CATCAATGGCTTGTTTCGCTACATCATATGCTTGTTGTGTTTCCGAACGTTTTG
CTTCCATTTGATCGTTCCATTTCTTCTCCATGTGTTTCACGCGTGACAGTGCTT
CCCCATCTATTGGCCCTTCTTCAATTACTTCTAAATTATCTAATGATGCGTATC
CATCTGGAGAAGATATTTTAAACATGACCCAAACCCCTATATTTTCATTTGTA
TCTAATGCCCCTGTATCAATAGTGAAACTAAATTGATGGGAATCCTGACATAC
GACATGCTTTTTCCCTGTATCATATTGGCATGAATACAACATATCAGAAGTGT
TCCCAATGTTAGCCGGCACAGCGGACGTCTCACAACGATTAGACCCTTCACA
ATCAAAGGTAGAAGGATACAGATAGTTTAAATCAGCTGGAACATTCATGATG
GCATCAATTTCTTCCCCATAGCGTGAAACCACTAGTTCTACATCTTTACTACTT
CCTACAAATCCCCTTACTAGGTAACGTGTATACGGTTTTAATTTTGATTCATC
AATTTTTTGGAATATATAGGTCGGAAATATCGTACCATCAATGTCTCTCGCCC
CAGACATATGAAGGTAATGCCCTTTAAAAATAGGATCATCTTCTTGAATTGTG
ATATTATCACTTGTTGTCCAACCAAGCGTAGCCGATTCAAAATCCCCGTTTTG
AAGTACATTTCGAGATTGACTAAGTTGTTTCGCATTTTTAACTTCATCTAATA
ACAGCATTTTTTCTTTTGGATATAATTCTTCAGAAATACATTCCACAAGATTT
GCGGCTTGATCTATGTCATAATCTGTAAGTTCTGATTGTAAAGTGTTTTTTATA
GGATTTGCATAA: gene start:
AATGTATTAATTATTTGTTGTACTGTTTCTAATTTTTGTTTCTCTCTATCCTCTC
TTATAGAACGAGTAATTGGCAGAAATTCAATTTTATCAATAAGTACTGTTGTG
TTTGTATATACATCCGAACGATTAAACACAAGAGATATGTTTTGATTTGGAGC
AAATTTCACCTCGTTAGAAAATTCTAAGTACTGAAAATCTTTATATTTTAAAT
TCGTATAATCTGTACCAGAAAAAGTGGGGTTGAGTGCCATACCCAGTTCTGCT
ACCCCTGGGATACTAAGATTTATAACAGCTCGAGTATTTGCGCTTCCATTTGA
AGCATAACGAATTCTTATAAAATACGATTGTTGAAAATTTGAGTGTTGACATG
TAATTTTGAAATGATCTTTGAAATCAATTAAATCCCCTCCTGTATGACCAGGT
CCTTGAACAACCTTAGAAGCAGTCCCAAGTGAATTCGCTTTTACAGCTGGAAT
TTGGGTAGTTAAATGTGTATAAATTGTATTTTTAGGATCAACACTAGAGTGTG
TCCAAGCAAACGTATACACTTGAGTTTTATATGTTGCAGGGATACTAAGACTT
TTAATAAATGATAAAATATGACTATAGTTATCATATGTTGGAAAAAGGGTAG
GGTTTCCTTGATTCTCTCTTCGTTTAAGAATTGGTAACCCGAAAATATTTTTAT
TTACATCATAAGTTATTTGCCCAGATCCTGCTGTAAGTTCTTTCTCCAAAAGT
CTAGTACCATTAGTTATAAAAAAATCCATTTTACTAATATTATTATAATCATT
TAGATATTTATTATCTAAGCTTATGACATTTAATAAAAAAATATAAATATTTG
TTGCCAAACCAAGAGATTTTAATTTATCAGTTACATTGTGATTTCCAAAAACA
CTAGATTTTTGGGATATATTATCAAGTGTGTAATGAAACATATTATAATGGCT
GGTGAAAAAATTATTAGGAGTAGTTTGCGCTTTTTCATAAAAATTCAAAGAA
TCAAGCCAAGTAAATAAATGCGGTCTACGTGTAAGTGAATCCTCTTGATATTG
AAAGTCATAATATTTATAGGGGCTTTCTTCGAAGTTAAGTACCTGATAAATTT
CTCGAGTAAGTTCAGATTGGACACCTATTGGATATTTACCTACATCATAATTA
GGAAAGAGTGCAACAAGATCTAATACAGCAGTAGTCATTTTTGTTCGATACG
TATTGTATGTGTTCCAGTTTATATTTCCATCAAGATTACTATCAGGCGTCGTTT
TAATTAAATTTAATCCTTTTTTATAAGTTGTTACACAATAATTAGTGTAATCTT
CTATAGCTTTAGTCAATACTGGATAATAATCAATTGCTGTTGGCAAAGGCTCT
AAATAATCGAATTGTCGATTGTTTTTTAAATACGCTTCAAATTTGACGGCTTG
ATTTAATACAGTCAGATGTAAGTTTGCTGCTTGTGCATAACTAGATAATACTA
GTATGTTATAGTAATCGCAATCACTAGGATTAGGAGGACAAGAGTTTACAAG
CTCTGGAATGACATTTTGAAAATGGTAATGAACTAGCTGGATTTGTGTCCTTA
CATCCTGAGTATTTTGTGGGTTTGGATTATTCTCCCATGTTTTAAGGTGATTAT
GATAAGTGCTGATAACATTAAACGACCTGTTTAAAATTTTATTAGCATTACTT
ATATATGTTGATGCTATTTCTTTTTTTATAATATTTTTAGTTTGTGTTATAAAG
TCACTCCATGTGTTAGATTGGTCTTGGGCTGGAAAAAGAACTGGTATTAATGT
ACCAAAACCTATTAAAGCAAGTCCTAAGGGTGTTGTGAACCCGAAACCAGTC
AGTACGGTCCCAACTACAATAGTATAGGCACTGAGTTCACCACTATCAATAA
AAGTTTCAAAATCTCCACCATACTGCTGATTCTGTTGACACATATTGAGCCAA
TCTTTATAATTTGTACTTTGTAATAATTGTTTTGGACTATTTTCTATTGGATAT
CTTGTATAATTATTAGATATATTTAATTTTTTTTGTGAAGCATTTAATGTTTCA
TATTCATTTTTATTTTGATAAGGATTCATATTTGTTCCTCCCATACTCAATTTA
GATACACTCTTTTTCTGTAGCAACAAAGATTATTTTAAATCATTTTTAAATTG
ATATGGTTTAAAAAGTACAAAATTGAAAATTATTGATTACTTTTACAAATCCT
ATATACATATTAATGTACCAATATAATTATTCGTAATTTATACATTTTAAAAA
TTTTTGCGTTAAATTTTTAAAACTTTGTATTTCATATGGTTTGTTACAAAACCT
CACACAAAAATAAGAAAACCTTCTTTACAAGAATTCTTGGTATCTTTGACCCT
TATGCATTTATCCTCTCCTATGTAGTAATCTCTCTTTCTTTTACACTCCAAGCT
ATCAAAATTTCCCTTATGCATTTTAAAGTATTCGTAATTTAAATAATCTATTCC
TGTTACATTCTTTCAACAAATAACCGCGTCATTTTTTGACAATCAACCAGCCT
GTTAATTTTTAAAAAAGCTATCTAATCCCCTTCAATATCCCTTTATATGCCTTT
TACATCAAATAGTATAGGAACTGA

SEQ ID NO:02. A condon modified Cry4Aa: 2100 nucleotides. 77% identical over 79% of the sequence.

ATGAACCCATACCAAAACAAAAACGAATACGAAACTTTAAACGCTTCACAAA
AAAAATTAAACATTTCAAACAACTACACTCGTTACCCAATTGAAAACTCACC
AAAACAATTATTACAATCAACTAACTACAAAGACTGGTTAAACATGTGTCAA
CAAAACCAACAATACGGTGGTGACTTCGAAACTTTCATTGACTCAGGTGAAT
TATCAGCTTACACTATTGTAGTAGGTACTGTATTAACTGGTTTCGGTTTCAC
TACTCCATTAGGTTTAGCTTTAATTGGTTTCGGTACTTTAATTCCAGTATTA
TTCCCAGCTCAAGACCAATCAAACACTTGGTCAGACTTCATTACTCAAACTA
AAAACATTATTAAAAAAGAAATTGCTTCAACTTACATTTCAAACGCTAACAA
AATTTTAAACCGTTCATTCAACGTAATTTCAACTTACCACAACCACTTAAAA
ACTTGGGAAAACAACCCAAACCCACAAAACACTCAAGACGTACGTACTCAAA
TTCAATTAGTACACTACCACTTCCAAAACGTAATTCCAGAATTAGTAAACTC
ATGTCCACCAAACCCATCAGACTGTGACTACTACAACATTTTAGTATTATCA
TCATACGCTCAAGCTGCTAACTTACACTTAACTGTATTAAACCAAGCTGTAA
AATTCGAAGCTTACTTAAAAAACAACCGTCAATTCGACTACTTAGAACCATT
ACCAACTGCTATTGACTACTACCCAGTATTAACTAAAGCTATTGAAGACTAC
ACTAACTACTGTGTAACTACTTACAAAAAAGGTTTAAACTTAATTAAAACTA
CTCCAGACTCAAACTTAGACGGTAACATTAACTGGAACACTTACAACACTTA
CCGTACTAAAATGACTACTGCTGTATTAGACTTAGTAGCTTTATTCCCAAAC
TACGACGTAGGTAAATACCCAATTGGTGTACAATCAGAATTAACTCGTGAAA
TTTACCAAGTATTAAACTTCGAAGAATCACCATACAAATACTACGACTTCCA
ATACCAAGAAGACTCATTAACTCGTCGTCCACACTTATTCACTTGGTTAGAC
TCATTAAACTTCTACGAAAAAGCTCAAACTACTCCAAACAACTTCTTCACTT
CACACTACAACATGTTCCACTACACTTTAGACAACATTTCACAAAAATCATC
AGTATTCGGTAACCACAACGTAACTGACAAATTAAAATCATTAGGTTTAGCT
ACTAACATTTACATTTTCTTATTAAACGTAATTTCATTAGACAACAAATACT
TAAACGACTACAACAACATTTCAAAAATGGACTTCTTCATTACTAACGGTAC
TCGTTTATTAGAAAAAGAATTAACTGCTGGTTCAGGTCAAATTACTTACGAC
GTAAACAAAAACATTTTCGGTTTACCAATTTTAAAACGTCGTGAAAACCAAG
GTAACCCAACTTTATTCCCAACTTACGACAACTACTCACACATTTTATCATT
CATTAAATCATTATCAATTCCAGCTACTTACAAAACTCAAGTATACACTTTC
GCTTGGACTCACTCATCAGTAGACCCAAAAAACACTATTTACACTCACTTAA
CTACTCAAATTCCAGCTGTAAAAGCTAACTCATTAGGTACTGCTTCAAAAGT
AGTACAAGGTCCAGGTCACACTGGTGGTGACTTAATTGACTTCAAAGACCAC
TTCAAAATTACTTGTCAACACTCAAACTTCCAACAATCATACTTCATTCGTA
TTCGTTACGCTTCAAACGGTTCAGCTAACACTCGTGCTGTAATTAACTTATC
AATTCCAGGTGTAGCTGAATTAGGTATGGCTTTAAACCCAACTTTCTCAGGT
ACTGACTACACTAACTTAAAATACAAAGACTTCCAATACTTAGAATTCTCAA
ACGAAGTAAAATTCGCTCCAAACCAAAACATTTCATTAGTATTCAACCGTTC
AGACGTATACACTAACACTACTGTATTAATTGACAAAATTGAATTCTTACCA
ATTACTCGTTCAATTCGTGAAGACCGTGAAAAACAAAAATTAGAAACTGTAC
AACAAATTATTAACACTTTC

National Center for Biotechnology Information (NCBI) Pesticidial crystal protein cry4BA [Bacillus thuringiensis serovar israelensis]: Gene ID: 5759934, updated on 21 Oct. 2014. >gi|161598544|ref|NC_010076.1|:32084-36518 Bacillus thuringiensis serovar israelensis plasmid pBtoxis, complete sequence:

TTAATCTTTGGATTGTTATTATAGCTGTTTTTTTGTTGTATACTCCCGAAAATC
GATTTGAATTTTCTGAATATCGAACAATATATTATTTTGGATGCTTGATAACC
ACTAACGATATGTATGGAAAATTATTTTGAAGTGAAAAAATATGGTCAAATA
AAAATGGAATAATTATATTGGTACAGAAATATGATTGGGATTAGTGAGTCTA
TAATATAGAAAGGAATGTTTTGTTTTTTGTATATAAGTTGAAAAAGATTTCTG
TAAATTGTCCAGAGACTGTATGTGTAGATTGAGTATTGGAACATATCGTTAAT
TTTATATTTTAATATAATGATATGAATTATACAAGGTCTAGATAAGAATTGTT
CATAGGAATCCGTATCAATTTTTTCAAGGAATATGTATTTGCACTTTTGGTCTT
TTTAAATCGTATGAATTCAAAATAGTTTATATCAATCTTTGTTACACCAGAAA
AAGATTGTATCCAATGTGAATATGGGAGGAATAAAT Gene start:
ATGAATTCAGGCTATCCGTTAGCGAATGACTTACAAGGGTCAATGAAAAACA
CGAACTATAAAGATTGGCTAGCCATGTGTGAAAATAACCAACAGTATGGCGT
TAATCCAGCTGCGATTAATTCTTCTTCAGTTAGTACCGCTTTAAAAGTAGCTG
GAGCTATCCTTAAATTTGTAAACCCACCTGCAGGTACTGTCTTAACCGTACTT
AGCGCGGTGCTTCCTATTCTTTGGCCGACTAATACTCCAACGCCTGAAAGAGT
TTGGAATGATTTCATGACCAATACAGGGAATCTTATTGATCAAACTGTAACA
GCTTATGTACGAACAGATGCAAATGCAAAAATGACGGTTGTGAAAGATTATT
TAGATCAATATACAACTAAATTTAACACTTGGAAAAGAGAGCCTAATAACCA
GTCCTATAGAACAGCAGTAATAACTCAATTTAACTTAACCAGTGCCAAACTTC
GAGAGACCGCAGTTTATTTTAGCAACTTAGTAGGTTATGAATTATTGTTATTA
CCAATATACGCACAAGTAGCAAATTTCAATTTACTTTTAATAAGAGATGGCCT
CATAAATGCACAAGAATGGTCTTTAGCACGTAGTGCTGGTGACCAACTATAT
AACACTATGGTGCAGTACACTAAAGAATATATTGCACATAGCATTACATGGT
ATAATAAAGGTTTAGATGTACTTAGAAATAAATCTAATGGACAATGGATTAC
GTTTAATGATTATAAAAGAGAGATGACTATTCAAGTATTAGATATACTCGCTC
TTTTTGCCAGTTATGATCCACGTCGATACCCTGCGGACAAAATAGATAATACG
AAACTATCAAAAACAGAATTTACAAGAGAGATTTATACAGCTTTAGTAGAAT
CTCCTTCTAGTAAATCTATAGCAGCACTGGAGGCAGCACTTACACGAGATGTT
CATTTATTCACTTGGCTAAAGAGAGTAGATTTCTGGACCAATACTATATATCA
AGATTTAAGATTTTTATCTGCCAATAAAATTGGGTTTTCATATACAAATTCTT
CTGCAATGCAAGAAAGTGGAATTTATGGAAGTTCTGGTTTTGGTTCAAATCTT
ACTCATCAAATTCAACTTAATTCTAATGTTTATAAAACTTCTATCACAGATAC
TAGCTCCCCCTCTAATCGAGTTACAAAAATGGATTTCTACAAAATTGATGGTA
CTCTTGCCTCTTATAATTCAAATATAACACCAACTCCTGAAGGTTTAAGGACC
ACATTTTTTGGATTTTCAACAAATGAGAACACACCTAATCAACCAACTGTAAA
TGATTATACGCATATTTTAAGCTATATAAAAACTGATGTTATAGATTATAACA
GTAACAGGGTTTCATTTGCTTGGACACATAAGATTGTTGACCCTAATAATCAA
ATATACACAGATGCTATCACACAAGTTCCGGCCGTAAAATCTAACTTCTTGAA
TGCAACAGCTAAAGTAATCAAGGGACCTGGTCATACAGGGGGGGATCTAGTT
GCTCTTACAAGCAATGGTACTCTATCAGGCAGAATGGAGATTCAATGTAAAA
CAAGTATTTTTAATGATCCTACAAGAAGTTACGGATTACGCATACGTTATGCT
GCAAATAGTCCAATTGTATTGAATGTATCATATGTATTACAAGGAGTTTCTAG
AGGAACAACGATTAGTACAGAATCTACGTTTTCAAGACCTAATAATATAATA
CCTACAGATTTAAAATATGAAGAGTTTAGATACAAAGATCCTTTTGATGCAAT
TGTACCGATGAGATTATCTTCTAATCAACTGATAACTATAGCTATTCAACCAT
TAAACATGACTTCAAATAATCAAGTGATTATTGACAGAATCGAAATTATTCC
AATCACTCAATCTGTATTAGATGAGACAGAGAACCAAAATTTAGAATCAGAA
CGAGAAGTTGTGAATGCACTGTTTACAAATGACGCGAAAGATGCATTAAACA
TTGGAACGACAGATTATGACATAGATCAAGCCGCAAATCTTGTGGAATGTAT
TTCTGAAGAATTATATCCAAAAGAAAAAATGCTGTTATTAGATGAAGTTAAA
AATGCGAAACAACTTAGTCAATCTCGAAATGTACTTCAAAACGGGGATTTTG
AATCGGCTACGCTTGGTTGGACAACAAGTGATAATATCACAATTCAAGAAGA
TGATCCTATTTTTAAAGGGCATTACCTTCATATGTCTGGGGCGAGAGACATTG
ATGGTACGATATTTCCGACCTATATATTCCAAAAAATTGATGAATCAAAATTA
AAACCGTATACACGTTACCTAGTAAGGGGATTTGTAGGAAGTAGTAAAGATG
TAGAACTAGTGGTTTCACGCTATGGGGAAGAAATTGATGCCATCATGAATGT
TCCAGCTGATTTAAACTATCTGTATCCTTCTACCTTTGATTGTGAAGGGTCTA
ATCGTTGTGAGACGTCCGCTGTGCCGGCTAACATTGGGAACACTTCTGATATG
TTGTATTCATGCCAATATGATACAGGGAAAAAGCATGTCGTATGTCAGGATT
CCCATCAATTTAGTTTCACTATTGATACAGGGGCATTAGATACAAATGAAAAT
ATAGGGGTTTGGGTCATGTTTAAAATATCTTCTCCAGATGGATACGCATCATT
AGATAATTTAGAAGTAATTGAAGAAGGGCCAATAGATGGGGAAGCACTGTC
ACGCGTGAAACACATGGAGAAGAAATGGAACGATCAAATGGAAGCAAAACG
TTCGGAAACACAACAAGCATATGATGTAGCGAAACAAGCCATTGATGCTTTA
TTCACAAATGTACAAGATGAGGCTTTACAGTTTGATACGACACTCGCTCAAAT
TCAGTACGCTGAGTATTTGGTACAATCGATTCCATATGTGTACAATGATTGGT
TGTCAGATGTTCCAGGTATGAATTATGATATCTATGTAGAGTTGGATGCACGA
GTGGCACAAGCGCGTTATTTGTATGATACAAGAAATATTATTAAAAATGGTG
ATTTTACACAAGGGGTAATGGGGTGGCATGTAACTGGAAATGCAGACGTACA
ACAAATAGATGGTGTTTCTGTATTGGTTCTATCTAATTGGAGTGCTGGCGTAT
CTCAAAATGTCCATCTCCAACATAATCATGGGTATGTCTTACGTGTTATTGCC
AAAAAAGAAGGACCTGGAAATGGGTATGTCACGCTTATGGATTGTGAGGAG
AATCAAGAAAAATTGACGTTTACGTCTTGTGAAGAAGGATATATTACGAAGA
CAGTAGATGTATTCCCAGATACAGATCGTGTACGAATTGAGATAGGCGAAAC
CGAAGGTTCGTTTTATATCGAAAGCATTGAATTAATTTGCATGAACGAGTGAT
TAATAAAAAATAACTAAAGCTTTAAAAACCATGGAGAAAGTTTTCTCCATGG
TTTTTAATTTCTGCATTTATTAATTCTGGTACAAAAAATATATAGAAAACATA
AAAAATAGATATCTAGAGGACATAAAATTTATACAAATATCAATTTCATTAG
TATAGAACGTTTATCCAATAATAATTATCACCATTTAAACTATCCAAACTACA
ATTGTGATCCAAGTTATGATGATTAATAGATGAACTGATTTAGGATTTGAAAA
TAGATTTTCATCAAACAAAATGAGGGAAAGAATATGCAGGGAAATCAACAAT
CGATTTCAAATCTTAATACTTCGGGATTATTTCGGGGTACAGCCGAGATACGT
GTAAGAATAGACCGTATCCTTACCTTTATTTCTATATTGGATTTATTGAGCTTA
TAAATATTGTTTCTGTGGTTTCGTCTATTTTTATTAAGTAAATTGTCTATTATG
GGTTAACCCTAATCATTCATTAGTTACTGAAAAC

SEQ ID NO:03. A condon modified Cry4Ba: 3408 nucleotides. 78% identical over 86% of the sequence.

ATGCAATCAGGTTACCCATTAGCTAACGACTTACAAGGTTCAATGAAAAACA
CTAACTACAAAGACTGGTTAGCTATGTGTGAAAACAACCAACAATACGGTGT
AAACCCAGCTGCTATTAACTCATCATCAGTATCAACTGCTTTAAAAGTAGCTG
GTGCTATTTTAAAATTCGTAAACCCACCAGCTGGTACTGTATTAACTGTATTA
TCAGCTGTATTACCAATTTTATGGCCAACTAACACTCCAACTCCAGAACGTGT
ATGGAACGACTTCATGACTAACACTGGTAACTTAATTGACCAAACTGTAACT
GCTTACGTACGTACTGACGCTAACGCTAAAATGACTGTAGTAAAAGACTACT
TAGACCAATACACTACTAAATTCAACACTTGGAAACGTGAACCAAACAACCA
ATCATACCGTACTGCTGTAATTACTCAATTCAACTTAACTTCAGCTAAATTAC
GTGAAACTGCTGTATACTTCTCAAACTTAGTAGGTTACGAATTATTATTATTA
CCAATTTACGCTCAAGTAGCTAACTTCAACTTATTATTAATTCGTGACGGTTT
AATTAACGCTCAAGAATGGTCATTAGCTCGTTCAGCTGGTGACCAATTATACA
ACACTATGGTACAATACACTAAAGAATACATTGCTCACTCAATTACTTGGTAC
AACAAAGGTTTAGACGTATTACGTAACAAATCAAACGGTCAATGGATTACTT
TCAACGACTACAAACGTGAAATGACTATTCAAGTATTAGACATTTTAGCTTTA
TTCGCTTCATACGACCCACGTCGTTACCCAGCTGACAAAATTGACAACACTA
AATTATCAAAAACTGAATTCACTCGTGAAATTTACACTGCTTTAGTAGAATCA
CCATCATCAAAATCAATTGCTGCTTTAGAAGCTGCTTTAACTCGTGACGTACA
CTTATTCACTTGGTTAAAACGTGTAGACTTCTGGACTAACACTATTTACCAAG
ACTTACGTTTCTTATCAGCTAACAAAATTGGTTTCTCATACACTAACTCATCA
GCTATGCAAGAATCAGGTATTTACGGTTCATCAGGTTTCGGTTCAAACTTAAC
TCACCAAATTCAATTAAACTCAAACGTATACAAAACTTCAATTACTGACACTT
CATCACCATCAAACCGTGTAACTAAAATGGACTTCTACAAAATTGACGGTAC
TTTAGCTTCATACAACTCAAACATTACTCCAACTCCAGAAGGTTTACGTACTA
CTTTCTTCGGTTTCTCAACTAACGAAAACACTCCAAACCAACCAACTGTAAAC
GACTACACTCACATTTTATCATACATTAAAACTGACGTAATTGACTACAACTC
AAACCGTGTATCATTCGCTTGGACTCACAAAATTGTAGACCCAAACAACCAA
ATTTACACTGACGCTATTACTCAAGTACCAGCTGTAAAATCAAACTTCTTAAA
CGCTACTGCTAAAGTAATTAAAGGTCCAGGTCACACTGGTGGTGACTTAGTA
GCTTTAACTTCAAACGGTACTTTATCAGGTCGTATGGAAATTCAATGTAAAAC
TTCAATTTTCAACGACCCAACTCGTTCATACGGTTTACGTATTCGTTACGCTG
CTAACTCACCAATTGTATTAAACGTATCATACGTATTACAAGGTGTATCACGT
GGTACTACTATTTCAACTGAATCAACTTTCTCACGTCCAAACAACATTATTCC
AACTGACTTAAAATACGAAGAATTCCGTTACAAAGACCCATTCGACGCTATT
GTACCAATGCGTTTATCATCAAACCAATTAATTACTATTGCTATTCAACCATT
AAACATGACTTCAAACAACCAAGTAATTATTGACCGTATTGAAATTATTCCA
ATTACTCAATCAGTATTAGACGAAACTGAAAACCAAAACTTAGAATCAGAAC
GTGAAGTAGTAAACGCTTTATTCACTAACGACGCTAAAGACGCTTTAAACAT
TGGTACTACTGACTACGACATTGACCAAGCTGCTAACTTAGTAGAATGTATTT
CAGAAGAATTATACCCAAAAGAAAAAATGTTATTATTAGACGAAGTAAAAA
ACGCTAAACAATTATCACAATCACGTAACGTATTACAAAACGGTGACTTCGA
ATCAGCTACTTTAGGTTGGACTACTTCAGACAACATTACTATTCAAGAAGACG
ACCCAATTTTCAAAGGTCACTACTTACACATGTCAGGTGCTCGTGACATTGAC
GGTACTATTTTCCCAACTTACATTTTCCAAAAAATTGACGAATCAAAATTAAA
ACCATACACTCGTTACTTAGTACGTGGTTTCGTAGGTTCATCAAAAGACGTAG
AATTAGTAGTATCACGTTACGGTGAAGAAATTGACGCTATTATGAACGTACC
AGCTGACTTAAACTACTTATACCCATCAACTTTCGACTGTGAAGGTTCAAACC
GTTGTGAAACTTCAGCTGTACCAGCTAACATTGGTAACACTTCAGACATGTTA
TACTCATGTCAATACGACACTGGTAAAAAACACGTAGTATGTCAAGACTCAC
ACCAATTCTCATTCACTATTGACACTGGTGCTTTAGACACTAACGAAAACATT
GGTGTATGGGTAATGTTCAAAATTTCATCACCAGACGGTTACGCTTCATTAGA
CAACTTAGAAGTAATTGAAGAAGGTCCAATTGACGGTGAAGCTTTATCACGT
GTAAAACACATGGAAAAAAAATGGAACGACCAAATGGAAGCTAAACGTTCA
GAAACTCAACAAGCTTACGACGTAGCTAAACAAGCTATTGACGCTTTATTCA
CTAACGTACAAGACGAAGCTTTACAATTCGACACTACTTTAGCTCAAATTCAA
TACGCTGAATACTTAGTACAATCAATTCCATACGTATACAACGACTGGTTATC
AGACGTACCAGGTATGAACTACGACATTTACGTAGAATTAGACGCTCGTGTA
GCTCAAGCTCGTTACTTATACGACACTCGTAACATTATTAAAAACGGTGACTT
CACTCAAGGTGTAATGGGTTGGCACGTAACTGGTAACGCTGACGTACAACAA
ATTGACGGTGTATCAGTATTAGTATTATCAAACTGGTCAGCTGGTGTATCACA
AAACGTACACTTACAACACAACCACGGTTACGTATTACGTGTAATTGCTAAA
AAAGAAGGTCCAGGTAACGGTTACGTAACTTTAATGGACTGTGAAGAAAACC
AAGAAAAATTAACTTTCACTTCATGTGAAGAAGGTTACATTACTAAAACTGT
AGACGTATTCCCAGACACTGACCGTGTACGTATTGAAATTGGTGAAACTGAA
GGTTCATTCTACATTGAATCAATTGAATTAATTTGTATGAACGAA

The following references are herein incorporated by reference in their entirety:
1. Angsuthanasombat C, Crickmore N and Ellar D J (1992) Comparison of Bacillus thuringiensis subsp. israelensis CryIVA and CRYIVB cloned toxins reveals synergism in vivo. FEMS Microbiol Lett 94:63-68
2. Anthonisen I L, Kasai S, Kato K, Salvador M L and Klein U (2002) Structural and functional characterization of a transcription-enhancing sequence element in the rbcL gene of the Chlamydomonas chloroplast genome. Curr Genet 41:349-356
3. Barnes D, Franklin S, Schultz J, Henry R Brown E, Coragliotti A and Mayfield S P (2005) Contribution of 5′- and 3′-untranslated regions of plastid mRNAs to the expression of Chlamydomonas reinhardtii chloroplast genes. Mol Gen Genomics 274:625-636
4. Becker N (1997) Microbial Control of Mosquitoes: Management of the Upper Rhine Mosquito Population as a Model Programme. Parasitol. Today 11:399-402
5. Boonserm P, Davis P, Ellar D J and Li J (2005) Crystal structure of the mosquito-larvicidal toxin Cry4Ba and its biological implications. J Mol Biol 348:363-382
6. Boonserm P, Mo M, Angsuthanasombat C and Lescar J (2006) Structure of the functional form of the mosquito larvicidal Cry4Aa toxin from Bacillus thuringiensis at a 2.8-Angstrom resolution. J Bacteriology 188:3391-3401
7. Boussiba S, Wu X-Q, Ben-Dov E, Zarka A and Zaritsky A (2000) Nitrogen-fixing cyanobacteria as gene delivery systems for expressing mosquitocidal toxins of Bacillus thuringiensis ssp. israelensis. J Appl Phycol 12:46-467
8. Bravo A, Likitvivatanavong S, Gill S S and Soberon M (2011) Bacillus thuringiensis: a story of a successful bioinsecticide. Insect Biochem Mol Biol 41:423-431
9. Brizzard B (2008) Epitope tagging. Biotechniques 44:693-695
10. Cohen S, Albeck S, Ben-Dove E, Cahan R, Firer M, Zaritsky A and Dym O (2011) Cyt1Aa toxin: crystal structure reveals implications for its membrane-perforating function. J Mol Biol 413:804-814
11. Coragliotti A T, Beligni M V, Franklin S E and Mayfield S P (2011) Molecular Factors Affecting the Accumulation of Recombinant Proteins in the Chlamydomonas reinhardtii Chloroplast. Mol Biotechnol 48:60-75
12. Dauvillee D, Delhaye S, Gruyer S, Slomianny C, Moretz S E, d'Hulst C, Long C A, Ball S G and Tomavo S (2010) Engineering the chloroplast targeted malarial vaccine antigens in Chlamydomonas Starch Granules 5:e15424
13. Dulmage H T, Yousten A A, Singer S and Lacey L A (1990) Guidelines for the production of Bacillus thuringiensis H-14 and Bacillus sphaericus. World Health Organization, Geneva
14. Fargo D C, Zhang M, Gillham N W and Boynton J E (1998) Shine-Dalgarno-like sequences are not required for translation of chloroplast mRNAs in Chlamydomonas reinhardtii chloroplasts or in Escherichia coli. Mol Gen Genet 257:271-282
15. Fischer Nicolas, Stampacchia Otello, Redding Kevin and Rochaix Jean-David (1996) Selectable marker recycling in the chloroplast. Mol Gen Genet 251: 373-380
16. Goldschmidt-Clermont M (1998) Chloroplast transformation and reverse genetics. In Molecular Biology of Chloroplasts and Mitochondria in Chlamydomonas. (Rochaix J-D, Goldschmidt-Clermont M and Merchant S, eds). Academic Press, San Diego
17. Herrin, D L, Michaels A S, Paul A S (1986) Regulation of genes encoding the large subunit of ribulose-1,5-bisphosphate carboxylase and the photosystem II polypeptides D-1 and D-2 during the cell cycle of Chlamydomonas reinhardtii. J Cell Biol 103:1837-1845.
18. Hate H and Whiteley H R (1989) Insecticidal crystal proteins of Bacillus thuringiensis. Microbiol Rev 53: 242-255
19. Ji Q, Vincken J-P, Suurs L C J M and Visser R G F (2003) Microbial starch-binding domains as a tool for targeting proteins to granules during starch biosynthesis. Plant Mol Biol 51:789-801
20. Kaufman M G, Wanj a E, Maknojia S, Bayoh M N, Vulule J M and Walker E D (2006) Importance of algal biomass to growth and development of Anopheles gambiae larvae. J Med Entomol 43:669-676
21. Khasdan V, Ben-Dov E, Manasherob R, Boussiba S and Zaritsky A (2001) Toxicity and synergism in transgenic Escherichia coli expressing four genes from Bacillus thuringiensis subsp. Israelensis. Environmental Microbiology 3:798-806
22. Laird, M (1988) The Natural History of Larval Mosquito Habitats. Academic Press, London
23. Lister Diane L, Bateman Joseph M, Purton Saul and Howe Christopher J (2003) DNA transfer from chloroplast to nucleus is much rarer in Chlamydomonas than in tobacco. Gene 316:33-38
24. Liu Y-T, Sui M-J, Dar-Der J I, Wu I-H, Chou C-C and Chen C-C (1993) Protection from ultraviolet irradiation by melanin of mosquitocidal activity of Bacillus thuringiensis var. israelensis J Invertebr Pathol 62:131-136
25. Marten G G (1986) Mosquito control by plankton management: the potential of indigestible green algae. J Trop Med Hyg 89:213-222
26. McBride K, Svab Z, Schaaf D J, Hogan P S, Stalker D M and Maliga P (1995) Amplification of a chimeric Bacillus gene in chloroplasts leads to an extraordinarily high level of an insecticidal protein in tobacco. Biotechnology 13:362-365
27. Michelet Laure, Lefebvre-Legendre Linnka, Burr Sarah E, Rochaix Jean-David and Goldschmidt-Clermont Michel (2010) Enhanced chloroplast transgene expression in a nuclear mutant of Chlamydomonas. Plant Biotechnol J 9:564-574
28. Minko I, Holloway S P, Nikaido S, Odom O W, Carter M, Johnson C H and Herrin D L (1999) Renilla luciferase as a vital reporter for chloroplast gene expression in Chlamydomonas. Mol Gen Genet 262:421-425
29. Mulla, M S (1985) Field evaluation and efficacy of bacterial agents and their formulations against mosquito larvae. In Integrated Mosquito Control Methodologies, Vol. 2. Academic Press, London, pp 227-250
30. Muto M, Henry R E and Mayfield S P (2009) Accumulation and processing of a recombinant protein designed as a cleavable fusion to the endogenous Rubisco LSU protein in Chlamydomonas chloroplast. BMC Biotechnology 9:26
31. Nickelsen J, Fleischmann M, Boudreau E, Rahire M and Rochaix J-D (1999) Identification of cis-acting RNA leader elements required for chloroplast psbD gene expression in Chlamydomonas. Plant Cell 11:957-970
32. Peferoen M (1997) Insect control with transgenic plants expressing Bacillus thuringiensis crystal proteins. In Advances in Insect Control. The Role of Transgenic Plants. Taylor and Francis, London, pp 21-48
33. Poncet S, Delécleuse A, Klier A and Rapoport G (1995) Evaluation of synergistic interactions among the CryIVA, CryIVB, and CryIVD toxic components of B. thuringiensis subsp. israelensis. J Invertebr Pathol 66:131-135
34. Proschold T, Harris E and Coleman A W (2005) Portrait of a Species: Chlamydomonas reinharditii. Genetics 170:1601-1610
35. Rasala B A, Muto M, Lee P A, Jager M, Cardoso R M F, Behnke C A, Kirk P, Hokanson C A, Crea R, Mendez M and Mayfield S P (2010) Production of therapeutic proteins in algae, analysis of expression of seven human proteins in the chloroplast of Chlamydomonas reinhardtii. Plant Biotechnology J 8:719-733
36. Rasala B A, Muto M, Sullivan J and Mayfield S P (2011) Improved heterologous protein expression in the chloroplast of Chlamydomonas reinhardtii through promoter and 5′ untranslated region optimization. Plant Biotech. Journal 9:674-683
37. Rivier C, Goldschmidt-Clermont M and Rochaix J-D (2001) Identification of an RNA-protein complex involved in chloroplast group II intron trans-splicing. EMBO J 20:1765-1773
38. SAS Institute (1985) The Probit procedure. In SAS User=s Guide. Cary, N.C. pp 639-645
39. Schnepf E, Crickmore N, Van Rie J, Lereclus D, Baum J, Feitelson J, Zeigler D R and Dean D H (1998) Bacillus thuringiensis and its pesticidal crystal proteins. Microbiol Mol Biol Rev 62:775-806
40. Smith C N (1971) Insect colonization and mass production. Academic Press, New York.
41. Surzycki R, Cournac L, Peltier G and Rochaix J-D (2007) Potential for hydrogen production with inducible chloroplast gene expression in Chlamydomonas. PNAS 104:17548-17553
42. Uniacke J and Zerges W (2009) Chloroplast protein targeting involves localized translation in Chlamydomonas. PNAS 106:1439-1444
43. Van Frankenhuyzen K (2009) Insecticidal activity of Bacillus thuringiensis crystal proteins. J Invertebr Pathol 101:1-16
44. Wu X Q, Vennison Sj, Huirong L, Ben-Dov E, Zaritsky A and Boussiba S (1997)
Mosquito larvacidal activity of transgenic Anabaena strain PCC 7120 expressing combinations of genes from Bacillus thuringiensis subsp. israelensis. Appl Environ Microbiol 63:4971-4975
45. Xu Y, Nagal M, Bagdasarian M, Smith T W and Walker E D (2001) Expression of the p20 gene from Bacillus thuringiensis H-14 increases cry11A toxin production and enhances mosquito-larvicidal activity in recombinant gram-negative bacteria. Appl Environ Microbiol 67:3010-3015
46. Zaritsky A, Ben-Dov E, Borovsky D, Boussiba S, Einav M, Gindin G, Horowitz A R, Kolot M, Melnikov O, Mendel Z and Yagil E (2010) Transgenic organisms expressing genes from Bacillus thuringiensis to combat insect pests. Bioengineered Bugs 1:341-344
47. Zicker A A, Kadakia C S and Herrin D L (2007) Distinct roles for the 5′ and 3′ untranslated regions in the degradation and accumulation of chloroplast tufA mRNA: Identification of an early intermediate in the in vivo degradation pathway. Plant Mol Biol 63: 689-702.

The following references are herein incorporated by reference in their entirety:
1. Abdullah M A F and Dean D H (2004) Enhancement of Cry19Aa Mosquitocidal Activity against Aedes aegypti by Mutations in the Putative Loop Regions of Domain II. Appl Environ Microbiol 70:3769-3771.
2. Angsuthanasombat C, Crickmore N and Ellar D J (1992) Comparison of Bacillus thuringiensis subsp. israelensis CryIVA and CRYIVB cloned toxins reveals synergism in vivo. FEMS Microbiol Lett 94:63-68.
3. Anthonisen I L, Kasai S, Kato K, Salvador M L and Klein U (2002) Structural and functional characterization of a transcription-enhancing sequence element in the rbcL gene of the Chlamydomonas chloroplast genome. Curr Genet 41:349-356.
4. de Barjac H and Sutherland D, Editors (1990) Bacterial Control of Mosquitoes and Black Flies. Rutgers University Press, New Brunswick, N.J.
5. Barnes D, Franklin S, Schultz J, Brown E, Coragliotti A and Mayfield S P (2005) Contribution of 5′- and 3′-untranslated regions of plastid mRNAs to the expression of Chlamydomonas reinhardtii chloroplast genes. Mol Gen Genomics 274:625-636.
6. Boonserm P, Davis P, Ellar D J and Li J (2005) Crystal structure of the mosquito-larvicidal toxin Cry4Ba and its biological implications. J Mol Biol 348:363-382.
7. Boonserm P, Mo M, Angsuthanasombat C and Lescar J (2006) Structure of the functional form of the mosquito larvicidal Cry4Aa toxin from Bacillus thuringiensis at a 2.8-Angstrom resolution. J Bacteriol 188:3391-3401.
8. Borovsky D, Khasdan V, Nauwelaers S, Theunis C, BertieOr L, Ben-Dov E, and Zaritsky A (2010) Synergy between Aedes aegypti trypsin modulating oostatic factor and δ-Endotoxins. Open Toxinol J 3:141-150.
9. Boussiba S, Wu X-Q, Ben-Dov E, Zarka A and Zaritsky A (2000) Nitrogen-fixing cyanobacteria as gene delivery systems for expressing mosquitocidal toxins of Bacillus thuringiensis ssp. israelensis. J Appl Phycol 12:46-467.
10. Bravo A, Likitvivatanavong S, Gill S S and Soberon M (2011) Bacillus thuringiensis: a story of a successful bioinsecticide. Insect Biochem Mol Biol 41:423-431.
11. Brizzard B (2008) Epitope tagging. Biotechniques 44:693-695.
12. CDC. West Nile Virus and other arboviral diseases—United States, 2012. MMWR 2013; 62; 513-517.
13. Chen S, Kaufman M G, Korir M L and Walker E D (2014) Ingestibility, digestibility, and engineered biological control potential of Flavobacterium hibernum isolated from larval mosquito habitats. Appl Environ Microbiol 80:1150-1158.
14. Cohen S, Albeck S, Ben-Dove E, Cahan R, Firer M, Zaritsky A and Dym O (2011) Cyt1Aa toxin: crystal structure reveals implications for its membrane-perforating function. J Mol Biol 413:804-814.
15. Coragliotti A T, Beligni M V, Franklin S E and Mayfield S P (2011) Molecular factors affecting the accumulation of recombinant proteins in the Chlamydomonas reinhardtii chloroplast. Mol Biotechnol 48:60-75.
16. Dauvillee D, Delhaye S, Gruyer S, Slomianny C, Moretz S E, d'Hulst C, Long C A, Ball S G and Tomavo S (2010) Engineering the chloroplast targeted malarial vaccine antigens in Chlamydomonas starch granules. PLOS 5:e15424.
17. Fargo D C, Zhang M, Gillham N W and Boynton J E (1998) Shine-Dalgarno-like sequences are not required for translation of chloroplast mRNAs in Chlamydomonas reinhardtii chloroplasts or in Escherichia coli. Mol Gen Genet 257:271-282.
18. Glare T R and O'Callaghan M (2000) Bacillus thuringiensis: Biology, Ecology and Safety. J Wiley & Sons, West Sussex U K.
19. Goldschmidt-Clermont M (1998) Chloroplast transformation and reverse genetics. In Molecular Biology of Chloroplasts and Mitochondria in Chlamydomonas. (Rochaix J-D, Goldschmidt-Clermont M and Merchant S, eds). Academic Press, San Diego.
20. Hua G, Zhang R, Abdullah M A F and Adang M J (2008) Anopheles gambiae Cadherin AgCad1 binds the Cry4Ba toxin of Bacillus thuringiensis israelensis and a fragment of AgCad1 synergizes toxicity. Biochemistry 47:5101-5110.
21. Herrin, D L, Michaels A S, Paul A L (1986) Regulation of genes encoding the large subunit of ribulose-1,5-bisphosphate carboxylase and the photosystem II polypeptides D-1 and D-2 during the cell cycle of Chlamydomonas reinhardtii. J Cell Biol 103:1837-1845.
22. Ji Q, Vincken J-P, Suurs L C J M and Visser R G F (2003) Microbial starch-binding domains as a tool for targeting proteins to granules during starch biosynthesis. Plant Mol Biol 51:789-801.
23. Kaufman M G, Wanja E, Maknojia S, Bayoh M N, Vulule J M and Walker E D (2006) Importance of algal biomass to growth and development of Anopheles gambiae larvae. J Med Entomol 43:669-676.
24. Kamel F (2013) Paths from pesticides to Parkinson's. Science 341:722-723 25. Kang S, Odom O W, Herrin D L (2013) Mosquito control with green algae: Expression of Cry genes from Bacillus thuringiensis israelensis (Bti) in the chloroplast of Chlamydomonas. 3^{rd} International Conference on Chloroplast Genomics and Genetic Engineering, Rutgers University, New Brunswick, N.J., May 12-15.
26. Khasdan V, Ben-Dov E, Manasherob R, Boussiba S and Zaritsky A (2001) Toxicity and synergism in transgenic Escherichia coli expressing four genes from Bacillus thuringiensis subsp. Israelensis. Environ Microbiol 3:798-806.
27. Laird, M (1988) The Natural History of Larval Mosquito Habitats. Academic Press, London.
28. Lister D L, Bateman J M, Purton S and Howe C J (2003) DNA transfer from chloroplast to nucleus is much rarer in Chlamydomonas than in tobacco. Gene 316:33-38.
29. Liu Y-T, Sui M-J, Dar-Der J I, Wu I-H, Chou C-C and Chen C-C (1993) Protection from ultraviolet irradiation by melanin of mosquitocidal activity of Bacillus thuringiensis var. israelensis. J Invertebr Pathol 62:131-136.
30. Mala A O and Irungu L W (2011) Factors influencing differential larval habitat productivity of Anopheles gambiae complex mosquitoes in a western Kenyan village. J Vector Borne Dis 48:52-57.
31. Marten G G (1986) Mosquito control by plankton management: the potential of indigestible green algae. J Trop Med Hyg 89:213-222.
32. Michelet Laure, Lefebvre-Legendre Linnka, Burr Sarah E, Rochaix Jean-David and Goldschmidt-Clermont Michel (2010) Enhanced chloroplast transgene expression in a nuclear mutant of Chlamydomonas. Plant Biotechnol J 9:564-574.
33. Minko I, Holloway S P, Nikaido S, Odom O W, Carter M, Johnson C H and Herrin D L (1999) Renilla luciferase as a vital reporter for chloroplast gene expression in Chlamydomonas. Mol Gen Genet 262:421-425.
34. Muto M, Henry R E and Mayfield S P (2009) Accumulation and processing of a recombinant protein designed as a cleavable fusion to the endogenous Rubisco LSU protein in the Chlamydomonas chloroplast. BMC Biotechnol 9:26.
35. Nickelsen J, Fleischmann M, Boudreau E, Rahire M and Rochaix J-D (1999) Identification of cis-acting RNA leader elements required for chloroplast psbD gene expression in Chlamydomonas. Plant Cell 11:957-970.
36. Poncet S, Delécleuse A, Klier A and Rapoport G (1995) Evaluation of synergistic interactions among the CryIVA, CryIVB, and CryIVD toxic components of B. thuringiensis subsp. israelensis. J Invertebr Pathol 66:131-135.
37. Proschold T, Harris E and Coleman A W (2005) Portrait of a Species: Chlamydomonas reinharditii. Genetics 170:1601-1610.
38. Rasala B A, Muto M, Lee P A, Jager M, Cardoso R M F, Behnke C A, Kirk P, Hokanson C A, Crea R, Mendez M and Mayfield S P (2010) Production of therapeutic proteins in algae, analysis of expression of seven human proteins in the chloroplast of Chlamydomonas reinhardtii. Plant Biotechnol J 8:719-733.
39. Rasala B A, Muto M, Sullivan J and Mayfield S P (2011) Improved heterologous protein expression in the chloroplast of Chlamydomonas reinhardtii through promoter and 5′ untranslated region optimization. Plant Biotechnol J 9:674-683.
40. Sansinenea E, Editor (2012) Bacillus thuringiensis Biotechnology. Springer, Dordrecht, Netherlands.
41. Sazhenskiy V, Zaritsky A and Itsko M (2010) Expression in Escherichia coli of the native cyt1Aa from Bacillus thuringiensis subsp. israelensis. Appl Environ Microbiol 76:3409-3411.
42. Schnepf E, Crickmore N, Van Rie J, Lereclus D, Baum J, Feitelson J, Zeigler D R and Dean D H (1998) Bacillus thuringiensis and its pesticidal crystal proteins. Microbiol Mol Biol Rev 62:775-806.
43. Sirichotpakorn N, Rongnoparut P, Choosang K, and Panbangred W (2001) Coexpression of chitinase and the cry11Aa1 toxin genes in Bacillus thuringiensis serovar israelensis. J Invertebr Pathol 3:160-169.
44. Surzycki R, Cournac L, Peltier G and Rochaix J-D (2007) Potential for hydrogen production with inducible chloroplast gene expression in Chlamydomonas. Proc Natl Acad Sci USA 104:17548-17553.
45. Uniacke J and Zerges W (2009) Chloroplast protein targeting involves localized translation in Chlamydomonas. Proc Natl Acad Sci USA 106:1439-1444.
46. Wirth M C, Yang Y, Walton W E, Frederici B A and Berry C (2007) Mtx toxins synergize Bacillus sphaericus and Cry11Aa against susceptible and insecticide-resistant Culex quinquefasciatus larvae. Appl Environ Microbiol 73:6066-6071.
47. Wu X Q, Vennison Sj, Huirong L, Ben-Dov E, Zaritsky A and Boussiba S (1997) Mosquito larvacidal activity of transgenic Anabaena strain PCC 7120 expressing combinations of genes from Bacillus thuringiensis subsp. israelensis. Appl Environ Microbiol 63:4971-4975.
48. Xu Y, Nagal M, Bagdasarian M, Smith T W and Walker E D (2001) Expression of the p20 gene from Bacillus thuringiensis increases cry11A toxin production and enhances mosquito-larvicidal activity in recombinant gram-negative bacteria. Appl Environ Microbiol 67:3010-3015.
49. Zaritsky A, Ben-Dov E, Borovsky D, Boussiba S, Einav M, Gindin G, Horowitz A R, Kolot M, Melnikov O, Mendel Z and Yagil E (2010) Transgenic organisms expressing genes from Bacillus thuringiensis to combat insect pests. Bioengineered Bugs 1:341-344.
50. Zicker A A, Kadakia C S and Herrin D L (2007) Distinct roles for the 5′ and 3′ untranslated regions in the degradation and accumulation of chloroplast tufA mRNA: Identification of an early intermediate in the in vivo degradation pathway. Plant Mol Biol 63: 689-702.

### EXPERIMENTAL

The following examples serve to illustrate certain embodiments and aspects of the present invention and are not to be construed as limiting the scope thereof. In the experimental disclosures which follow, the following abbreviations apply: N (normal); M (molar); mM (millimolar); microM (micromolar); mol (moles); mmol (millimoles); micro.mol (micromoles); nmol (nanomoles); pmol (picomoles); g (grams); mg (milligrams); microg (micrograms); ng (nanograms); pg (picograms); L and (liters); ml (milliliters); microl (microliters); cm (centimeters); mm (millimeters); microm (micrometers); nm (nanometers); U (units); min (minute); s and sec (second); deg (degree); and ° C. (degrees Centigrade/Celsius).

### Example I

The following describes exemplary materials and methods used during development of the present inventions.

Native Bti proteins (i.e. Cry proteins produced by Bti bacteria) are considered protoxins until they become fully activated toxins in the larval gut. Expression of these protoxin genes in heterologous bacteria showed that the expressed protein also have toxic activity, and that their effect on different hosts is variable and unpredictable. Therefore, to overcome these limitations, the inventors contemplated expresssing Cry genes in an inducible chloroplast gene expression system developed in the Rochaix lab (Surzycki et al. 2007) for expresssing CRY protwins in the chloroplast of living Chlamydomonas.

Exempalry Cry and Cyt Genes, contructs, expression systems, analysis methods and larvicidal bioassys are described herein that were used during the development of the present inventions.

A. Design and Preparation of Synthetic Genes.

Attempts to express copies of native Cry11Aa gene in the C. reinhardtii chloroplast were unsuccessful in that no protein was detected using an anti-Cry11Aa antibody. The inventors' did successfully express a native luciferase gene in the chloroplast, however that protein was small at about 35 kDa (Minko et al., 1999), whereas the Cry proteins are 72 to 145 kDa in size. Because codon bias is one of several limiting factors for expressing foreign genes in the Chlamydomonas chloroplast (Franklin et al., 2002; Mayfield et al., 2003) the inventor's developed codon modified Cry genes.

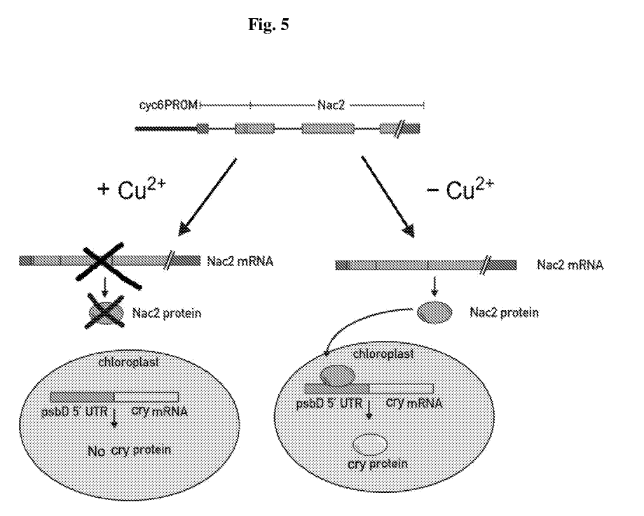

Thus synthetic genes for CRY coding regions were designed using the native amino acid sequences for reverse engineering the encoding DNA of the present inventions having modified codon usage in part, found in the Chlamydomonas chloroplast DNA sequences. For Cry4A, DNA encoding a partial protein of aa 1-700 was synthesized, creating Cry4A 700 protein, Cry4A truncated after amino acid 700. Whereas DNA sequences (i.e. genes) encoding complete Cry4B (135 kDa) and Cry11A (72 kDa) proteins was synthesized. A Flag epitope tag was added to the C-terminus of each protein (by ligating the encoding DNA to the Cry DNA sequence), to enable antibody-based detection and comparisons of expression. See, schematics shown in FIG. 5A.

Thus, synthetic Cry4Aa700, Cry4Ba, and Cry11Aa genes were designed based on the codon usage of 8 highly expressed chloroplast genes from Chlamydomonas, such that the Codon Adaptive Index increased from ˜0.5 to 1 after optimization (FIG. 6). Also, for Cry4Aa, merely the first 700 amino acids were used in order to increase the chances of expression, as larger proteins tend to be less abundant than smaller proteins (Bernaudat et al., 2011). The 8-amino acid Flag tag (Einhauer and Jungbauer, 2001) was added (ligated) to the C-terminus of all three Cry protein genes to make it possible to detect them with a commercial antibody (FIG. 6B). This tag was expected to have little effect on insect toxicity, since the terminal amino acids are cleaved off in the insect gut. Before attempting chloroplast expression, these 3 genes were expressed in E. coli using the inducible pET system (Studier et al., 1990). The expected protein sizes were obtained for each of these 3 genes.

More specific descriptions of codon optimization of Cry4Ba, Cry11Aa, and a truncated version of Cry4Aa containing amino acids (aa) 1-700, Cry4Aa₇₀₀, sequences of the corresponding Bti genes (NCBI Gene Ids as follows: Cry4Aa—5759905, Cry4Ba—5759934, Cry11Aa-5759849). The program Optimizer (Puigbò et al., 2007) was also used. A codon-usage table was developed and used by the inventors after analyzing 8 highly expressed chloroplast genes, which was different from the codon usage table that is based on an entire set of chloroplast-encoded ORFs obtained from the chloroplast genome (see, Kazusa University web site). The protein sequences of the present inventions also contained an 8-aa Flag peptide DYKDDDDK at the C-terminus to enable their detection on western blots by binding to commercial Flag antibodies. After analyzing the predicted RNA structures at the 5′ end of the genes using Mfold, the third codon in the optimized Cry4Ba sequence was changed from AAC to CAA, which changed the aa from asparagine (N) to glutamine (Q). This was done to prevent an unfavorable secondary structure that would have tied up the start codon in a paired region. The 3 genes were synthesized by Integrated DNA Technologies (IDT: 1710 Commercial Park, Coralville, Iowa 52241 USA). This company also confirmed the sequence of the genes and provided them cloned into plasmids.

B. Constructing Cry Plasmids for Inducible Expression.

Construction of the plasmids for inducible Cry gene expression was carried out using E. coli DH5a (Invitrogen) as the host, and were assembled in the low-copy pET-16b plasmid. The codon-adapted Cry genes (from IDT) were excised from the IDT plasmids by digestion with XbaI (on the 3′ side), blunting with the Klenow DNA polymerase, and then digestion with NdeI (on the 5′ side). The Cry4Aa₇₀₀ and Cry11Aa genes were ligated to pET-16b that had been cut with XhoI, blunted with Klenow, and then cut with NdeI. However, the NdeI digestion was incomplete and so the clones turned out to have 9 extra nucleotides (3 amino acids, MLD) at the beginning of the coding sequence that included an intact NdeI site. Thus Cry11Aa of the present inventions included a sequence having MLD in front of the first (ATG) M shown in FIG. 6A and in addition to MLD at the beginning of the novel Cry4Aa₇₀₀. For Cry4Ba subcloning, pET-16B was digested with BamHI instead of XhoI, then blunted and digested with NdeI; so the Cry4Ba clone did not have extra nucleotides at the 5′ end. The new plasmids were pET-4A₇₀₀, pET-4B, and pET-11A.

The 5′ and 3′ expression signals (from psbD and psbA, respectively) were added sequentially to the Cry genes as PCR products made with the high-fidelity Phusion DNA polymerase (from New England Biolabs) according to the manufacturer's instruction. The primers used for the PCR reactions are listed in Table 1, and the thermocycling program was as follows: 94° C. for 3 minutes; 33 cycles of 52° C. (1 minute), 72° C. (3.5 minutes), and 94° C. (30 seconds); 52° C. (1 minute); and then 72° C. (5 minutes). The PCR products were analyzed on 1% agarose gels before restriction digestion and cloning.

The 5′ expression signals for the psbD gene, including the promoter and 5′-UTR, were amplified from plasmid p108-14 (Surzycki et al., 2007) which has the EcoRI R3 fragment of the chloroplast genome of C. reinhardtii. p108-14 was obtained from Jean-David Rochaix (U of Geneva). PCR products were analyzed on 1% agarose gels before restriction digestion and cloning. The forward primer (847 in Table 1) contained overlapping NcoI and BamHI sites; the NcoI site was used to attach it to the coding regions (as a NcoI-NdeI fragment) and the BamHI site was used later to excise the whole gene for subcloning into the chloroplast transformation plasmid. The reverse primer (850 in Table 1) contained an NdeI site—for attaching it to the coding region—but also altered the possible Shine-Dalgano sequence at nucleotides −13 to −9 from GGAG to AAAG (Nickelsen et al. 1999); this mutation was introduced to block translation in E. coli. The altered 5′ region was called psbDₘ, and the resulting PCR product was double-digested with NcoI and NdeI and cloned into the NcoI+NdeI-digested pET-Cry plasmids (above); the new plasmids were called pET-5D4A₇₀₀, pET-5D4B, and pET-5D11A.

TABLE 1
Exemplary oligonucleotide sequences (PCR F (5′-3′)
and R (3′-5′) primers) and short sequences used
during the development of the present inventions.

| ID | | | SEQ |
|---|---|---|---|
| no. | Name^{a} | Sequence^{b} | ID NO: |
| 795 | Cry4A F | GTCAACAAAACCAACAATACG | 4 |
| 796 | Cry4A R | TTAGTGTAGTCAGTACCTGAG | 5 |
| 797 | Cry4B F | AACGACTTACAAGGTTCAATG | 6 |
| 798 | Cry4B R | TGTCTGGGAATACGTCTACAG | 7 |
| 799 | Cry11A F | GGAAGACTCATCATTAGACAC | 8 |
| 800 | Cry11A R | AGTAGCAGTGTTGAAACCAGT | 9 |
| 802 | T7 prom | GAAATTAATACGACTCACTATAGG | 10 |
| | pET | | |
| 803 | T7 ter | GCTAGTTATTGCTCAGCGG | 11 |
| | pET | | |
| 847 | psbD 5′ F | gctcccatggatccTCATAATAATAAAACC | 12 |
| | | TTTATTCAT NcoI BamHI | |
| 850 | psbD 5′ R | ccggcatatgGTGTATCTTTAAAATAAAAA | 13 |
| | | AACAACTCATCGTTACG | |
| | | NdeI | |
| 860 | psbA 3′ F | cggggctgAGCTCAAACAACTAATTTTTTT | 14 |
| | | TTAAAC BlpI | |
| 861 | psbA 3′ R | cagtgctcagcggaTCCTGCCAACTGCCTA | 15 |
| | | TGGTAGC BlpI BamHI | |
| 864 | Integration | TGGAATTGGATATGGACTAG | 16 |
| | site F | | |
| 865 | Integration | GGTACTTGCATTTCATAAGT | 17 |
| | site R | | |

^{a}F, forward; R, reverse
^{b}Upper case letters, Cry or chloroplast gene nucleotides; underlined letters, nucleotides used to generate restriction sites; lower case letters (not underlined), additional nucleotides added to increase digestion efficiency. Bold and gray-shaded TT nts in psbD5′R are substitutions of the normal CC nts, in order to eliminate the Shine-Dalgarno-like sequence.

To add the psbA 3′ region, it was amplified from plasmid P-322 (Newman et al., 1992; Chlamydomonas Culture Center) with primers 860 and 861 (Table 1). Both primers contained a BlpI site for subcloning the product downstream of the Cry coding regions, and the reverse primer (861) also contained a BamHI site for cloning the whole construct into a chloroplast transformation plasmid (see below). The PCR product was cut with BlpI and cloned into Bpu1102I-cut pET-5D4A₇₀₀, pET-5D4B, and pET-5D11A, where it attaches in one direction. This added ˜50 nucleotides of the vector to the end of the coding region, preceding the 3′ UTR from psbA. The new plasmids were called pET-5D4A₇₀₀3 A, pET-5D4B3A, and pET-5D11A3A. The psbaₘ-Cry-psbA constructs were confirmed by sequencing.

To create the chloroplast transformation plasmids, the Cry gene constructs were excised with BamHI and cloned into the BamHI site of p322.1, which corresponds to the intergenic region between the psbA and the 23 S rRNA genes (in the inverted repeat of CpDNA). The final plasmids were called pCry4A₇₀₀, pCry4B, and pCry11A.

C. Biolistic Bombardment for Transformation.

The following is an exemplary chloroplast transformation method for the Ind41_18 strain. For transformation, the Ind41_18 strain was grown in liquid TAP medium under a light flux of ca. 40 μE m⁻² sec⁻¹ at 23° C. The cultures were mixed continuously on an orbital shaker (125 rpm) until they reached the late log/early stationary phase (2×10⁶-4×10⁶ cells/mL). The cells were collected by centrifugation, and resuspended in fresh TAP to a concentration of ˜1×10⁸ cells/mL; cell number was approximated from chlorophyll content (Arnon, 1949; Harris, 1989). 0.4 mL of the cells (˜4×10⁷) was mixed with 0.4 mL of molten 0.25% agar in TAP minimal medium. 0.8 mL of the mixture was pipetted onto the center of a TAP-agar plate containing 100 μg/mL of ampicillin, and allowed to air dry.

Biolistic transformation of the Ind41_18 chloroplast with the Cry plasmids was performed as described by Odom et al. (2001) using co-transformation with plasmid pB4CC110. pB4CC110 harbors a 7-kb BamHI fragment of CpDNA that contains the spectinomycin-resistance marker, spr-u-1-6-2, in the 16S rrn gene (Harris, 1989; Newman et al., 1990). 5 μg of pB4CC110 and an equal amount of one of the Cry plasmids were co-precipitated onto 3 mg of tungsten particles (M17, Bio-Rad), and about 600 ng of plasmid DNA was shot at each plate of cells embedded in a layer of soft agar (Boynton and Gillham, 1993). The bombarded plates were incubated overnight in dim light (ca. 2 μE m⁻² sec⁻¹), then the cell layer of each was scraped off and split onto two TAP-agar plates containing 100-μg/mL spectinomycin. The selection plates were incubated under bright light (ca. 40 μE m⁻² sec⁻¹) at 23° C., and spectinomycin-resistant colonies appeared in 2-4 weeks. The colonies were re-streaked and grown several times on TAP-agar containing 300 μg/mL spectinomycin until they reached homoplasmicity as judged by PCR.D.

D. Expression Systems.

The following describe several expression systems used durng the development of the present inventions.

1. Mayfield Lab.

pD1-KanR (obtained from S Mayfield, University of California, San Diego) is a Chlamydomonas chloroplast transformation plasmid that can give one of the highest levels of transgene expression. The foreign gene is expressed using 5′ and 3′ signals from psbA and the transgene actually replaces the endogenous psbA gene during transformation (Rasala et al., 2010). The codon-adapted Cry4A₇₀₀, Cry4Ba, and Cry11Aa genes were excised from their original plasmids by NdeI+XbaI digestion and ligated into NdeI+XbaI-digested pD1-KanR to give plasmids pD1-4A, pD1-4B, and pD1-11A, respectively. These plasmids were transformed into the chloroplast of a wild-type strain (2137 mt+) using biolistic bombardment (Chloroplast transformation of the Ind41_18 strain), and transformants were selected on kanamycin (100 μg/mL) plates incubated under dim light (ca. 4 μE m⁻² sec⁻¹) at 23° C. Single colonies were re-streaked several times on plates containing 300 μg/mL kanamycin, before they were tested for homoplasmicity by PCR. However, the transformants remained heteroplasmic (i.e., they contained a mixture of transformed and untransformed copies of the chloroplast genome), indicating a certain level of protoxins toxicity to the host cells.

2. Inducible Expression System.

A copper-repressible system developed in the Rochaix lab (Surzycki et al., 2007), was used in which expression of the chloroplast transgene is controlled by the nuclear Cyc6:NAC2 gene (FIG. 5). With Cu²⁺ in the medium, the Cyc6:NAC2 is repressed, which destabilizes the transgene mRNA in the chloroplast. When Cu²⁺ is removed from the medium, the Cyc6:NAC2 is expressed and the NAC2 protein stabilizes the chloroplast transgene mRNA by binding to the psbD 5′ UTR region. A modification was made by the inventors to the native psbD sequence, a possible Shine-Dalgarno sequence in the 5′ UTR, GGAG, was mutated to AAAG (creating 5′ psbDm) to decrease translation in E. coli; this change should have had little or no effect in the chloroplast (Nickelsen et al., 1999). Also, to further minimize toxicity to E. coli, the Cry gene constructs were assembled in a low-copy plasmid (pET-16b).

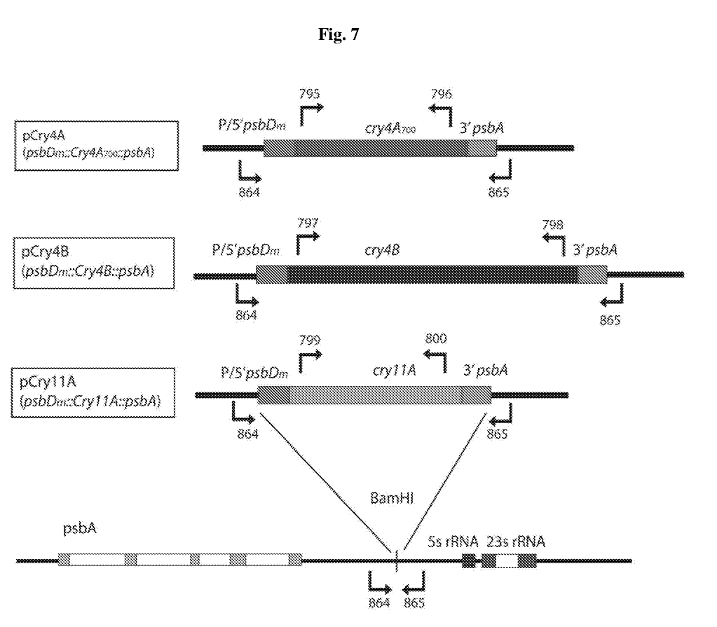

The psbDm: Cry:psbA gene constructs were cloned into an intergenic site in p322.1 (FIG. 7), and co-transformed into Ind41_18 with pB4C110, which contains a spectinomycin-resistant 16S rRNA gene; the inserts from both plasmids are from the inverted repeat region of CpDNA. Spectinomycin-resistant colonies were re-streaked on spectinomycin plates several times until they approached homoplasmicity as judged by PCR analysis of the CpDNA.

Thus, an inducible Cyc6-Nac2-psbD expression system was used for chloroplast-based expression of the exemplary protoxins. In particular, a psbD 5′ control region induced integration into the chloroplast genome of the Ind41_18 strain. See, schematics shown in FIG. 5B. For this inducible system, the 5′ control region of the chloroplast psbD gene (promoter and 5′-UTR) was fused to each Cry gene, which renders its expression dependent on the NAC2 protein (35,44).

Induced expression of Cry Genes: The host strain has the nuclear NAC2 gene under control of the Cyc6 promoter, which is repressed by Cu 2+. Removing Cu 2+ from the medium activates the Cyc6:NAC2 gene, which then activates expression of the respective Cry gene in the chloroplast. The Cry gene constructs contained the control region from psbA on the downstream side, and were inserted downstream of the rRNA genes using co-transformation with the 16S rRNA gene from a spectinomycin-resistant Chlamydomonas (19). Clones with transformed copies of chloroplast DNA (ie, homoplasmic) were obtained for each Cry gene, and grown under control (uninduced) and induced conditions.

3. Constituative Expression Systems: Attempts to Express Cry Genes Constitutively Using Rps Gene Signals.

5′ expression signals from two chloroplast ribosomal protein genes, rps4 and rps7, to drive (constitutive) Cry11Aa expression in wild-type background were contemplated for use. Ribosomal protein 5′ expression signals might direct synthesis of Cry proteins away from the thylakoid membrane, thus making it less toxic to the chloroplast. However, when Cry genes were cloned into the rps expression plasmids (P-655 for rps7 and P-657 for rps4; obtained from the Chlamydomonas Center, U. of Minnesota), Cry4Aa₇₀₀ and Cry4Ba containing clones could not be established even in E. coli. Although Cry proteins are considered to be protoxins that become fully activated in the larval gut, it is also clear they do have toxicity even as protoxins as they can damage host cells if expression is too high (Manasherob et al., 2003; Chakrabarti et al., 2006; and Chen et al., 2014).

E. PCR Screening of Chloroplast Transformants.

Cry transformants for PCR analysis were grown on a TAP-agar plate with 300 μg/mL spectinomycin, and total DNA was extracted as described by Kwon et al. (2014). To check the homoplasmicity of the chloroplast transformants, we used a set of primers (864+865) (Table 1) that amplify the integration site in CpDNA; homoplasmicity was indicated by the absence of untransformed copies of the genome. PCR with gene-specific primers for each Cry gene (795+796 for Cry4Aa₇₀₀, 797+798 for Cry4Ba, and 799+800 for Cry11Aa) (Table 1) was also performed to confirm the presence of the Cry gene. Standard PCR procedures with Taq DNA polymerase (New England Biolabs) and the manufacturer's buffer were used. The thermocycle program for these amplifications was as follows: 94° C. for 3 minutes; 33 cycles of 52° C. (1 minute), 72° C. (3.5 minutes), and 94° C. (30 seconds); 52° C. for 1 minute; and 72° C. for 5 minutes. The PCR products were analyzed on 1% agarose gels.

### RT-PCR

Total nucleic acids (TNA) were extracted as described previously (Kwon et al., 2014) from cultures (50 ml) grown in +Cu²⁺ and −Cu²⁺ TAP medium in the light until late log phase. To obtain the RNA fraction, 10 μg of the TNA preparations were treated with DNase (Turbo DNase from Ambion) in total volume of 55 μL to eliminate the DNA. 4 μL of each RNA sample was copied into cDNA using reverse transcriptase (Superscript III, Invitrogen) in a total volume of 20 μL at 65° C. (5 minutes) and internal reverse primers, 796 (Table 1) for Cry4Aa₇₀₀ and 800 (Table 1) for Cry11Aa. 1 μL of the reverse transcription reaction (cDNA) was used as the template for the PCR reaction (total volume of 25 μL) with specific primer sets: 795+796 (Table 1) for Cry4Aa₇₀₀, and 799+800 (Table 1) for Cry11Aa. Taq DNA Polymerase (New England Biolabs) was used in a standard PCR program that was the same as described herein except that the number of cycles was lowered to 24. 10 μL (out of 25 μL) of amplified cDNA was analyzed by electrophoresis in 1% agarose gels.

F. Protein Extraction from Inducible Strains.

The transformants and parental strain were grown in liquid TAP, which contains Cu²⁺ (uninducing condition), and in TAP−Cu²⁺ (inducing condition) at a light flux of ca. 40 μE m⁻² sec⁻¹ (23° C.) with shaking until late log-early stationary phase (2×10⁶-4×10⁶ cells/mL). The Erlenmeyer flasks and graduated cylinders (glass) used for the inducing culture were washed sequentially with 6 N hydrochloric acid, distilled water (7×), and MilliQ-water (3×) prior to use. For the extraction, 50 ml of culture was centrifuged and resuspended in 0.5 ml of leupeptin cocktail (20 mM Tris-HCl pH 7.4, 150 mM NaCl, 5 μg/mL leupeptin, 0.5 mM EDTA, 2 mM DTT, 1 mM PMSF; Pognonec et al., 1991); since PMSF is unstable in water, it was added right before use. Then, 0.5 ml of 2× lysis buffer (100 mM Tris-HCl pH 7.4, 4% SDS) was added, and the samples were sonicated (2×30 seconds at 80% power on ice). The lysates were rocked at 37° C. for 1 hour, centrifuged at 14,000×g (RT) for 10 min, and the supernatant was saved.

G. Western Blotting.

For the western blots, the supernatants of the cell lysates were mixed with 3×SDS loading buffer (50 mM Tris-HCl pH 6.8, 6% SDS, 30% glycerol, 0.03% bromphenol blue, 0.3 M DTT) and boiled for 3 minutes. A prestained protein ladder (PageRuler from Fermentas) was used as size markers, and the samples were separated at RT in SDS-PAGE gels (either 10% or 6%) using an acrylamide/bisacrylamide ratio of 30:0.8 and Laemmli buffer (Laemmli, 1970). The gels were 16 cm in length, 1 mm thick, the lane width was 6 mm, and 10 μg of chlorophyll (˜150 μg protein) could be loaded per lane. After the electrophoresis, the gel was soaked in cold transfer buffer (25 mM Tris base, 192 mM glycine, 5% methanol) for ˜15 min for gel equilibration. A PVDF membrane (Hybond-P, GE HealthCare) was equilibrated in 100% methanol for 10 seconds, washed with Milli-Q water for 5 minutes, and then transferred to the cold transfer buffer for ˜10 minutes. The proteins were transferred to the PVDF membrane for 1.5 hours at 12 Volts and 4° C. using a Genie Blotter (Idea Scientific) (Memon et al. 1993). The blots were stained with Ponceau S to confirm the transfer of total protein, and then blocked for 1 hour with 5% nonfat dried milk in TBS-T (Tris-buffered saline plus 0.05% Tween 20). The blots were washed 2× with TBS-T for 5 minutes each with shaking. They were probed with an anti-FLAG monoclonal antibody (M2) coupled to alkaline phosphatase (Sigma no. A9469) that was diluted 1:4,000 in TBS-T; the binding was for 1 hr at room temperature with shaking. The blots were washed 6× with TBS-T for 5 minutes each with shaking. The bound antibodies were detected using the Lumi-Phos WB Chemiluminescent substrate (Thermo Scientific) as described in the manufacturer's instructions. The chemiluminescence was detected by exposing the blots to X-Ray film, and developing them with SRX-101A (Konica Minolta). The developed films were scanned using an HP scanner and Silver Fast (LaserSoft Imaging) software. The quantification of relative western band intensity was performed using the ImageJ program (Version 1.46, National Institutes of Health, Bethesda, Md.).

H. Growth Rate Determinations.

Growth rates of the Cry transformants and the parental (Ind41_18) strain under uninduced and induced conditions were determined in liquid medium. Agar cultures were used to inoculate liquid TAP medium and the cells were grown until near-stationary phase (4×10⁶ cells/mL). Then, they were used to inoculate fresh 50-ml cultures of TAP+Cu²⁺ and TAP−Cu²⁺ to a cell concentration of 5×10⁴ cells/mL, and grown as described herein. In order to estimate growth, aliquots were removed every 12 hours for determination of total chlorophyll (Harris, 1989).

I. Bioassay for Larvicidal Activity of Cry Transformants.

Bioassays with mosquito larvae were aided by Dr. Saravanan Thangamani (University of Texas Medical Branch at Galveston, Tex.). Various concentrations of Chlamydomonas were used for the bioassay, which followed guidelines of the World Health Organization (2005) with some modifications. The Cry4Aa₇₀₀ and Cry11Aa transformants were grown under inducing and noninducing conditions, and Ind41_18 was grown under inducing conditions, as described herein, until they reached stationary phase. Then, volumes equivalent to 5×10⁶ cells, 2.5×10⁷ cells, and 5×10⁷ cells were centrifuged at 1000 rpm (GH-3.7 rotor, Beckman) for 5 minutes, and the pellets were washed with dH₂O and re-centrifuged. The final pellets were resuspended in 2 mL of dH₂O. The bioassays were performed in triplicate at 27° C., under 12 hour:12 hour light/dark cycles, and in quadrant Petri dishes (Pyrex); each quadrant contained 10 live mosquito larvae and Chlamydomonas in 5 mL of dH₂O. Two mL of concentrated Chlamydomonas cells (in dH₂O) was added to the larvae, which were in 3 mL of dH₂O, to make the final concentrations of 1×10⁶ cells/mL (1×), 5×10⁶ cells cells/mL (5×), and 1×10⁷ cells cells/mL (10×). The dH₂O was used so the algae would not grow during the assay. The larvae were transferred to dH₂O the day before the assay and starved overnight. The larvae were 3^{rd} instar Culex quinquefasciatus (C. quinquefasciatus) and 4^{th} instar Aedes aegypti (A. aegypti), and larval deaths were counted visually after 24, 48, and 72 hours with the live algae. For the determination of the median lethal concentration (LC₅₀) of the Cry11Aa transformant against 4^{th}-instar A. aegypti larvae, 10 different concentrations of induced-Cry11Aa cells were used (in triplicate) in the bioassay. These were: 2.5×10⁵ cells/mL, 3.76×10⁵ cells/mL, 5×10⁵ cells/mL, 7.5×10⁵ cells/mL, 1×10⁶ cells/mL, 1.5×10⁶ cells/mL, 2×10⁶ cells/mL, 2.5×10⁶ cells/mL, and 5×10⁶ cells/mL. The LC₅₀ was calculated with Microsoft Excel using Probit analysis (Finney, 1971).

In some cases, images of the larvae were captured using LAS EZ software and a Leica EZ4 HD stereomicroscope using transmitted (brightfield) illumination and oblique illumination.

### Example II

The following example describes initial results from methods of expressing Cry genes constitutively using psbA and rps gene signals. Attempts to express the synthetic Cry genes using pD1-KanR, p655 and p657.

Initial attempts to express the synthetic Cry genes in the Chlamydomonas chloroplast was using the pD1-KanR vector (Rasala et al., 2010). With this vector, the transgene is expressed using the 5′ and 3′ control regions of the chloroplast psbA gene, where the novel transgene of the present invention replaces the psbA gene; thus, the transformants are non-photosynthetic and kanamycin resistant. However, the transformants remained heteroplasmic despite repeated selective growth, suggesting that the proteins have some host cell toxicity. Attempts at the expression of the Cry genes using the 5′ control regions from the ribosomal protein genes rps7 and rps4, with the 3′ region coming from rbcL. However, most of these constructs were too toxic to E. coli, presumably because the rps 5′ expression signals are functional in bacteria (Fargo et al., 1998). Based on these results, an inducible chloroplast expression system was then used which was successful in expressing novel Cry genes of the present inventions.

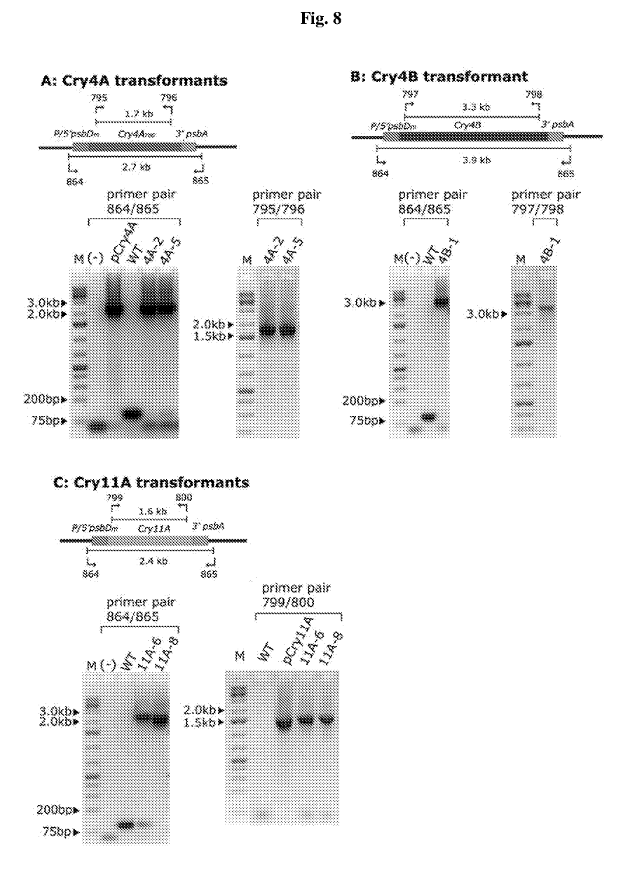

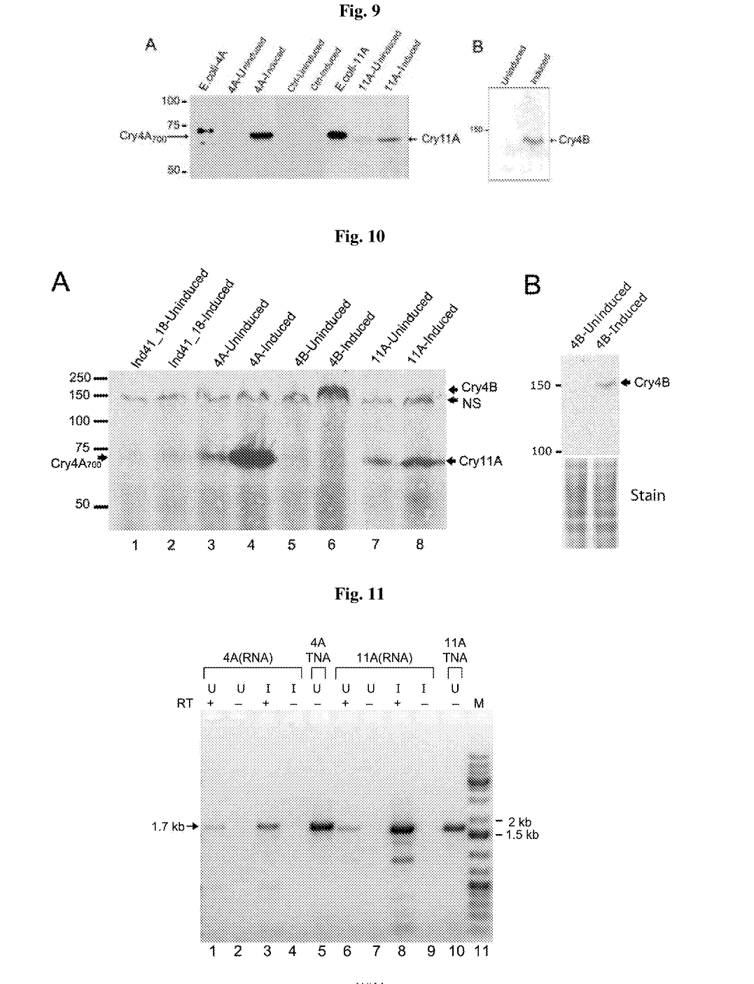

Exemplary results expressing CRY proteins in the inducible strains of Chlamydomonas are described as follows. FIG. 8 shows the PCR analysis used to evaluate transformants. Primer pair 864/865 amplifies the integration site in CpDNA and was used to judge the homoplasmicity of transformants. The 864/865 pair gives a small product (˜100 bp) from genome copies that have no Cry gene insert but a large product from copies that have been transformed; the absence of the small product indicates that all copies have been transformed (i.e., homoplasmic). Also, internal primer pairs were used to verify the presence of the specific Cry gene (795/796 for Cry4Aa₇₀₀, 797/798 for Cry4Ba, and 799/800 for Cry11Aa), and in every case they gave the correct size product (FIG. 8). The results indicate that one of the two Cry4Aa₇₀₀ transformants is homoplasmic (4A-2 in FIG. 8), while the other (4A-5) still had some untransformed copies. The Cry4Ba transformant was homoplasmic (4B-1 in FIG. 9), whereas one of the two Cry11Aa transformants is homoplasmic (11A-8), but the other (11A-6) contained untransformed CpDNA copies. It should be noted that the CpDNA in Ind41_18 and wild-type (WT) are the same in that region of the genome.

### Example III

The following example describes exemplary characteristics of, methods of culture and strains of Chlamydomonas reinhardtii used during the development of the present inventions.

A. Chlamydomonas.

Chlamydomonas reinhardtii is a unicellular, eukaryotic green alga. Chlamydomonas has a single chloroplast that is 40% of the cell volume (Rochaix, 1995) such that the use of a gene gun on the whole organism may result in transformation of the chloroplast genome. Chlamydomonas also has two anterior flagella that are used for motility, and in mating (FIG. 4) (Harris, 1989). Doubling time is short (usually 8-12 hours), and large-scale culture costs are low (Harris, 2001).

Moreover, recombinant proteins can be targeted to different organelles (nucleus, mitochondria, ER, and chloroplast), or secreted out of the cell (Lauersen et al., 2013; Rasala et al., 2014a). Also, C. reinhardtii, like many algae, is classified by U.S. FDA as a GRAS (generally regarded as safe) organism (Specht et al., 2010). The chloroplast has μ80 copies of a genome that is ˜200 kb, and contains two inverted repeats of 21.2 kb and two single-copy regions of 80 kb and 78 kb (Maul et al., 2002). The genome encodes 99 genes, mostly involved in photosynthesis, transcription, and translation (Harris et al., 2009). The chloroplast genome contains the potential for high levels of foreign (heterologous) gene expression, typically an absence of gene silencing, has unique expression signals, and restrictive (uniparental) inheritance as advantages for chloroplast engineering (Grant et al., 1980; Cerutti et al., 1997; Manuell and Mayfield, 2006). The manipulation of the C. reinhardtii chloroplast genome using biolistic bombardment is well established (Boynton et al., 1988); transgenes are inserted in a site-specific manner by homologous recombination, and are generally stable unless they are highly toxic to the host (Surzycki et al., 2009; Rasala and Mayfield, 2014b). Moreover, the chaperones and protein disulfide isomerases in the chloroplast of C. reinhardtii are capable of folding some complex proteins (Rasala and Mayfield, 2014b).

There are two mating types (+ and −), as it can reproduce sexually or asexually (Pröschold et al., 2005). During sexual reproduction, which is critical for its survival in the wild, the vegetative cells differentiate into gametes, mate, and form a diploid zygote (Harris, 2001). The zygote is tough and resistant to hostile conditions and predation, but when conditions are better it germinates and divides into haploid zoospores (vegetative cells).

Chlamydomonas cells are haploid and can grow in the laboratory on a simple medium of inorganic salts, using photosynthesis to provide energy. They can also grow in total darkness if acetate is provided as an alternative carbon source. When deprived of nitrogen, haploid cells of opposite mating types can fuse to become a diploid zygospore i.e. zygote as described above. When conditions improve (or when nitrogen is restored to the culture medium along with light and water), the diploid zygote undergoes meiosis and releases four haploid cells that resume the vegetative life cycle.

B. Chlamydomonas Strains and Media.

The Ind41_18 strain of C. reinhardtii was obtained from J-D Rochaix (U. of Geneva, Switzerland). The wild-type 2137 C. reinhardtii strain (CC-1021) was from the Chlamydomonas Culture Center. The cultures were maintained by periodic transfer to fresh plates of Tris-acetate-phosphate (TAP) medium (Gorman and Levine, 1965) that were kept in the light at 23° C. TAP medium was also used as the +Cu²⁺ medium (TAP+Cu²⁺), and TAP minus copper (TAP−Cu²⁺) was made by removing copper from the Hutner's trace elements solution. It was prepared as described by Quinn and Merchant (1998) and Harris (1989). A mixture of ZnSO₄.7H₂O, H₃BO₃, MnCl₂.4H₂O, CoCl₂.6H₂O, (NH₄)₆Mo₇O₂₄.4H₂O, and FeSO₄.7H₂O was boiled, and then the EDTA solution was added. CuSO₄.5H₂O, which was used for the normal Hutner's trace element solution, was not added for the +Cu²⁺ medium. After cooling to 70° C., the pH was adjusted to 6.7 by adding hot 20% KOH. After adjusting the final vol to 1 L with MilliQ-water, the solution was allowed to stand for 1-2 weeks with daily shaking. During this time, the solution changed from orange-red to burgundy red. Liquid culture was in flasks that were ca. 40% full and mixed continuously on an orbital shaker at 125 rpm. Cell counts were made with a hemacytometer after killing the cells with iodine (5% (w/v) I₂, 10% (w/v) KI). Also, for the growth rate tests, total chlorophyll was used to estimate the number of cells/mL using the reference value of 4 mg chlorophyll per 1×10⁹ cells (Harris, 1989).

### Example IV

The following example describes testing the viability and larvicidal activity of transfected strains of Chlamydomonas reinhardtii induciably expressing, individually, CRY4A-700, Cry11A, and Cry4B.

The synthetic, codon-adapted reverse engineered genes based upon native Cry4Ba (˜130 kDa) and Cry11Aa (75 kDa) proteins (i.e. amino acid sequences) and Cry4Aa, having the first 700 amino acids, which contain the toxin activity (7) were each individually transformed into a Chlamydomonas Ind41_18 strain.

Data on Expressing the Bti Cry Genes in the Chloroplast: These results, see, for example FIG. 6, show that a protein of the expected size was induced for each of the three Cry genes with the relative order of expression Cry4A 700>Cry11A>Cry4B at an approximate ratio of 12:3:1. This relative order of amounts of protein expression was confirmed by obtaining a gel blot with the three proteins on the same gel.

Western blots of total protein probed with the anti-Flag antibody showed the accumulation of all 3 Cry proteins (FIG. 6), with Cry4A700 showing the highest level. The proteins produced in E. coli that were included on the Cry4A 700 and Cry11A blot (FIG. 6A) are slightly larger because of a His-tag at the N-terminus.

These transformed Chlamydomonas as strains based upon which transgene they expressed, were tested for larvicide activity on mosquito larvae. Both the Cry4A 700 and Cry11A transformants were lethal to A. aegypti and Culex sp. larvae. The Cry11A transformant was at least 2-fold more toxic than the Cry4A 700 strain to A. aegypti, despite having approx. 4-fold less Cry protein; this is consistent with the known toxicity of native Cry11A produced by Bti bacteria compared to native Cry4A. A. Further, there was little or no evidence of inhibition of growth of Chlamydomonas strains after inducing transgene expression of the Cry proteins, at least under these conditions and with Ind41_18 as the host strain.

The functionality of the Cry4A700 and Cry11Aa proteins was indicated by live cell bioassays that employed Aedes aegypti and Culex quinquefasciatus. Representative results with third instar A. aegypti larvae are shown in FIG. 7. As the data show, both Cry genes were toxic to the larvae, with the Cry11Aa strain being more potent despite having a lower level of Cry protein.

The greater toxicity of Cry11Aa was reported in other systems (10) which indicates that the protein expressed in the choloplast organelle is folding correctly. The expression of the truncated form of Cry4Aa was relatively high (about 0.1% of total protein). Thus the inventors are contemplating truncating Cry4Ba and other Cry proteins in order to find out if the truncated versions will have increased expression.

A. Western Blot Analysis of Cry Protein Accumulation.

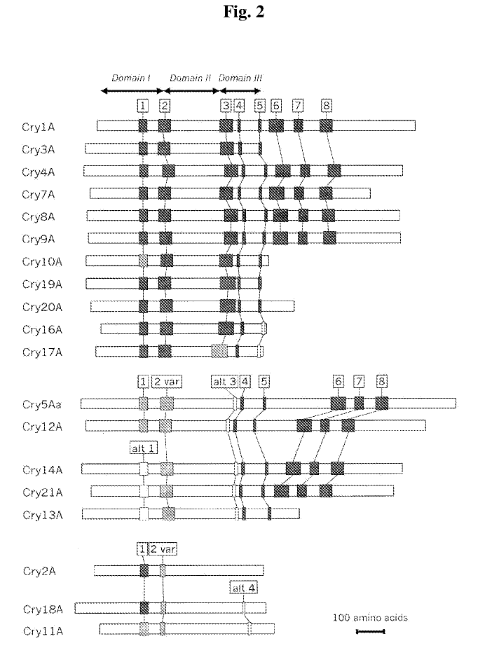

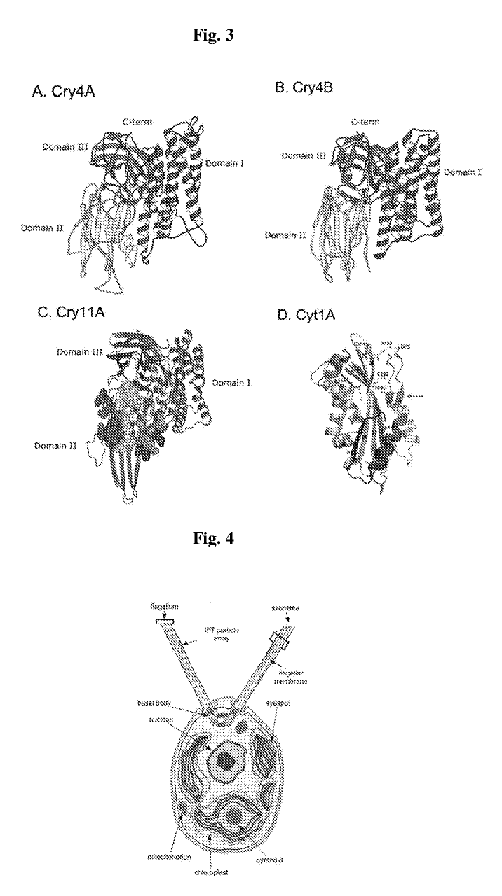

Accumulation of the Cry proteins in the transformants grown with Cu²⁺ (Uninduced) and without Cu²⁺ (Induced) was assessed using western blotting of total cell protein with an anti-Flag antibody (FIG. 10). It should be noted that both of the Cry4Aa₇₀₀ transformants (FIG. 8) gave similar results, as did both of the Cry11Aa transformants (FIG. 8), so results with the homoplasmic Cry4Aa₇₀₀ and Cry11Aa transformants (4A-2 and 11A-8, respectively) are shown in FIG. 2. The left panel is from a 10% gel, and contained all 3 types of transformants, whereas the right panel is from an 6% gel, which was used to better separate the very large Cry4Ba protein (˜146 kDa) from a non-specific protein band (NS) that light up with the Flag antibody (left panel). Proteins of expected sizes were obtained (or increased) under the induced conditions for Cry4Aa₇₀₀ (74 kDa), Cry4Ba (146 kDa), and Cry11Aa (73 kDa). There was also significant accumulation of Cry11Aa, and to a lesser extent Cry4Aa₇₀₀, in the uninduced condition. However, the induction of Cry4Aa₇₀₀ was quite strong (6-10-fold), whereas the increase in Cry11Aa under induction was 2-2.5 fold. Quantification of three different blots provided an estimate of the relative expression of the Cry proteins under induction conditions as 3.5:1:0.75 for Cry4Aa₇₀₀:Cry11Aa:Cry4Ba.

B. Western Blot Analysis of Cry Transformants with the Anti-Flag Antibody.

(A) Total cell protein fractions (10 μs chlorophyll, ˜150 μs protein) were separated on a 10% polyacrylamide gel, blotted and probed with a monoclonal anti-Flag antibody. The Chlamydomonas strains were: Ind41_18, parental; 4A, Cry4Aa₇₀₀ transformant 4A-2; 4B, Cry4Ba-1 transformant 4B-1; 11A, Cry11Aa transformant 11A-8. Each strain was grown under uninduced and induced conditions for ˜72 hours. The non-specific (NS) band at ˜145 kDa in all the lanes serves as a loading control. (B) Total cell protein fractions (˜150 μg protein) from the 4B-1 transformant, grown as indicated, were separated on a 6% polyacrylamide gel. Duplicate lanes were either stained with Coomassie blue (bottom panel) to check the loading, or blotted and probed with the anti-Flag antibody (top panel).

C. RT-PCR Analysis of Cry4Aa700 and Cry11Aa Expression.

Although Cry4Aa₇₀₀ and Cry11Aa are similar-sized proteins, the Cry4Aa₇₀₀ protein level under inducing conditions was 3-4-fold higher than Cry11Aa, so we decided to examine the mRNAs by semi-quantitative RT-PCR. FIG. 11 shows that both mRNAs were present without induction, but that both also increased substantially (3-5-fold) under induction conditions. The presence of the mRNAs without induction suggests that the absence of the NAC2 protein is not totally destabilizing for the mRNAs; moreover, it explains the presence of the Cry11Aa protein without induction. On the other hand, the results indicate a lack of correlation between the psbDm:Cry11Aa:psbA mRNA and the Cry11Aa protein with the mRNA induction being much stronger than the protein induction (˜5-fold versus 2-fold). This suggests that Cry11Aa expression is limited at the level of translation or protein stability, at least under inducing (−Cu²⁺) conditions.

RT-PCR analysis of the Cry4Aa₇₀₀-2 (4A) and Cry11Aa-8 (11A) transformants. An equal amount of RNA from cultures grown for 72 hours under uninduced (U) and induced (I) conditions was used for reverse transcription with gene-specific primers; 796 for Cry4A₇₀₀ and 799 for Cry11A. The resulting cDNAs were amplified using primers 795+796 for Cry4Aa₇₀₀ and 799+800 for Cry11Aa. Reactions without reverse transcriptase in the RT step served as negative controls (lanes 2, 4, 7, 9). Also, PCR reactions with total nucleic acids (TNA) from both strains served as positive controls for the PCR step (lanes 5 and 10). Lane M contained size markers, and the gel image was inverted. RT, reverse transcriptase

D. Growth Rates.

Growth rates of the Cry gene transformants under induced and uninduced conditions were determined and found to be similar indicating that there was little toxicity of the Cry protoxins to the host, at least with these constructs.

E. Effect of Inducing Cry Genes on Growth Rates.

To test for toxicity of the accumulated Cry proteins to Chlamydomonas cells, the growth rates of the transformants (and parental strain) under −Cu²⁺ (Induced, I) and +Cu²⁺ (Uninduced, U) conditions were examined (FIG. 12). Surprisingly, the growth curves obtained under both conditions were quite similar for the Cry4Aa₇₀₀, Cry4Ba, and Cry11Aa transformants, suggesting that the proteins are not highly toxic when expressed under these conditions (i.e., with the psbDm control region, in the Ind41_18 host strain, and in minus-Cu²⁺ medium).

In summary, synthetic genes for three major Cry proteins of the Bti endotoxin were inducibly expressed in the Chlamydomonas chloroplast, with expression levels from high to low as Cry4A 700>Cry11A>Cry4B.

The Cry4A 700 and Cry11A strains (induced) were tested in live-cell bioassays with mosquito larvae (Aedes aegypti and Culex sp.) and both were lethal; the LC50 of the Cry11A strain against A. aegypti was 3.3×10⁵ cells/mL using Probit analysis.

F. Larvicidal Activity of the Cry Inducible Transformants.

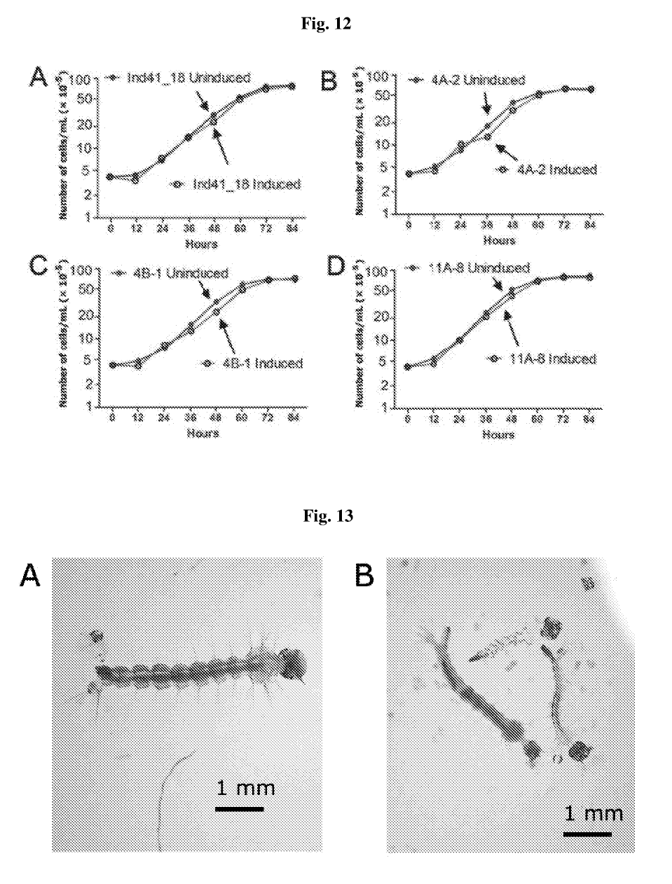

To test for activity of the Cry proteins/transformants, live cell bioassays with mosquito larvae were performed. We used 4^{th} instar larvae of A. aegypti and 3^{rd} instar larvae of C. quinquefasciatus—the larval stages were identified by morphology—and dH₂O was used as the medium, so the algae would not grow, but remain alive. When the larvae were raised on untransformed Chlamydomonas cells (Ind41_18), they were very active and developed into pupae and adults, confirming that Chlamydomonas can be used as sole food source (Marten, 1986; Kaufman et al., 2006). Larvae feeding on induced Cry4Aa₇₀₀ and Cry11Aa transformants became sluggish and most eventually died; the dead larvae had dark bodies with poorly defined abdominal segments (FIG. 13), and did not respond to physical stimuli.

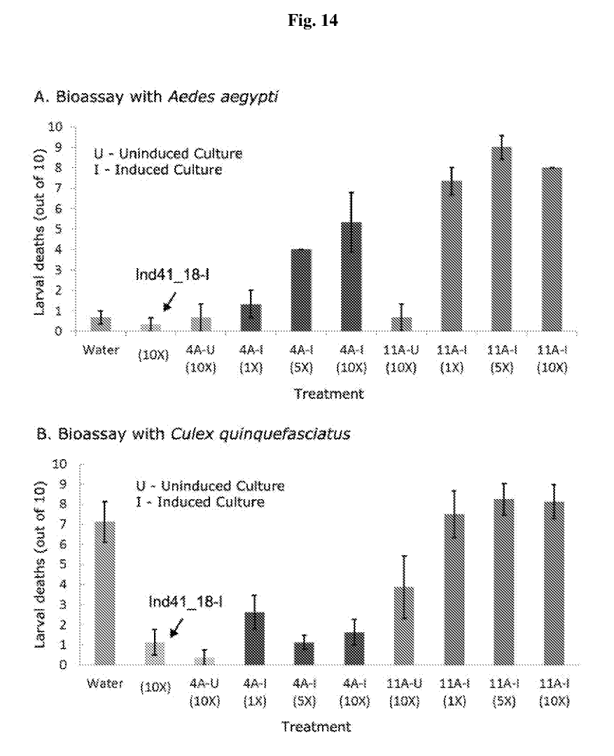

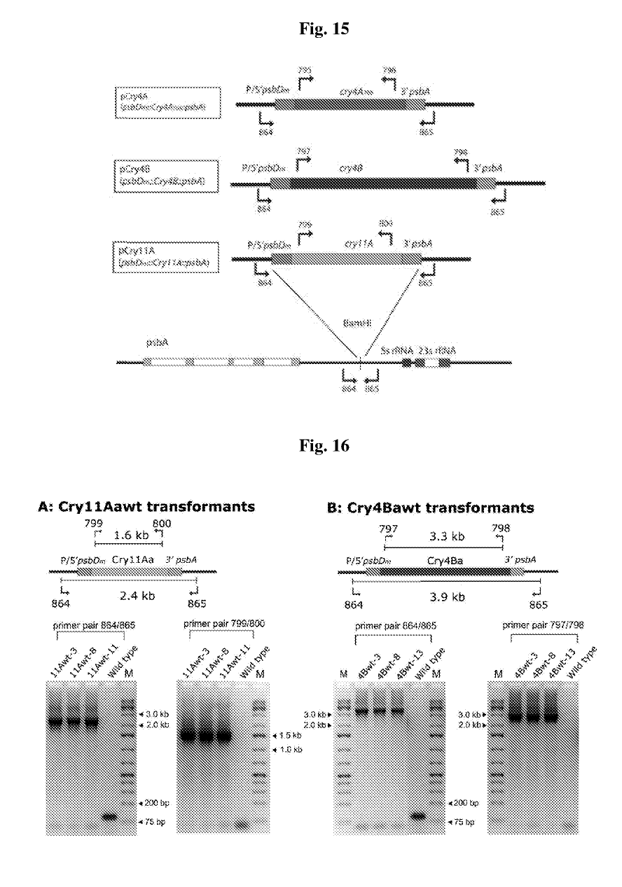

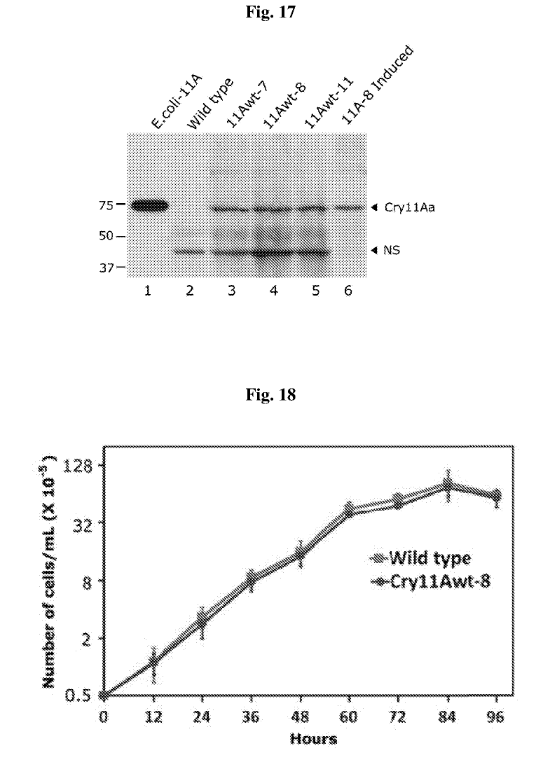

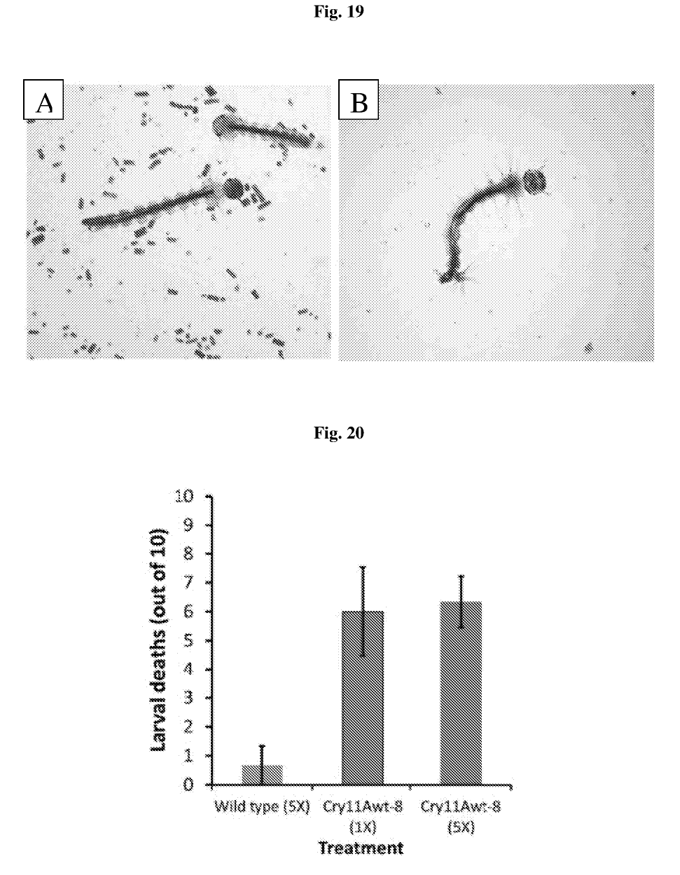

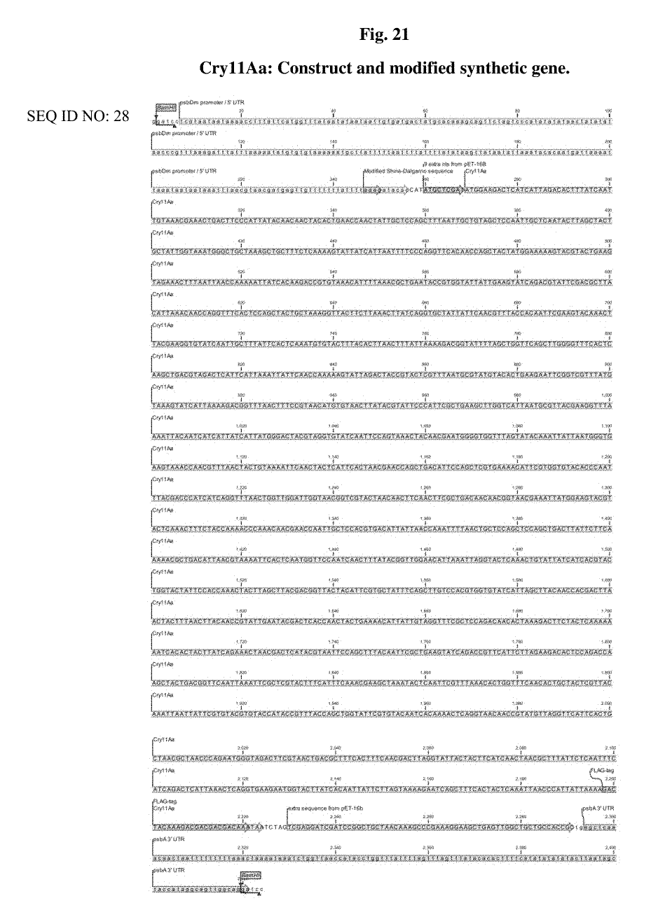

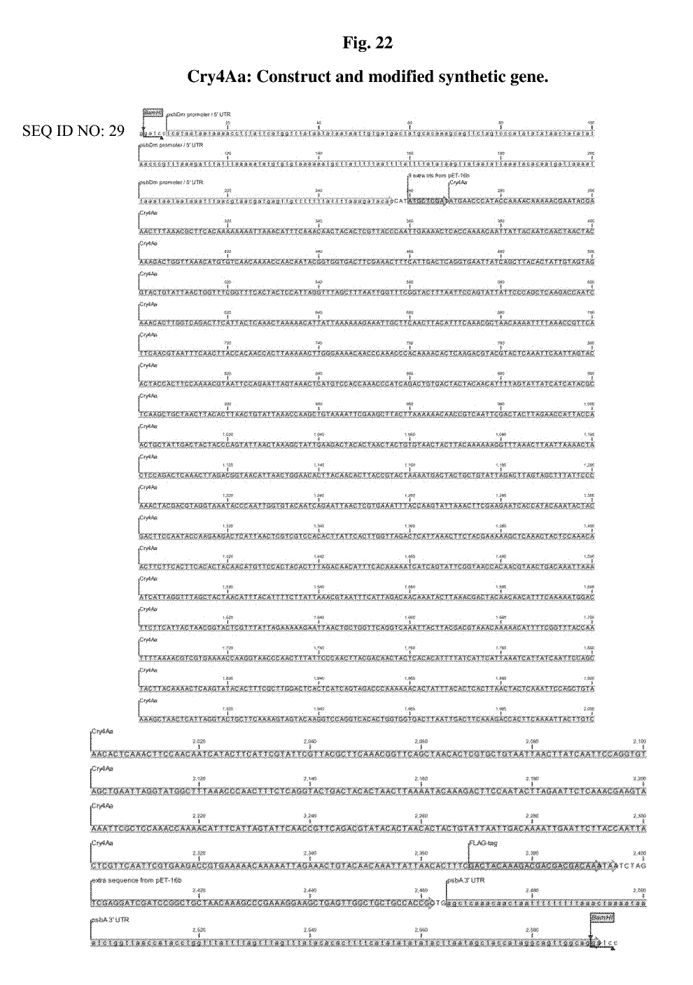

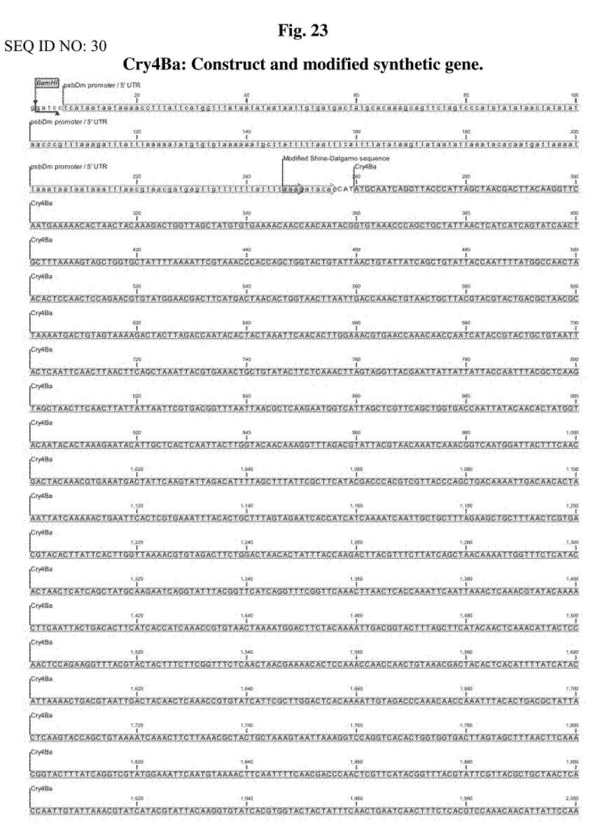

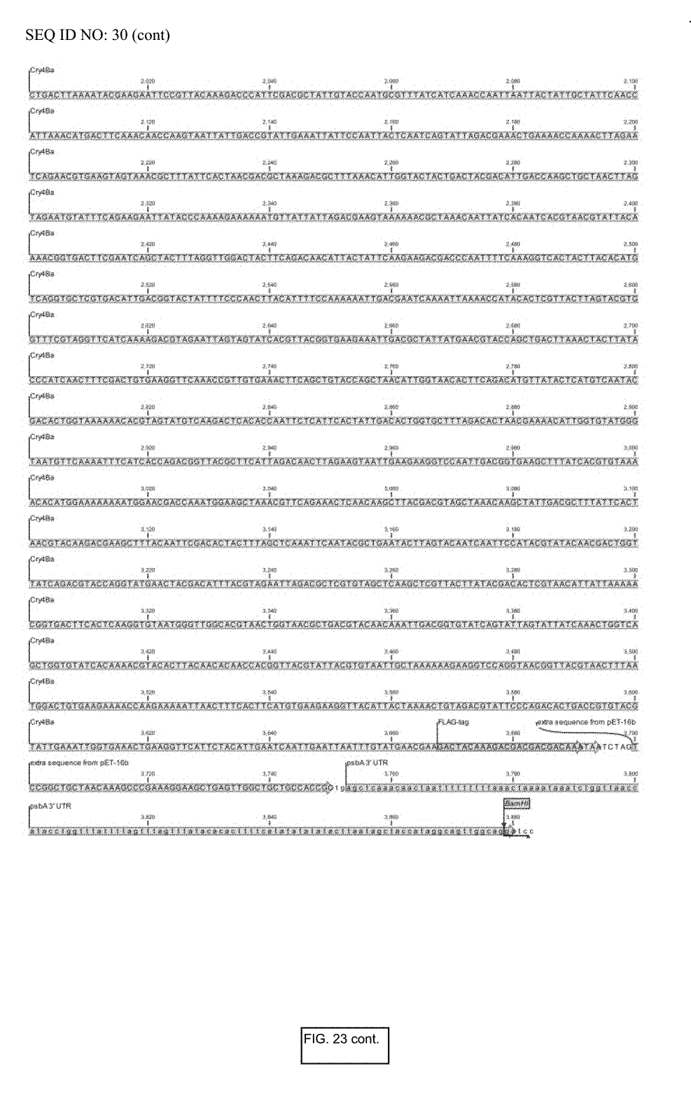

FIGS. 14A and 14B shows the bioassay results with the Cry4Aa₇₀₀ and Cry11Aa transformants in terms of larval deaths (out of 10) after 48 hours for A. aegypti (FIG. 14A) and C. quinquefasciatus (FIG. 14B). Initial tests with the Cry4Ba transformant showed low toxicity against A. aegypti. Cry4Ba is known to have low toxicity against Culex sp. (Angsuthanasombat et al., 1992; Delécluse et al., 1993). As FIG. 14A shows, both the Cry4Aa₇₀₀ (4A) and Cry11Aa (11A) transformants were lethal to A. aegypti larvae, with Cry11Aa exhibiting ˜3-fold greater toxicity at a cell concentration of 1×10⁶ cells/mL (=1×). The relatively low lethality of uninduced Cry11Aa at the 10× cell concentration—compared with the induced cells at the same cell concentration (10×)—was somewhat unexpected, since the uninduced cells contained 2-2.5-fold less Cry11Aa than the induced cells. It should be noted, however, that the comparative effects of the uninduced and induced Cry11Aa cells on the C. quinquefasciatus larvae (compare 11A-U (10×) with 11A-I (10×) in FIG. 14B) were more consistent with the western blot data than were the results with A. aegypti. As with A. aegypti, however, the Cry11Aa transformant was more lethal to the C. quinquefaciatus larvae than the Cry4Aa₇₀₀ transformant (FIG. 14B). The data also provide evidence of toxicity inhibition at the higher algal cell numbers (5× and 10×), and this effect is probably analogous to the suppressing effect that food has on Bti toxicity (Becker and Margalit, 1993; Saiful et al., 2012). It is also apparent that the C. quinquefasciatus larvae do not survive in dH₂O (with no algae) as well as the A. aegypti larvae, which is consistent with the known abilities of these species to resist starvation (i.e., A. aegypti is much more resistant to starvation than C. quinquefaciatus). To estimate LC₅₀ for the induced Cry11Aa transformant, a more extended series of cell concentrations were used in the bioassay with 4^{th} instar A. aegypti larvae. After Probit analysis of the data, the LC₅₀ was found to be 3.3×105 cells/mL.

### Example V

The following example describes producing and testing the viability/larvicidal activity of transfected strains of wild-type Chlamydomonas reinhardtii grown in the laboratory that were constituatively expressing, individually, either Cry11A or Cry4B.

Homoplasmic transformants of viable Chlamydomonas constiutively expressing Cry4Ba and Cry11Aa were made. A wild-type strain of C. reinhardtii, 2137 (CC-1021 wild type mt+), was obtained from the Chlamydomonas Center (U. of Minnesota, USA). Strains were grown in TAP medium in the light (40 μE m⁻² sec⁻¹) at 23° C. with shaking.

A. Transformation of Wild-Type And DNA Analysis

The same contructs used in Examples I, II, III and IV, were transfected into a wild-type Chlamydomonas strain, where Cry gene expression should be driven by light (21), i.e. constiutive expression from light sensitive plasmid promoters. Thus, Cry4Aa₇₀₀, Cry4Ba, and Cry11Aa novel genes were ligated to psbDₘ and psbA regulatory regions, and integrated into the chloroplast transformation vector p322.1.

FIG. 7 shows the Cry plasmids that were co-transformed into wild-type C. reinhardtii, with the site of integration between the psbA and 23S rrn genes. Each novel gene was co-transformed with pB4CC110, which confers spectinomycin resistance, as described in herein. Transformants were selected on 100-μg/mL spectinomycin. After primary selection, the transformants were restreaked on 300-μg/mL spectinomycin plates until they became homoplasmic, as estimated from PCR amplification results with primer pair 864/865 bordering the integration site. PCR was also performed to check the integration of Cry constructs. Homoplasmicity is indicated by the absence of the ˜100 bp product. DNA extraction, PCR primers, and amplification conditions were the same as described in herein. Cell number for the wild-type transformants was estimated from total chlorophyll using the reference value of 4 mg chlorophyll per 1×10⁹ cells (Harris, 1989).

As shown in FIG. 8, at least 3 transformants were obtained for Cry11Aa and Cry4Ba, where copies of the CpDNA have an integrated Cry gene. PCR with internal primer pairs (799/800 for Cry11Aa and 797/798 for Cry4Ba) confirmed the presence of the respective Cry gene in each case (FIG. 8). Homoplasmic transformants were not Detected Containing The Cry4Aa₇₀₀ Plasmid.

B. Protein Extraction and Western Blotting.

Cell cultures in late log phase (2-4×10⁶ cells/mL) were harvested, solubilized with SDS and sonication, and subjected to SDS-PAGE on 10% acrylamide gels. The proteins were electrotransferred to a PVDF membrane, and detected with a monoclonal anti-Flag antibody as described herein.

### Chlorophyll Measurement and Cell Number Conversion

Total chlorophyll was measured by harvesting the cells from 1 mL of culture using centrifugation at 10,000×g for 5 minutes, and then extracting the pellet with 1 mL of 95% EtOH. After centrifuging at 10,000×g for 2 minutes, the supernatant was removed, and its absorption was read at 665 nm and 649 nm. Total chlorophyll, in μg/mL culture, was calculated as described in Windermans and De Mots (1965).

C. Bioassay for Larvicidal Activity.

The bioassay was performed with 4^{th} instar Aedes aegypti larvae as described herein. Ten larvae (per assay) were fed live wild-type and Cry transformant cells in dH₂O, and larval mortality was checked every 24 hours. When desired, images of the larvae were captured using LAS EZ software and a Leica EZ4 HD stereomicroscope.

FIG. 9A shows that Cry11Aa transformants have at least as much Cry11Aa as do induced cells of the inducible Cry11Aa transformant. In particular, PCR analysis of chloroplast DNA from total DNA extracted from three Cry11A wild-type transformants was used. PCR with primers that flank the integration site in CpDNA (864/865) and primers that are internal to Cry11A (799/800) amplified this section of the transgene. A reaction with wild-type (2137) DNA is also shown for comparison. Size markers are also indicated (lane M).

Western blot analysis of 3 Cry11Aa wild-type transformants are shown in FIG. 9B. The Cry11A wild-type transformants and the untransformed wild-type (Ctrl) strain were grown in continuous light, and equal numbers of cells (based on hemacytometer counts) were loaded on the 10% acrylamide gel. An inducible Cry11A transformant was also included for comparison, though it was loaded at ˜50% of the wild-type strains. Thus, Bti Cry genes were stably expressed in the chloroplast of viable Chlamydomonas. A larval bioassay of a Cry11A wild-type transformant (Cry11Awt-8) was tested with A. aegypti 4^{th}-instar larvae. Ten larvae were used per assay (n=3), which was performed with live algal cells in water. The data are from 48 hrs of incubation.

In summary, Cry11Aa-producing strains were established with wild-type Chlamydomonas, producing constitutively expressed toxin for larvicidal-Chlamydomonas strains. These homoplasmic transformants were stable and lethal to A. aegypti larvae. Results show a high level of larval death.

TABLE 2
Derived Genes And Summary Of Results.

| | | Protein | Protein |
|---|---|---|---|
| | % identity of | Expression in | Expression in |
| | Codon Modified | Induced | wild-type |
| | gene to NCBI Bti | Chlamydomonas | Chlamydomonas |
| Cry gene | gene | strain | strain |
| Cry11Aa | 76% identical | YES | YES |
| | (24% different) | | |
| Cry4Aa | 77% identical | YES | NO |
| | (23% different) | | |
| Cry4Ba | 78% identical | YES | NO |
| | (22% different) | | |

### Example VI

The following example describes expression of Cyt1Aa.

Cyt1Aa is a Bacillus thuringiensis israelensis toxin protein that is contemplated to synergistically increase the lethality of the Cry proteins when expresed in Chlamydomonas reinhardtii chloroplasts. It has weak cytolytic activity against certain cell types, which depended on the phospholipids in their cell membrane (Federici et al., 2003). In other systens, toxicity of Cyt1Aa, without other toxins, against mosquito larvae is weak, compared to the Bti Cry proteins, Cry4Aa, Cry4Ba, and Cry11Aa. In contrast, Cyt1Aa suppresses the development of resistance in mosquito larvae exposed to Bti toxins. Toxicity of Cyt1Aa is mediated by a toxin-lipid interaction rather than by the toxin-receptor interaction that mediates the toxicity of Cry proteins (Butko, 2003). Moreover, Cyt1Aa can act as a receptor for Cry4Ba and Cry11Aa.

Hence, we contemplated expression of Cyt1Aa in the chloroplast of Chlamydomonas reinhardtii, in order to complement our success in expressing the Cry protein genes, and because it may be provide the best possible mosquito larval biocontrol organism.

The Cyt1Aa DNA sequence (774 bp) of B. thuringiensis israelensis (NCBI NC_010076.1) was optimized using the program Optimizer (Puigbo, et al., 2007) and a codon-usage table of the chloroplast of Chlamydomonas reinhardtii (Nakamura, et al., 2000). A FLAG tag sequence was added to the 3′ end of the Cyt1Aa sequence (FIG. 24). A nucleotide at position of 296 was changed from adenine to thymine for the ease of cloning by removing NdeI restriction site. The codon-optimized coding sequence: DNA sequence of the codon-optimized Cyt1Aa gene with FLAG tag is SEQ ID NO:18.

Cyt1Aa Condon-Optimized with FLAG Tag: SEQ ID NO:18:

ATGGAAAATTTAAATCATTGTCCATTAGAAGATATTAAAGTTAATCCAT
GGAAAACACCACAATCAACAGCTCGTGTTATTACATTACGTGTTGAAGA
TCCAAATGAAATTAATAATTTATTATCAATTAATGAAATTGATAATCCA
AATTATATTTTACAAGCTATTATGTTAGCTAATGCTTTTCAAAATGCTT
TAGTTCCAACATCAACAGATTTTGGTGATGCTTTACGTTTTTCAATGCC
AAAAGGTTTAGAAATTGCTAATACAATTACACCAATGGGTGCTGTTGTT
TCTTATGTTGATCAAAATGTTACACAAACAAATAATCAAGTTTCAGTTA
TGATTAATAAAGTTTTAGAAGTTTTAAAAACAGTTTTAGGTGTTGCTTT
ATCAGGTTCAGTTATTGATCAATTAACAGCTGCTGTTACAAATACATTT
ACAAATTTAAATACACAAAAAAATGAAGCTTGGATTTTTTGGGGTAAAG
AAACAGCTAATCAAACAAATTATACATATAATGTTTTATTTGCTATTCA
AAATGCTCAAACAGGTGGTGTTATGTATTGTGTTCCAGTTGGTTTTGAA
ATTAAAGTTTCAGCTGTTAAAGAACAAGTTTTATTTTTTACAATTCAAG
ATTCAGCTTCATATAATGTTAATATTCAATCATTAAAATTTGCTCAACC
ATTAGTTTCATCATCACAATATCCAATTGCTGATTTAACATCAGCTATT
AATGGTACATTAGACTACAAAGACGACGACGACAAATAA.

The codon-optimized coding sequence SEQ ID NO:18 was used as the basis for designing primers for gene assembly, which were 50 nucleotides in length and contained 25-nucleotide overlaps with the flanking primers in the opposite orientation. Cyt1Aa was synthesized using those primers and DNA shuffling method (Stemmer, (1994) DNA shuffling by random fragmentation and reassembly: in vitro recombination for molecular evolution. Proceedings of the National Academy of Sciences of the United States of America 91, 10747-10751) (FIG. 25). The mixture of primers were elongated and amplified using the Phusion DNA polymerase (NEB). The first product was purified using GenElute™ PCR Clean-Up (Sigma-Aldrich) and used for the template DNA of the second PCR with outside primers to produce only full-length Cyt1Aa. The in vitro-synthesized Cyt1Aa was ligated into pBluescript for cloning using Nde I (on the 5′ side) and Xba I sites (on the 3′ side). For recloning, the Cyt1Aa was excised from pBluescript using Xba I, blunting with the Klenow DNA polymerase, and then digestion with Nde I. To produce pET-Cyt1Aa, the Cyt1Aa was ligated to the pET-16B vector that had been cut with Bam HI (on the 3′ side), blunted with the Klenow DNA polymerase, and digested with Nde I (on the 5′ side). The nucleotide sequence of pET-Cyt1Aa was confirmed by Sanger sequencing (University of Texas at Austin DNA Facility).

For chloroplast expression, the psbaₘ 5′ region and the psbA 3′ region used for the Cry genes were ligated to the 5′ and 3′ ends of Cyt1Aa in pET-Cyt1Aa. Then, the psbaᵣCyt1Aa-psbA gene construct, which had been excised with BamHI (on both sides), was cloned into the chloroplast expression vector, p322-483aadA, yielding plasmid pCyt1Aa (FIG. 26). The p322-483aadA vector had been generated by inserting the recyclable selectable marker for the Chlamydomonas chloroplast, 483aadA (Fischer, et al., 1996), into plasmid p322. The pCyt1Aa DNA was bombarded into the chloroplast of a wild-type strain of Chlamydomonas reinhardtii, CC1690, as described for the Cry gene expression.

Methods of protein extraction and analysis are briefly described. For the extraction of total cellular protein, 50 mL (or 30 mL) of transformed Chlamydomonas culture was pelleted by centrifugation at 2,000 rpm for 10 minutes (Heraus Centrifuge) at room temperature. The pellet was resuspended in 1 mL of lysis buffer (100 mM Tris-HCl pH 8.5, 100 mM DTT, 7 mM Benzamidine, and 5 mM EDTA pH 8.0). For the protein gel, 0.6 mL of cell lysate was treated with 0.4 mL of LDS buffer (5% lithium dodecylsulfate, 30% sucrose, and 0.025% Bromophenol blue). The preparation was stored at −70° C. (in 60 mM Tris-HCl pH 8.5, 60 mM DTT, 4.2 mM Benzamidine, 3 mM EDTA, 2% lithium dodecylsulfate, 12% sucrose, 0.01% bromophenol blue). Aliquots were loaded onto 10% polyacrylamide-SDS gels, and after separation, the proteins were electrotransferred to a PVDF membrane. The protein blots were probed with an anti-FLAG monoclonal antibody coupled to alkaline phosphatase, and detected with a chemiluminescent substrate and X-Ray film.

The total protein concentration of the cell lysates was determined with the Bradford reagent (Bio-Rad Protein Assay, Bio-Rad). To prepare the protein and remove the chlorophyll, the transformed Chlamydomonas culture cells were pelleted by centrifugation at 16,000 rpm for 5 min at room temperature, followed by resuspension in 90% acetone. The samples were then mixed, incubated for 2-3 min and centrifuged. The protein pellet was resuspended in Tris-HCl pH 8.0, 1% SDS and heated at 60° C. for 2-3 min. The samples were subject to the Bradford (Bio-Rad Protein Assay, Bio-Rad) using IgG for the standard curve.

Chlamydomonas transformants are homoplasmic, that is they have transformed copies of the chloroplast genome; i.e., there are no untransformed copies of CC1690 chloroplast DNA left as evidenced by the absence of the small PCR product that is indicative of CC1690 DNA (FIG. 27 panel C). FIG. 27 shows exemplary PCR analysis of DNA from three Cyt1A chloroplast transformants.

To visualize the Cyt1A protein in the algae, western blotting was used with a monoclonal antibody to the FLAG tag at the end of the protein. The western blot in FIG. 28 shows exemplary results of one of the three Cyt1A transformants. The blot shows a strong specific protein band of the estimated size for the Cyt1A protein in the transformant.

### Example VII

The following example describes contemplative methods for increasing larvicidal activity of Chlamydomonas reinhardtii. In particular, these strategies are generate are contemplated for use with Cry11Aa and Cry4Aa₇₀₀ transformants of wild-type Chlamydomonas.

A. Boosting Larvicidal-Chlamydomonas reinhardtii Activity.

Methods are contemplated for increasing the lethality of Cry11Aa expressing Chlamydomonas strains to mosquito larvae (at least above LC₅₀ is ˜3-5×10⁵ cells/mL). In part so that lower cell numbers would provide an effective larval control. A contemplated target larval lethality is at least ˜10⁴ cells/mL. However, sublethal doses of Bti may harm mosquito larvae enough to prevent maturation into adults (Aïssaoui and Boudjelida, 2014), thus additional contemplative measures might be used to reduce toxicity to the host. In other words, one contemplate goal is to increase the toxicity of the Bti-Chlamydomonas about 50-fold in viable hosts.

B. Chlamydomonas reinhardtii Constituatively Expressing, Cyt1Aa.

A contemplated goal is to co-express Cyt1Aa with Cry11Aa in the chloroplast. Cyt1Aa has a strong synergistic effect on the Cry protoxins, especially Cry11Aa (Crickmore et al., 1995). Moreover, Cyt1Aa prevents the development of strong resistance in larval populations (Wirth et al., 1997), one of the great benefits of using Bti derived genes. Since Cyt1Aa is a small protein (27 kDa), the gene is also small.

At this time, the inventors have synthesized a codon-modified version of the cyt1Aa gene having an epitope tag at the C-terminus. This novel cyt1Aa gene was expressed in E. coli.

C. Chlamydomonas reinhardtii Constituatively Expressing Cry Genes Having a Starch-Binding Domain.

Additionally a codon-optimized starch-binding domain for ligation to Cry and/or Cryt genes of the present inventions is contemplated for ligating to the novel genes of the present inventions in order to reduce Cry protein damage to the host chloroplast. Therefore, an exemplary codon-modified starch-binding domain was designed and synthesized for use with genes of the present inventions.

In yet other embodiments, a codon-modified gene encoding a starch-binding domain is contemplated for use with Cry11A genes, individually or in combination with other cry and cryt genes.

The following references are herein incorporated by reference in their entirety:
1. Abdullah, et al., (2003). Introduction of Culex toxicity into Bacillus thuringiensis Cry4Ba by protein engineering. Applied and Environmental Microbiology 69, 5343-5353.
2. Al-yahyaee, S. A. S., and Ellar, D. J. (1995). Maximal toxicity of cloned CytA δ-endotoxin from Bacillus thuringiensis subsp. israelensis requires proteolytic processing from both the N- and C-termini. Microbiology 141, 3141-3148.
3. Amorim, L. B., de Oliveira, C. M. F., Rios, E. M., Regis, L., and Silva-Filha, M. H. N. L. (2007). Development of Culex quinquefasciatus resistance to Bacillus sphaericus strain IAB59 needs long term selection pressure. Biological Control 42, 155-160.
4. Angsuthanasombat, C., and Panyim, S. (1989). Biosynthesis of 130-kilodalton mosquito larvicide in the cyanobacterium Agmenellum quadruplicatum PR-6. Applied and Environmental Microbiology 55, 2428-2430.
5. Angsuthanasombat, C., Crickmore, N., and Ellar, D. J. (1992). Comparison of Bacillus thuringiensis subsp. israelensis CryIVA and CryIVB cloned toxins reveals synergism in vivo. FEMS Microbiology Letters 94, 63-68.
6. Angsuthanasombat, et al., (2004). Bacillus thuringiensis Cry4A and Cry4B mosquito-larvicidal proteins: homology-based 3D model and implications for toxin activity. Journal of Biochemistry and Molecular Biology 37, 304-313.
7. Aïssaoui, L. and Boudjelida, H. (2014). Larvicidal activity and influence of Bacillus thuringiensis (Vectobac G), on longevity and fecundity of mosquito species, European Journal of Experimental Biology 4, 104-109
8. Arapinis, C., de la Torre, F., and Szulmajster, J. (1988). Nucleotide and deduced amino acid sequence of the Bacillus sphaericus 1593M gene encoding a 51.4 kD polypeptide which acts synergistically with the 42 kD protein for expression of the larvicidal toxin. Nucleic Acids Research 16, 7731.
9. Arnon, D. I. (1949). Copper enzymes in isolated chloroplasts. Polyphenoloxidase in Beta vulgaris. Plant Physiology 24, 1-15.
10. Barloy, F., Delécluse, A., Nicolas, L., and Lecadet, M. M. (1996). Cloning and expression of the first anaerobic toxin gene from Clostridium bifermentans subsp. malaysia, encoding a new mosquitocidal protein with homologies to Bacillus thuringiensis delta-endotoxins. Journal of Bacteriology 178, 3099-3105.
11. Barnes, et al., (2005). Contribution of 5′- and 3′-untranslated regions of plastid mRNAs to the expression of Chlamydomonas reinhardtii chloroplast genes. Molecular Genetics and Genomics 274, 625-636.
12. Barton, K. A., Whiteley, H. R., and Yang, N.-S. (1987). Bacillus thuringiensis §-endotoxin expressed in transgenic Nicotiana tabacum provides resistance to lepidopteran insects. Plant Physiology 85, 1103-1109.
13. Baumann, P., Clark, M. A., Baumann, L., and Broadwell, A. H. (1991). Bacillus sphaericus as a mosquito pathogen: properties of the organism and its toxins. Microbiological Reviews 55, 425-436.
14. Becker, N., and Margalit, J. (1993). Use of Bacillus thuringiensis israelensis against mosquitoes and blackflies. In: Bacillus thuringiensis: an environmental biopesticide: theory and practice (Entwistle, P. F., Cory, J. S., Bailey, M. J., and Higgs, S., eds), 147-170, John Wiley & Sons, New York
15. Becker, N. (1997). Microbial control of mosquitoes: Management of the upper Rhine mosquito population as a model programme. Parasitology Today 13, 485-487.
16. Becker, N. (2000). Bacterial control of vector-mosquitoes and black flies. In: Entomopathogenic bacteria: from laboratory to field application (Charles, J. F., Delécluse, A., and Nielsen-LeRoux, C., eds), 383-398, Kluwer, Dordrecht, The Netherlands.
17. Becker, N. (2006). Microbial control of mosquitoes: Management of the Upper Rhine mosquito population as a model programme. In: An Ecological and Societal Approach to Biological Control 2 (Eilenberg, J. and Hokkanen, H. M. T., eds), 227-245, Springer Verlag, Berlin.
18. Ben-Dov, E. (2014). Bacillus thuringiensis subsp. israelensis and its dipteran-specific toxins. Toxins 6, 1222-1243.
19. Bernaudat, F., Frelet-Barrand, A., Pochon, N., Dementin, S., Hivin, P., Boutigny, S., Rioux, J.-B., Salvi, D., Seigneurin-Berny, D., Richaud, P., Joyard, J., Pignol, D., Sabaty, M., Desnos, T., Pebay-Peyroula, E., Darrouzet, E., Vernet, T., and Rolland, N. (2011). Heterologous expression of membrane proteins: choosing the appropriate host. PLoS ONE 6, e29191.
20. Berry, C., Hindley, J., Ehrhardt, A. F., Grounds, T., de Souza, I., and Davidson, E. W. (1993). Genetic determinants of host ranges of Bacillus sphaericus mosquito larvicidal toxins. Journal of Bacteriology 175, 510-518.
21. Berry, C., O'Neil, S., Ben-Dov, E., Jones, A. F., Murphy, L., Quail, M. A., Holden, M. T. G., Harris, D., Zaritsky, A., and Parkhill, J. (2002). Complete sequence and organization of pBtoxis, the toxin-coding plasmid of Bacillus thuringiensis subsp. israelensis. Applied and Environmental Microbiology 68, 5082-5095.
22. Boonserm, P., Pornwiroon, W., Katzenmeier, G., Panyim, S., and Angsuthanasombat, C. (2004). Optimised expression in Escherichia coli and purification of the functional form of the Bacillus thuringiensis Cry4Aa δ-endotoxin. Protein Expression and Purification 35, 397-403.
23. Boonserm, P., Davis, P., Ellar, D. J., and Li, J. (2005). Crystal Structure of the Mosquito-larvicidal toxin Cry4Ba and Its biological implications. Journal of Molecular Biology 348, 363-382.
24. Boonserm, P., Mo, M., Angsuthanasombat, C., and Lescar, J. (2006). Structure of the functional form of the mosquito larvicidal Cry4Aa toxin from Bacillus thuringiensis at a 2.8-Angstrom resolution. Journal of Bacteriology 188, 3391-3401.
25. Borovsky, D., Khasdan, V., Nauwelaers, S., Theunis, C., Bertie0r, L., Ben-Dov, E., and Zaritsky, A. (2010). Synergy between Aedes aegypti trypsin modulating oostatic factor and δ-Endotoxins. The Open Toxinology Journal 3, 141-150.
26. Borovsky, D., Naywelaers, S., Mileghem, A., Meyvis, Y., Laeremans, A., Theunis, C., Bertie, L., and Boons, E. (2011). Control of mosquito larvae with TMOF and 60 kDa Cry4Aa expressed in Pichia pastoris. Pestycydy/Pesticides 1-4, 5-15.
27. Boussiba, S., Wu, X. Q., Ben-Dov, E., Zarka, A., and Zaritsky, A. (2000). Nitrogen-fixing cyanobacteria as gene delivery system for expressing mosquitocidal toxins of Bacillus thuringiensis ssp. israelensis. Journal of Applied Phycology 12, 461-467.
28. Boynton, J. E., Gillham, N. W., Harris, E., Hosler, J., Johnson, A., Jones, A., Randolph-Anderson, B., Robertson, D., Klein, T., Shark, K., and et, al. (1988). Chloroplast transformation in Chlamydomonas with high velocity microprojectiles. Science 240, 1534-1538.
29. Boynton, J. E., and Gillham, N. W. (1993). Chloroplast transformation in Chlamydomonas. In: Methods for transforming animal and plant cells, Methods in enzymology 217 (Ray, W., ed), 510-536, Academic Press, New York.
30. Bradley, B. A., and Quarmby, L. M. (2005). A NIMA-related kinase, Cnk2p, regulates both flagellar length and cell size in Chlamydomonas. Journal of Cell Science 118, 3317-3326.
31. Bravo, A., Gill, S. S., and Soberón, M. (2007). Mode of action of Bacillus thuringiensis Cry and Cyt toxins and their potential for insect control. Toxicon 49, 423-435.
32. Bravo, A., Likitvivatanavong, S., Gill, S. S., and Soberón, M. (2011). Bacillus thuringiensis: A story of a successful bioinsecticide. Insect Biochemistry and Molecular Biology 41, 423-431.
33. Bukhari, D., and Shakoori, A. (2009). Cloning and expression of Bacillus thuringiensis cry11 crystal protein gene in Escherichia coli. Mol Biol Rep 36, 1661-1670.
34. Butko, P., Huang, F., Pusztai-Carey, M., and Surewicz, W. K. (1996). Membrane permeabilization induced by cytolytic δ-endotoxin CytA from Bacillus thuringiensis var. israelensis. Biochemistry 35, 11355-11360.
35. Butko, P. (2003). Cytolytic toxin Cyt1a and its mechanism of membrane damage: data and hypotheses. Applied and Environmental Microbiology 69, 2415-2422.
36. Cahan, R., Friman, H., and Nitzan, Y. (2008). Antibacterial activity of Cyt1Aa from Bacillus thuringiensis subsp. israelensis. Microbiology 154, 3529-3536.
37. Campos-Quevedo, N., Rosales-Mendoza, S., Paz-Maldonado, L., Martinez-Salgado, L., Guevara-Arauza, J., and Soria-Guerra, R. (2013). Production of milk-derived bioactive peptides as precursor chimeric proteins in chloroplasts of Chlamydomonas reinhardtii. Plant Cell, Tissue and Organ Culture 113, 217-225.
38. Canan, U. (2013). Microorganisms in Biological Pest Control—A Review (Bacterial Toxin Application and Effect of Environmental Factors). In: Current Progress in Biological Research, (Silva-Opps M, ed), InTech, DOI: 10.5772/55786. Available from: www.intechopen.com/books/current-progress-in-biological-research/microorganisms-in-biological-pest-control-a-review-bacterial-toxin-application-and-effect-of-environ.
39. CDC (2014, Jun. 2). West Nile Virus: final annual maps & data for 1999-2013 (GA, USA). Available from www.cdc.gov/westnile/statsMaps/finalMapsData/index.html.
40. Cerutti, H., Johnson, A. M., Gillham, N. W., and Boynton, J. E. (1997). Epigenetic silencing of a foreign gene in nuclear transformants of Chlamydomonas. The Plant Cell 9, 925-945.
41. Chakrabarti, S., Lutz, K., Lertwiriyawong, B., Svab, Z., and Maliga, P. (2006). Expression of the cry9Aa2 B.t. gene in tobacco chloroplasts confers resistance to potato tuber moth. Transgenic Research 15, 481-488.
42. Chapman, H. C. (1974). Biological control of mosquito larvae. Annual Review of Entomology 19, 33-59.
43. Chen, H.-C., and Melis, A. (2013). Marker-free genetic engineering of the chloroplast in the green microalga Chlamydomonas reinhardtii. Plant Biotechnology Journal 11, 818-828.
44. Chen, J., Aimanova, K. G., Fernandez, L. E., Bravo, A., Soberon, M., and Gill, S. S. (2009). Aedes aegypti cadherin serves as a putative receptor of the Cry11Aa toxin from Bacillus thuringiensis subsp. israelensis. Biochemical Journal 424, 191-200.
45. Chen, J., Likitvivatanavong, S., Aimanova, K. G., and Gill, S. S. (2013). A 104 kDa Aedes aegypti aminopeptidase N is a putative receptor for the Cry11Aa toxin from Bacillus thuringiensis subsp. israelensis. Insect Biochemistry and Molecular Biology 43, 1201-1208.
46. Chen, S., Kaufman, M. G., Korir, M. L., and Walker, E. D. (2014). Ingestibility, Digestibility, and engineered biological control potential of Flavobacterium hibernum, isolated from larval mosquito habitats. Applied and Environmental Microbiology 80, 1150-1158.
47. Chilcott, C. N., and Ellar, D. J. (1988). Comparative toxicity of Bacillus thuringiensis var. israelensis crystal proteins in vivo and in vitro. Journal of General Microbiology 134, 2551-2558.
48. Chilcott, C. N., Knowles, B. H., Ellar, D. J., Drobniewski, F. A. (1990). Mechanism of action of Bacillus thuringiensis israelensis parasporal body. In: Bacterial Control of Mosquitoes and black flies: biochemistry, genetics, & applications of Bacillus thuringiensis israelensis and Bacillus sphaericus (De Barjac, H., and Sutherland, D., eds), 202-217, Rutgers University Press, New Brunswick, N.J.
49. Choquet, Y., and Wollman, F.-A. (2002). Translational regulations as specific traits of chloroplast gene expression. FEBS Letters 529, 39-42.
50. Chungjatupornchai, W., HÖFte, H., Seurinck, J., Angsuthanasombat, C., and Vaeck, M. (1988). Common features of Bacillus thuringiensis toxins specific for Diptera and Lepidoptera. European Journal of Biochemistry 173, 9-16.
51. Cohen, S., Albeck, S., Ben-Dov, E., Cahan, R., Firer, M., Zaritsky, A., and Dym, O. (2011). Cyt1Aa toxin: crystal structure reveals implications for its membrane-perforating function. Journal of Molecular Biology 413, 804-814.
52. Coragliotti, A., Beligni, M., Franklin, S., and Mayfield, S. (2011). Molecular factors affecting the accumulation of recombinant proteins in the Chlamydomonas reinhardtii chloroplast. Molecular Biotechnology 48, 60-75.
53. Crickmore, N., Bone, E. J., Williams, J. A., and Ellar, D. J. (1995). Contribution of the individual components of the δ-endotoxin crystal to the mosquitocidal activity of Bacillus thuringiensis subsp. israelensis. FEMS Microbiology Letters 131, 249-254.
54. Crickmore, N., Zeigler, D. R., Feitelson, J., Schnepf, E., Van Rie, J., Lereclus, D., Baum, J., and Dean, D. H. (1998). Revision of the nomenclature for the Bacillus thuringiensis pesticidal crystal proteins. Microbiology and Molecular Biology Reviews 62, 807-813.
55. Dai, S.-M., and Gill, S. S. (1993). In vitro and in vivo proteolysis of the Bacillus thuringiensis subsp. israelensis CryIVD protein by Culex quinquefasciatus larval midgut proteases. Insect Biochemistry and Molecular Biology 23, 273-283.
56. Darboux, I., Nielsen-LeRoux, C., Charles, J., and Pauron, D. (2001). The receptor of Bacillus sphaericus binary toxin in Culex pipiens (Diptera: Culicidae) midgut: molecular cloning and expression. Insect Biochemistry and Molecular Biology 31, 981-990.
57. de Barjac, H. (1978). A new subspecies of Bacillus thuringiensis very toxic for mosquitoes; Bacillus thuringiensis serotype H-14. Compte Rendu Académie Sciences Paris Series D. 286, 797-800.
58. de Barjac, H. (1989). New facts and trends in bacteriological control of mosquitoes. Memórias do Instituto Oswaldo Cruz 84, 101-105.
59. De Cosa, B., Moar, W., Lee, S.-B., Miller, M., and Daniell, H. (2001). Overexpression of the Bt cry2Aa2 operon in chloroplasts leads to formation of insecticidal crystals. Nature Biotechnology 19, 71-74.
60. de Maagd, R. A., Bravo, A., and Crickmore, N. (2001). How Bacillus thuringiensis has evolved specific toxins to colonize the insect world. Trends in Genetics 17, 193-199.
61. Delécluse, A., Poncet, S., Klier, A., and Rapoport, G. (1993). Expression of cryIVA and cryIVB genes, independently or in combination, in a crystal-negative strain of Bacillus thuringiensis subsp. israelensis. Applied and Environmental Microbiology 59, 3922-3927.
62. Delécluse, A., Rosso, M. L., and Ragni, A. (1995). Cloning and expression of a novel toxin gene from Bacillus thuringiensis subsp. jegathesan encoding a highly mosquitocidal protein. Applied and Environmental Microbiology 61, 4230-4235.
63. Delécluse, A., Juárez-Pérez, V., and Berry, C. (2000). Vector-active toxins: structure and diversity. In: Entomopathogenic Bacteria: from Laboratory to Field Application (Charles, J. F., Delécluse, A., and Roux C. N., eds), 101-125, Springer Science & Business Media, The Netherland.
64. Deng, C., Peng, Q., Song, F., and Lereclus, D. (2014). Regulation of cry gene expression in Bacillus thuringiensis. Toxins 6, 2194-2209.
65. Dervyn, E., Poncet, S., Klier, A., and Rapoport, G. (1995). Transcriptional regulation of the cryIVD gene operon from Bacillus thuringiensis subsp. israelensis. Journal of Bacteriology 177, 2283-2291.
66. Drapier, et al., (1998). The chloroplast atpa gene cluster in Chlamydomonas reinhardtii. Plant Physiology 117, 629-641.
67. Eberhard, S., Drapier, D., and Wollman, F. A. (2002). Searching limiting steps in the expression of chloroplast-encoded proteins: relations between gene copy number, transcription, transcript abundance and translation rate in the chloroplast of Chlamydomonas reinhardtii. The Plant Journal 31, 149-160.
68. Economou, C., Wannathong, T., Szaub, J., and Purton, S. (2014). A simple, low-cost method for chloroplast transformation of the green alga Chlamydomonas reinhardtii. In: Chloroplast Biotechnology, Methods in Molecular Biology 1132 (Maliga, P., ed), 401-411, Humana Press, New York,
69. Einhauer, A., and Jungbauer, A. (2001). The FLAG™ peptide, a versatile fusion tag for the purification of recombinant proteins. Journal of Biochemical and Biophysical Methods 49, 455-465.
70. El-Bendary, M. A. (2006). Bacillus thuringiensis and Bacillus sphaericus biopesticides production. Journal of Basic Microbiology 46, 158-170.
71. Erickson, et al., (1986). Lack of the D2 protein in a Chlamydomonas reinhardtii psbD mutant affects photosystem II stability and D1 expression. The EMBO Journal 5, 1745-1754.
72. Fansiri, et al., (2006). Laboratory and semi-field evaluation of mosquito dunks against Aedes aegypti and Aedes albopictus larvae (Diptera: Culicidae). The Southeast Asian Journal of Tropical Medicine and Public Health 37, 62-66.
73. Fargo, D. C., Zhang, M., Gillham, N. W., and Boynton, J. E. (1998). Shine-Dalgarno-like sequences are not required for translation of chloroplast mRNAs in Chlamydomonas reinhardtii chloroplasts or in Escherichia coli. Molecular Genetics and Genomics 257, 271-282.
74. Federici, B. A., Park, H.-W., Bideshi, D. K., Wirth, M. C., and Johnson, J. J. (2003). Recombinant bacteria for mosquito control. Journal of Experimental Biology 206, 3877-3885.
75. Fernandez, et al., (2005). Cry11Aa toxin from Bacillus thuringiensis binds its receptor in Aedes aegypti mosquito larvae through loop α-8 of domain II. FEBS Letters 579, 3508-3514.
76. Fernandez, L. E., Aimanova, K. G., Gill, S. S., Bravo, A., and Soberón, M. (2006). A GPI-anchored alkaline phosphatase is a functional midgut receptor of Cry11Aa toxin in Aedes aegypti larvae. Biochemical Journal 394, 77-84.
77. Fernandez-Luna, M. T., Lanz-Mendoza, H., Gill, S. S., Bravo, A., Soberon, M., and Miranda-Rios, J. (2010). An α-amylase is a novel receptor for Bacillus thuringiensis ssp. israelensis Cry4Ba and Cry11Aa toxins in the malaria vector mosquito Anopheles albimanus (Diptera: Culicidae). Environmental Microbiology 12, 746-757.
78. Ferris, P. J., Pavlovic, C., Fabry, S., and Goodenough, U. W. (1997). Rapid evolution of sex-related genes in Chlamydomonas. Proceedings of the National Academy of Sciences of the United States of America 94, 8634-8639.
79. Finney, D. J. (1977). Probit analysis, Cambridge University Press, Cambridge, U. K.
80. Fischer, N., Stampacchia, O., Redding, K., and Rochaix, J.-D. (1996). Selectable marker recycling in the chloroplast. Molecular Genetics and Genomics 251, 373-380.
81. Fischer, R., Stoger, E., Schillberg, S., Christou, P., and Twyman, R. M. (2004). Plant-based production of biopharmaceuticals. Current Opinion in Plant Biology 7, 152-158.
82. Fischhoff, D. A., Bowdish, K. S., Perlak, F. J., Marrone, P. G., McCormick, S. M., Niedermeyer, J. G., Dean, D. A., Kusano-Kretzmer, K., Mayer, E. J., Rochester, D. E., Rogers, S. G., and Fraley, R. T. (1987). Insect tolerant transgenic tomato plants. Nature Biotechnology 5, 807-813.
83. Fletcher, S., Muto, M., and Mayfield, S. (2007). Optimization of recombinant protein expression in the chloroplasts of green algae. In: Transgenic microalgae as green cell factories, Advances in Experimental Medicine and Biology 616 (Leon, R., Galván, A., and Fernández, E., eds), 90-98, Springer, New York,
84. Frankenhuyzen, K. V. (2009). Insecticidal activity of Bacillus thuringiensis crystal proteins. Journal of Invertebrate Pathology 101, 1-16.
85. Franklin, S., Ngo, B., Efuet, E., and Mayfield, S. P. (2002). Development of a GFP reporter gene for Chlamydomonas reinhardtii chloroplast. The Plant Journal 30, 733-744.
86. Gazit, E., Rocca, P. L., Sansom, M. S. P., and Shai, Y. (1998). The structure and organization within the membrane of the helices composing the pore-forming domain of Bacillus thuringiensis δ-endotoxin are consistent with an “umbrella-like” structure of the pore. Proceedings of the National Academy of Sciences of the United States of America 95, 12289-12294.
87. Geisel, N. (2011). Constitutive versus responsive gene expression strategies for growth in changing environments. PLoS ONE 6, e27033.
88. Glare, T. R., and O'Callaghan, M. (1998). Environmental and health impacts of Bacillus thuringiensis israelensis, Report for The Ministry of Health.
89. Glare, T. R. and O'Callaghan, M. (2000) Bacillus thuringiensis: Biology, Ecology and Safety. J Wiley & Sons, West Sussex UK
90. Goldberg, L. J., and Margalit, J. (1977). A bacterial spore demonstrating rapid larvicidal activity against Anopheles sergentii, Uranotaenia unguiculata, Culex univittatus, Aedes aegypti and Culex pipiens. Mosquito News 37, 355-358.
91. Goldschmidt-Clermont, M. (1991). Transgenic expression of aminoglycoside adenine transferase in the chloroplast: a selectable marker for site-directed transformation of Chlamydomonas. Nucleic Acids Research 19, 4083-4089.
92. Gorman, D. S., and Levine, R. P. (1965). Cytochrome f and plastocyanin: their sequence in the photosynthetic electron transport chain of Chlamydomonas reinhardi. Proceedings of the National Academy of Sciences of the United States of America 54, 1665-1669.
93. Gorton, H. L., and Vogelmann, T. C. (2003). Ultraviolet radiation and the snow alga Chlamydomonas nivalis (Bauer) Wille. Photochemistry and Photobiology 77, 608-615.
94. Grant, D. M., Gillham, N. W., and Boynton, J. E. (1980). Inheritance of chloroplast DNA in Chlamydomonas reinhardtii. Proceedings of the National Academy of Sciences of the United States of America 77, 6067-6071.
95. Grisolia, et al., (2009). Acute toxicity and cytotoxicity of Bacillus thuringiensis and Bacillus sphaericus strains on fish and mouse bone marrow. Ecotoxicology 18, 22-26.
96. Gullet, P., Kurtak, D. C., Philippon, B., and Meyer, R. (1990) Use of Bacillus thuringiensis israelensis for Onchocerciasis control in west Africa, In: Bacterial Control of Mosquitoes and black flies: biochemistry, genetics, & applications of Bacillus thuringiensis israelensis and Bacillus sphaericus (De Barjac, H., and Sutherland, D., eds), 187-199, Rutgers University Press, New Brunswick, N.J.
97. Gustafsson, C., Govindarajan, S., and Minshull, J. (2004). Codon bias and heterologous protein expression. Trends in Biotechnology 22, 346-353.
98. Halford, N. G. (2012). Toward two decades of plant biotechnology: successes, failures, and prospects. Food and Energy Security 1, 9-28.
99. Harris, E. H. (1989). The Chlamydomonas Sourcebook: A Comprehensive Guide to Biology and Laboratory Use. Academic Press, San Diego.
100. Harris, E. H., Burkhart, B. D., Gillham, N. W., and Boynton, J. E. (1989). Antibiotic resistance mutations in the chloroplast 16S and 23S rRNA genes of Chlamydomonas reinhardtii: correlation of genetic and physical maps of the chloroplast genome. Genetics 123, 281-292.
101. Harris, E. H., Boynton, J. E., and Gillham, N. W. (1994). Chloroplast ribosomes and protein synthesis. Microbiological Reviews 58, 700-754.
102. Harris, E. H. (2001). Chlamydomonas as a model organism. Annual Review of Plant Physiology and Plant Molecular Biology 52, 363-406.
103. Harris, E. H. (2009). The Chlamydomonas Sourcebook, Volume 1: Introduction to Chlamydomonas and its laboratory use, 2^{nd} edition, Academic Press, San Diego
104. Hayakawa, T., Howlader, M., Yamagiwa, M., and Sakai, H. (2008). Design and construction of a synthetic Bacillus thuringiensis Cry4Aa gene: Hyperexpression in Escherichia coli. Applied Microbiology and Biotechnology 80, 1033-1037.
105. Herrin, D., Michaels, A., and Hickey, E. (1981). Synthesis of a chloroplast membrane polypeptide on thylakoid-bound ribosomes during the cell cycle of Chlamydomonas reinhardii 137+. Biochimica et Biophysica Acta (BBA)—Nucleic Acids and Protein Synthesis 655, 136-145.
106. Herrin, D., and Schmidt, G. (1987). Chloroplast gene expression in chloroplast ribosome-deficient mutants of Chlamydomonas Reinhardtii. In: Progress in Photosynthesis Research (Biggins, J., ed), 645-648, Martinus Nijhoff, Dordrecht.
107. Herrin, D., and Nickelsen, J. (2004). Chloroplast RNA processing and stability. Photosynthesis Research 82, 301-314.
108. Hate, H., and Whiteley, H. R. (1989). Insecticidal crystal proteins of Bacillus thuringiensis. Microbiological Reviews 53, 242-255.
109. Holzinger, A., and Lutz, C. (2006). Algae and UV irradiation: Effects on ultrastructure and related metabolic functions. Micron 37, 190-207.
110. Horák, P., Weiser, J., Mikeš, L., and Kolářovà, L. (1996). The effect of Bacillus thuringiensis M-exotoxin on Trematode Cercariae. Journal of Invertebrate Pathology 68, 41-49.
111. Howlader, M. T. H., Kagawa, Y., Sakai, H., and Hayakawa, T. (2009). Biological properties of loop-replaced mutants of Bacillus thuringiensis mosquitocidal Cry4Aa. Journal of Bioscience and Bioengineering 108, 179-183.
112. Hsieh, et al., (2013). The proteome of copper, iron, zinc, and manganese micronutrient deficiency in Chlamydomonas reinhardtii. Molecular & Cellular Proteomics 12, 65-86.
113. Hughes, P. (2005). Bloodworm resistant rice, Rice CRC final report. Cooperative Research Centre for Sustainable Rice.
114. Ibarra, J. E., and Federici, B. A. (1986). Isolation of a relatively nontoxic 65-kilodalton protein inclusion from the parasporal body of Bacillus thuringiensis subsp. israelensis. Journal of Bacteriology 165, 527-533.
115. Ishikura, et al., (1999). Expression of a foreign gene in Chlamydomonas reinhardtii chloroplast. Journal of Bioscience and Bioengineering 87, 307-314.
116. Ji, Q., Vincken, J.-P., Suurs, L. J. M., and Visser, R. F. (2003). Microbial starch-binding domains as a tool for targeting proteins to granules during starch biosynthesis. Plant Molecular Biology 51, 789-801.
117. Juntadech, et al., (2012). Efficient transcription of the larvicidal cry4Ba gene from Bacillus thuringiensis in transgenic chloroplasts of the green algal Chlamydomonas reinhardtii. Advances in Bioscience and Biotechnology 3, 362-369.
118. Jurat-Fuentes, J. L., and Jackson, T. A. (2012). Bacterial Entomopathogens. In: Insect Pathology, 2^{nd} edition (Vega, F. E. and Kaya, H. K., eds), 265-349, Academic Press, San Diego.
119. Kasai, et al., (2003). Effect of coding regions on chloroplast gene expression in Chlamydomonas reinhardtii. Journal of Bioscience and Bioengineering 95, 276-282.
120. Kaufman, et al., (2006). Importance of algal biomass to growth and development of Anopheles gambiae larvae. Journal of Medical Entomology 43, 669-676.
121. Kellen, W. R., Clark, T. B., Lindegren, J. E., and Ho, B. C. (1965). Bacillus sphaericus Neide as a pathogen of mosquitoes. Journal of Invertebrate Pathology 7, 442-448.
122. Khasdan, et al., (2003). Mosquito larvicidal activity of transgenic Anabaena PCC 7120 expressing toxin genes from Bacillus thuringiensis subsp. israelensis. FEMS Microbiology Letters 227, 189-195.
123. Kindle, K., and Sodeinde, O. (1994). Nuclear and chloroplast transformation in Chlamydomonas reinhardtii: strategies for genetic manipulation and gene expression. Journal of Applied Phycology 6, 231-238.
124. Kleter, et al., (2007). Altered pesticide use on transgenic crops and the associated general impact from an environmental perspective. Pest Management Science 63, 1107-1115.
125. Komano, T., Yamagiwa, M., Nishimoto, T., Yoshisue, H., Tanabe, K., Sen, K., and Sakai, H. (1998). Activation process of the insecticidal proteins CryIVA and CryIVB produced by Bacillus thuringiensis subsp. israelensis. Israel Journal of Entomology 32, 184-198.
126. Kuchka, M. R., Goldschmidt-Clermont, M., van Dillewijn, J., and Rochaix, J.-D. (1989). Mutation at the Chlamydomonas nuclear NAC2 locus specifically affects stability of the chloroplast psbD transcript encoding polypeptide D2 of PS II. Cell 58, 869-876.
127. Kumar, A. (2010). Optimization of transgene expression in Chlamydomonas reinhardtii and its biotechnological applications. Ph.D. dissertation. The Ohio State University.
128. Kumar, A., Wang, S., Ou, R., Samrakandi, M., Beerntsen, B. T., and Sayre, R. T. (2013). Development of an RNAi based microalgal larvicide to control mosquitoes. Malarial World Journal 4, 1-6.
129. Kwon, T., Odom, O., Qiu, W., and Herrin, D. (2014). PCR analysis of chloroplast double-strand break (dsb) repair products induced by I-CreII in Chlamydomonas and Arabidopsis. In: Homing Endonucleases, Methods in Molecular Biology 1123 (Edgell, D. R., ed), 77-86, Humana Press, New York.
130. Lacey, L., and Merritt, R. (2003). The safety of bacterial microbial agents used for black fly and mosquito control in aquatic environments. In: Environmental Impacts of Microbial Insecticides, Progress in Biological Control, 1 (Hokkanen, H. T. and Hajek, A., eds), 151-168, Kluwer Academic Press, The Netherlands.
131. Laemmli, U. K. (1970). Cleavage of Structural Proteins during the assembly of the head of bacteriophage T4. Nature 227, 680-685.
132. Lauersen, K. J., Berger, H., Mussgnug, J. H., and Kruse, O. (2013). Efficient recombinant protein production and secretion from nuclear transgenes in Chlamydomonas reinhardtii. Journal of Biotechnology 167, 101-110.
133. Laurence, D. s., Christophe, L., and Roger, F. (2011). Using the bio-insecticide Bacillus Thuringiensis israelensis in mosquito control. In Pesticides in the Modern World—Pests Control and Pesticides Exposure and Toxicity Assessment (Stoytcheva, D. M., ed), InTech, DOI: 10.5772/948. Available from: www.intechopen.com/books/pesticides-in-the-modern-world-pests-control-and-pesticides-exposure-and-toxicity-assessment.
134. Lee, J., and Herrin, D. L. (2002). Assessing the relative importance of light and the circadian clock in controlling chloroplast translation in Chlamydomonas reinhardtii. Photosynthesis Research 72, 295-306.
135. Leetachewa, S., Katzenmeier, G., and Angsuthanasombat, C. (2006). Novel preparation and characterization of the α4-loop-α5 membrane-perturbing peptide from the Bacillus thuringiensis Cry4Ba δ-endotoxin. Journal of biochemistry and molecular biology 39, 270-277.
136. Li, J., Carroll, J., and Ellar, D. J. (1991). Crystal structure of insecticidal δ-endotoxin from Bacillus thuringiensis at 2.5 A resolution. Nature 353, 815-821.
137. Li, J., Koni, P. A., and Ellar, D. J. (1996). Structure of the mosquitocidal δ-endotoxin CytB from Bacillus thuringiensis sp. kyushuensis and Implications for Membrane Pore Formation. Journal of Molecular Biology 257, 129-152.
138. Lister, D. L., Bateman, J. M., Purton, S., and Howe, C. J. (2003). DNA transfer from chloroplast to nucleus is much rarer in Chlamydomonas than in tobacco. Gene 316, 33-38.
139. Liu, Y.-T., Sui, M.-J., Ji, D.-D., Wu, I. H., Chou, C.-C., and Chen, C.-C. (1993). Protection from ultraviolet irradiation by melanin of mosquitocidal activity of Bacillus thuringiensis var. israelensis. Journal of Invertebrate Pathology 62, 131-136.
140. Lössl, A., Eibl, C., Harloff, H. J., Jung, C., and Koop, H. U. (2003). Polyester synthesis in transplastomic tobacco (Nicotiana tabacum L.): significant contents of polyhydroxybutyrate are associated with growth reduction. Plant Cell Reports 21, 891-899.
141. Magee, et al., (2004). T7 RNA polymerase-directed expression of an antibody fragment transgene in plastids causes a semi-lethal pale-green seedling phenotype. Transgenic Research 13, 325-337.
142. Manasherob, et al., (2001). Effect of Accessory Proteins P19 and P20 on Cytolytic Activity of Cyt1Aa from Bacillus thuringiensis subsp. israelensis in Escherichia coli. Current Microbiology 43, 355-364.
143. Manasherob, et al., (2002). Protection from UV-B damage of mosquito larvicidal toxins from Bacillus thuringiensis subsp. israelensis expressed in Anabaena PCC 7120. Current Microbiology 45, 217-220.
144. Manasherob, R., Zaritsky, A., Metzler, Y., Ben-Dov, E., Itsko, M., and Fishov, I. (2003). Compaction of the Escherichia coli nucleoid caused by Cyt1Aa. Microbiology 149, 3553-3564.
145. Manceva, S. D., Pusztai-Carey, M., Russo, P. S., and Butko, P. (2005). A detergent-like mechanism of action of the cytolytic toxin Cyt1A from Bacillus thuringiensis var. israelensis. Biochemistry 44, 589-597.
146. Manuell, A., and Mayfield, S. (2006). A bright future for Chlamydomonas. Genome Biology 7, 327.321-327.323.
147. Manuell, A. L., Beligni, M. V., Elder, J. H., Siefker, D. T., Tran, M., Weber, A., McDonald, T. L., and Mayfield, S. P. (2007). Robust expression of a bioactive mammalian protein in Chlamydomonas chloroplast. Plant Biotechnology Journal 5, 402-412.
148. Margalit, Y. (1989). Biological control by Bacillus thuringiensis subsp. israelensis (Bti); history and present status. Israel Journal of Entomology 23, 3-8.
149. Margalith, Y., and Ben-Dov, E. (1999). Biological control by Bacillus thuringiensis subsp. israelensis. In Insect Pest Management: Techniques for Environmental Protection (Rechcigl, J. E. and Rechcigl, N. A., eds), CRC Press, 244-303.
150. Marin-Navarro, J., Manuell, A., Wu, J., and P. Mayfield, S. (2007). Chloroplast translation regulation. Photosynthesis Research 94, 359-374.
151. Marten, G. G. (1986). Mosquito control by plankton management: the potential of indigestible green algae. The Journal of tropical medicine and hygiene 89, 213-222.
152. Matsuo, T., Onai, K., Okamoto, K., Minagawa, J., and Ishiura, M. (2006). Real-time monitoring of chloroplast gene expression by a luciferase reporter: evidence for nuclear regulation of chloroplast circadian period. Molecular and Cellular Biology 26, 863-870.
153. Maul, et al., (2002). The Chlamydomonas reinhardtii plastid chromosome: islands of genes in a sea of repeats. The Plant Cell 14, 2659-2679.
154. Mayfield, S. P., Franklin, S. E., and Lerner, R. A. (2003). Expression and assembly of a fully active antibody in algae. Proceedings of the National Academy of Sciences of the United States of America 100, 438-442.
155. Mayfield, S. P., and Schultz, J. (2004). Development of a luciferase reporter gene, luxCt, for Chlamydomonas reinhardtii chloroplast. The Plant Journal 37, 449-458.
156. Memon, A. R., Herrin, D. L., and Thompson Jr, G. A. (1993). Intracellular translocation of a 28 kDa GTP-binding protein during osmotic shock-induced cell volume regulation in Dunaliella salina. Biochimica et Biophysica Acta (BBA)—Molecular Cell Research 1179, 11-22.
157. Merchant, et al., (2007). The Chlamydomonas genome reveals the evolution of key animal and plant functions. Science 318, 245-250.
158. Michelet, L., Lefebvre-Legendre, L., Burr, S. E., Rochaix, J.-D., and Goldschmidt-Clermont, M. (2011). Enhanced chloroplast transgene expression in a nuclear mutant of Chlamydomonas. Plant Biotechnology Journal 9, 565-574.
159. Milam, C. D., Farris, J. L., and Wilhide, J. D. (2000). Evaluating mosquito control pesticides for effect on target and nontarget organisms. Archives of Environmental Contamination and Toxicology 39, 324-328.
160. Minai, L., Wostrikoff, K., Wollman, F.-A., and Choquet, Y. (2006). Chloroplast biogenesis of photosystem II cores involves a series of assembly-controlled steps that regulate translation. The Plant Cell 18, 159-175.
161. Minko, et al., (1999). Renilla luciferase as a vital reporter for chloroplast gene expression in Chlamydomonas. Molecular Genetics and Genomics 262, 421-425.
162. Misquitta, R. and Herrin, D. L. (2005). Circadian Regulation of Chloroplast Transcription: A Review. Plant Tissue Culture 15, 83-101
163. Miyamoto, J., Kaneko, H., Tsuji, R., and Okuno, Y. (1995). Pyrethroids, nerve poisons: how their risks to human health should be assessed. Toxicology Letters 82-83, 933-940.
164. Murray, et al., (2002). Expression of biotin-binding proteins, avidin and streptavidin, in plant tissues using plant vacuolar targeting sequences. Transgenic Research 11, 199-214.
165. Myasnik, et al., (2001). Comparative sensitivity to UV-B radiation of two Bacillus thuringiensis subspecies and other Bacillus sp. Current Microbiology 43, 140-143.
166. Nakamura, Y., Gojobori, T., and Ikemura, T. (2000). Codon usage tabulated from international DNA sequence databases: status for the year 2000. Nucleic Acids Research 28, 292.
167. Nauen, R. (2007). Insecticide resistance in disease vectors of public health importance. Pest Management Science 63, 628-633.
168. Newman, et al., (1990). Transformation of chloroplast ribosomal RNA genes in Chlamydomonas: molecular and genetic characterization of integration events. Genetics 126, 875-888.
169. Newman, S. M., Harris, E. H., Johnson, A. M., Boynton, J. E., and Gillham, N. W. (1992). Nonrandom distribution of chloroplast recombination events in Chlamydomonas reinhardtii: evidence for a hotspot and an adjacent cold region. Genetics 132, 413-429.
170. Nickelsen, et al., (1999). Identification of cis-acting rna leader elements required for chloroplast psbD gene expression in Chlamydomonas. The Plant Cell 11, 957-970.
171. Nicolas, L., Dossou-Yovo, J., and Hougard, J.-M. (1987). Persistence and recycling of Bacillus sphaericus 2362 spores in Culex quinquefasciatus breeding sites in west africa. Applied Microbiology and Biotechnology 25, 341-345.
172. Nicolas, L., Nielsen-Leroux, C., Charles, J.-F., and Delécluse, A. (1993). Respective role of the 42- and 51-kDa components of the Bacillus sphaericus toxin overexpressed in Bacillus thuringiensis. FEMS Microbiology Letters 106, 275-279.
173. Nielsen-Leroux, C., Charles, J.-F., Thiéry, I., and Georghiou, G. P. (1995). Resistance in a laboratory population of Culex Quinquefasciatus (Diptera: Culicidae) to Bacillus Sphaericus binary toxin is due to a change in the receptor on midgut brush-border membranes. European Journal of Biochemistry 228, 206-210.
174. Noth, J., Krawietz, D., Hemschemeier, A., and Happe, T. (2013). Pyruvate: ferredoxin oxidoreductase is coupled to light-independent hydrogen production in Chlamydomonas reinhardtii. Journal of Biological Chemistry 288, 4368-4377.
175. Odom, O. W., Holloway, S. P., Deshpande, N. N., Lee, J., and Herrin, D. L. (2001). Mobile self-splicing group I introns from the psbA gene of Chlamydomonas reinhardtii: highly efficient homing of an exogenous intron containing its own promoter. Molecular and Cellular Biology 21, 3472-3481.
176. Odom, O. W., and Herrin, D. L. (2013). Reverse transcription of spliced psbA mRNA in Chlamydomonas spp. and its possible role in evolutionary intron loss. Molecular Biology and Evolution 30, 2666-2675.
177. Oey, M., Lohse, M., Kreikemeyer, B., and Bock, R. (2009). Exhaustion of the chloroplast protein synthesis capacity by massive expression of a highly stable protein antibiotic. The Plant Journal 57, 436-445.
178. Ohana, B., Margalit, J., and Barak, Z. E. (1987). Fate of Bacillus thuringiensis subsp. israelensis under simulated field conditions. Applied and Environmental Microbiology 53, 828-831.
179. Oliveira-Filho, et al., (2014). evaluating the elimination of brazilian entomopathogenic Bacillus by non-target aquatic species: an experimental study. Bulletin of Environmental Contamination and Toxicology 93, 461-464.
180. Orduz, S., Realpe, M., Arango, R., Murillo, L. A., and Dele\ddot{}cluse, A. (1998). Sequence of the cry11Bb11 gene from Bacillus thuringiensis subsp. medellin and toxicity analysis of its encoded protein. Biochimica et Biophysica Acta 1388, 267-272
181. Otieno-Ayayo, et al., (2008). Variations in the mosquito larvicidal activities of toxins from Bacillus thuringiensis ssp. israelensis. Environmental Microbiology 10, 2191-2199.
182. Pantuwatana, S., Maneeroj, R., and Upatham, E. (1989). Long residual activity of Bacillus sphaericus 1593 against Culex quinquefasciatus larvae in artificial pools. Southeast Asian Journal of Tropical Medicine and Public Health 20, 421-427.
183. Park, H.-W., Bideshi, D. K., and Federici, B. A. (2000). Molecular genetic manipulation of truncated Cry1C Protein Synthesis in Bacillus thuringiensis to improve stability and yield. Applied and Environmental Microbiology 66, 4449-4455.
184. Park, et al., (2005). Recombinant larvicidal bacteria with markedly improved efficacy against Culex vectors of west nile virus. The American Journal of Tropical Medicine and Hygiene 72, 732-738.
185. Pena-Montenegro, T. D., and Dussán, J. (2013). Genome sequence and description of the heavy metal tolerant bacterium LysiniBacillus sphaericus strain OT4b.31. Standards in Genomic Sciences 9, 42-56.
186. Perez, et al., (2005). Bacillus thuringiensis subsp. israelensis Cyt1Aa synergizes Cry11Aa toxin by functioning as a membrane-bound receptor. Proceedings of the National Academy of Sciences of the United States of America 102, 18303-18308.
187. Pognonec, et al., (1991). A quick procedure for purification of functional recombinant proteins over-expressed in E. coli. Nucleic Acids Research 19, 6650.
188. Poncet, S., Delécluse, A., Klier, A., and Rapoport, G. (1995). Evaluation of synergistic interactions among the CryIVA, CryIVB, and CryIVD Toxic Components of B. thuringiensis subsp. israelensis Crystals. Journal of Invertebrate Pathology 66, 131-135.
189. Poopathi, S., and Abidha, S. (2010). Mosquitocidal bacterial toxins (Bacillus sphaericus and B. thuringiensis serovar israelensis): Mode of action, cytopathological effects and mechanism of resistance. Journal of Physiology and Pathophysiology 1, 22-38.
190. Porter, A. G., Davidson, E. W., and Liu, J. W. (1993). Mosquitocidal toxins of bacilli and their genetic manipulation for effective biological control of mosquitoes. Microbiological Reviews 57, 838-861.
191. Promdonkoy, B., and Ellar, D. J. (2000). Membrane pore architecture of a cytolytic toxin from Bacillus thuringiensis. Biochemical Journal 350, 275-282.
192. Promdonkoy, B., Promdonkoy, P., and Panyim, S. (2005). Co-expression of Bacillus thuringiensis Cry4Ba and Cyt2Aa2 in Escherichia coli revealed high synergism against Aedes aegypti and Culex quinquefasciatus larvae. FEMS Microbiology Letters 252, 121-126.
193. Pröschold, T., Harris, E. H., and Coleman, A. W. (2005). Portrait of a Species: Chlamydomonas reinhardtii. Genetics 170, 1601-1610.
194. Puigbò, P., Guzman, E., Romeu, A., and Garcia-Vallvé, S. (2007). OPTIMIZER: a web server for optimizing the codon usage of DNA sequences. Nucleic Acids Research 35, W126-W131.
195. Purton, S. (2007). Tools and techniques for chloroplast Transformation of Chlamydomonas. In: Transgenic microalgae as green cell factories (Leon, R., Galván, A., and Fernandez, E., eds), Advances in Experimental Medicine and Biology 616, 34-45, Springer, New York.
196. Purton, S., Szaub, J. B., Wannathong, T., Young, R., and Economou, C. K. (2013). Genetic engineering of algal chloroplasts: Progress and prospects. Russian Journal of Plant Physiology 60, 491-499.
197. Pusztai, et al., (1991). The mechanism of sunlight-mediated inactivation of Bacillus thuringiensis crystals. Biochemical Journal 273, 43-47.
198. Quinn, J. M., and Merchant, S. (1998). Copper-responsive gene expression during adaptation to copper deficiency. In: Photosynthesis: Molecular Biology of Energy Capture, Methods in Enzymology, 297, 263-279 (Lee, M., ed), Academic Press, New York.
199. Quintana-Castro, et al., (2005). Expression of the cry11A gene of Bacillus thuringiensis ssp. israelensis in Saccharomyces cerevisiae. Canadian Journal of Microbiology 51, 165-170.
200. Qureshi, N., Chawla, S., Likitvivatanavong, S., Lee, H. L., and Gill, S. S. (2014). The cry toxin operon of Clostridium bifermentans subsp. malaysia is highly toxic to Aedes larval mosquitoes. Applied and Environmental Microbiology 80, 5689-5697.
201. Raghavendra, K., Barik, T., Reddy, B., Sharma, P., and Dash, A. (2011). Malaria vector control: from past to future. Parasitology Research 108, 757-779.
202. Ramoska, W. A., Watts, S., and Rodriguez, R. E. (1982). Influence of suspended particulates on the activity of Bacillus thuringiensis serotype h-14 against mosquito larvae. Journal of Economic Entomology 75, 1-4.
203. Ramundo, S., Rahire, M., Schaad, O., and Rochaix, J.-D. (2013). Repression of essential chloroplast genes reveals new signaling pathways and regulatory feedback loops in Chlamydomonas. The Plant Cell 25, 167-186.
204. Rasala, et al., (2010). Production of therapeutic proteins in algae, analysis of expression of seven human proteins in the chloroplast of Chlamydomonas reinhardtii. Plant Biotechnology Journal 8, 719-733.
205. Rasala, B. A., Muto, M., Sullivan, J., and Mayfield, S. P. (2011). Improved heterologous protein expression in the chloroplast of Chlamydomonas reinhardtii through promoter and 5′ untranslated region optimization. Plant Biotechnology Journal 9, 674-683.
206. Rasala, et al., (2013). Expanding the spectral palette of fluorescent proteins for the green microalga Chlamydomonas reinhardtii. The Plant Journal 74, 545-556.
207. Rasala, B. A., Chao, S.-S., Pier, M., Barrera, D. J., and Mayfield, S. P. (2014a). Enhanced genetic tools for engineering multigene traits into green algae. PLoS ONE 9, e94028.
208. Rasala, B. A., and Mayfield, S. (2014b). Photosynthetic biomanufacturing in green algae; production of recombinant proteins for industrial, nutritional, and medical uses. Photosynthesis Research 123, 1-13.
209. Rochaix, J.-D. (1995). Chlamydomonas Reinhardtii as the photosynthetic yeast. Annual Review of Genetics 29, 209-230.
210. Rochaix, J.-D. (1996). Post-transcriptional regulation of chloroplast gene expression in Chlamydomonas reinhardtii. Plant Molecular Biology 32, 327-341.
211. Rosales-Mendoza, S., Paz-Maldonado, L., and Soria-Guerra, R. (2011). Chlamydomonas reinhardtii as a viable platform for the production of recombinant proteins: current status and perspectives. Plant Cell Reports 31, 479-494.
212. Rosso, M. L., and Delécluse, A. (1997). Contribution of the 65-kilodalton protein encoded by the cloned gene cry19A to the mosquitocidal activity of Bacillus thuringiensis subsp. jegathesan. Applied and Environmental Microbiology 63, 4449-4455.
213. Saengwiman, et al., (2011). In vivo identification of Bacillus thuringiensis Cry4Ba toxin receptors by RNA interference knockdown of glycosylphosphatidylinositol-linked aminopeptidase N transcripts in Aedes aegypti larvae. Biochemical and Biophysical Research Communications 407, 708-713.
214. Saiful, A. N., Lau, M. S., Sulaiman, S., and Hidayatulfathi, O. (2012). Residual effects of TMOF-Bti formulations against 1^{st} instar Aedes aegypti Linnaeus larvae outside laboratory. Asian Pacific Journal of Tropical Biomedicine 2, 315-319.
215. Schnepf, et al., (1998). Bacillus thuringiensis and its pesticidal crystal proteins. Microbiology and Molecular Biology Reviews 62, 775-806.
216. Schwartz, J. L., Potvin, L., Coux, F., Charles, J. F., Berry, C., Humphreys, M. J., Jones, A. F., Bernhart, I., Dalla Serra, M., and Menestrina, G. (2001). Permeabilization of model lipid membranes by bacillus sphaericus mosquitocidal binary toxin and its individual components. The Journal of Membrane Biology 184, 171-183.
217. Shadduck, J. A., Singer, S., and Lause, S. (1980). Lack of mammalian pathogenicity of entomocidal isolates of Bacillus sphaericus. Environmental Entomology 9, 403-407
218. Sharp, P. M., and Li, W. H. (1987). The codon adaptation index—a measure of directional synonymous codon usage bias, and its potential applications. Nucleic Acids Research 15, 1281-1295.
219. Siegel, J. P. and Shadduck, J. (1990) Mammalian safety of Bacillus thuringiensis israelensis. In: Bacterial Control of Mosquitoes and black flies: biochemistry, genetics, & applications of Bacillus thuringiensis israelensis and Bacillus sphaericus (De Barjac, H., and Sutherland, D., eds), 202-217, Rutgers University Press, New Brunswick, N.J.
220. Siegel, J. P. (2001). The Mammalian safety of Bacillus thuringiensis-based insecticides. Journal of Invertebrate Pathology 77, 13-21.
221. Silflow, C. D., and Lefebvre, P. A. (2001). Assembly and motility of eukaryotic cilia and flagella. lessons from Chlamydomonas reinhardtii. Plant Physiology 127, 1500-1507.
222. Silva-Filha, M.-H., Regis, L., Nielsen-Leroux, C., and Charles, J.-F. (1995). Low-level resistance to Bacillus sphaericus in a field-treated population of Culex quinquefasciatus (Diptera: Culicidae). Insecticide Resistance and Resistance Management 88, 525-530
223. Silva-Filha, et al., (2004). Two Bacillus sphaericus binary toxins share the midgut receptor binding site: implications for resistance of Culex pipiens complex (Diptera: Culicidae) larvae. FEMS Microbiology Letters 241, 185-191.
224. Soberón, et al., (2010). Pore formation by Cry toxins. In: Proteins: Membrane Binding and Pore Formation, Advances in Experimental Medicine and Biology 677 (Anderluh, G. and Lakey, J., eds), 127-142, Landes Bioscience, Austin, Tex.
225. Soberón, M., López-Diaz, J. A., and Bravo, A. (2013). Cyt toxins produced by Bacillus thuringiensis: A protein fold conserved in several pathogenic microorganisms. Peptides 41, 87-93.
226. Soltes-Rak, E., Kushner, D. J., Williams, D. D., and Coleman, J. R. (1993). Effect of promoter modification on mosquitocidal cryIVB gene expression in Synechococcus sp. strain PCC 7942. Applied and Environmental Microbiology 59, 2404-2410.
227. Soltes-Rak, E., Kushner, D., Williams, D. D., and Coleman, J. (1995). Factors regulating cryIVB expression in the cyanobacterium Synechococcus PCC 7942. Molecular Genetics and Genomics 246, 301-308.
228. Specht, E., Miyake-Stoner, S., and Mayfield, S. (2010). Micro-algae come of age as a platform for recombinant protein production. Biotechnology Letters 32, 1373-1383.
229. Stenico, M., Lloyd, A. T., and Sharp, P. M. (1994). Codon usage in Caenorhabditis elegans: delineation of translational selection and mutational biases. Nucleic Acids Research 22, 2437-2446.
230. Studier, W., F., Rosenberg, A. H., Dunn, J. J., and Dubendorff, J. W. (1990). Use of T7 RNA polymerase to direct expression of cloned genes. In: Gene Expression Technology, Methods in Enzymology 185 (David. V. G., ed), 60-89, Academic Press, San Diego.
231. Sun, et al., (2013). Identification and characterization of three previously undescribed crystal proteins from Bacillus thuringiensis subsp. jegathesan. Applied and Environmental Microbiology 79, 3364-3370.
232. Surzycki, R., Cournac, L., Peltier, G., and Rochaix, J.-D. (2007). Potential for hydrogen production with inducible chloroplast gene expression in Chlamydomonas. Proceedings of the National Academy of Sciences of the United States of America 104, 17548-17553.
233. Surzycki, et al., (2009). Factors effecting expression of vaccines in microalgae. Biologicals 37, 133-138.
234. Takken, W., and Knols, B. G. J. (2009). Malaria vector control: current and future strategies. Trends in Parasitology 25, 101-104.
235. Tetreau, G., Stalinski, R., David, J.-P., and Després, L. (2013). Monitoring resistance to Bacillus thuringiensis subsp. israelensis in the field by performing bioassays with each Cry toxin separately. Memórias do Instituto Oswaldo Cruz 108, 894-900.
236. Thammasittirong, et al., (2011). Aedes aegypti Membrane-bound alkaline phosphatase expressed in Escherichia coli retains high-affinity binding for Bacillus thuringiensis Cry4Ba toxin. Applied and Environmental Microbiology 77, 6836-6840.
237. Tran, et al., (2013). Production of unique immunotoxin cancer therapeutics in algal chloroplasts. Proceedings of the National Academy of Sciences of the United States of America 110, E15-E22.
238. Uniacke, J., and Zerges, W. (2009). Chloroplast protein targeting involves localized translation in Chlamydomonas. Proceedings of the National Academy of Sciences of the United States of America 106, 1439-1444.
239. Vaeck, et al., (1987). Transgenic plants protected from insect attack. Nature 328, 33-37.
240. Vilarinhos, P., and Monnerat, R. (2004). Larvicidal persistence of formulations of Bacillus thuringiensis var. israelensis to control larval Aedes aegypti. Journal of the American Mosquito Control Association 20, 311-314.
241. Visick, J. E., and Whiteley, H. R. (1991). Effect of a 20-kilodalton protein from Bacillus thuringiensis subsp. israelensis on production of the CytA protein by Escherichia coli. Journal of Bacteriology 173, 1748-1756.
242. Ward, E. S., Ridley, A. R., Ellar, D. J., and Todd, J. A. (1986). Bacillus thuringiensis var. israelensis δ-endotoxin: Cloning and expression of the toxin in sporogenic and asporogenic strains of Bacillus subtilis. Journal of Molecular Biology 191, 13-22.
243. Weill, et al., (2003). Comparative genomics: Insecticide resistance in mosquito vetors. Nature 423, 136-137.
244. WHO. (1999). Microbial pest control agent: Bacillus thuringiensis. World Health Organization, Geneva Switzerland, Available from: Available from: http://apps.who.int/iris/handle/10665/42242
245. WHO. (2005). Guidelines for laboratory and field testing of mosquito larvicides. World Health Organization, Geneva, Available from: http://apps.who.int/iris/handle/10665/69101
246. WHO. (2013). World Malaria Report 2013. World Health Organization, Geneva Switzerland, Available from: http://www.who.int/malaria/publications/world_malaria_report_2013/en/
247. Wintermans, J. F. G. M., and De Mots, A. (1965). Spectrophotometric characteristics of chlorophylls a and b and their phenophytins in ethanol. Biochimica et Biophysica Acta (BBA)—Biophysics including Photosynthesis 109, 448-453.
248. Wirth, M. C., Georghiou, G. P., and Federici, B. A. (1997). CytA enables CryIV endotoxins of Bacillus thuringiensis to overcome high levels of CryIV resistance in the mosquito, Culex quinquefasciatus. Proceedings of the National Academy of Sciences of the United States of America 94, 10536-10540.
249. Wirth, M. C., Georghiou, G. P., Malik, J. I., and Hussain, G. (2000). Laboratory selection for resistance to Bacillus sphaericusin Culex quinquefasciatus (Diptera: Culicidae) from California, USA. Journal of Medical Entomology 37, 534-540.
250. Wirth, M. C., Jiannino, J. A., Federici, B. A., and Walton, W. E. (2004). Synergy between Toxins of Bacillus thuringiensis subsp. israelensis and Bacillus sphaericus. Journal of Medical Entomology 41, 935-941.
251. Wirth, M. C., Walton, W. E., and Federici, B. A. (2010). Evolution of resistance to the Bacillus sphaericus Bin toxin is phenotypically masked by combination with the mosquitocidal proteins of Bacillus thuringiensis subspecies israelensis. Environmental Microbiology 12, 1154-1160.
252. Wraight, S. P., Molloy, D. P., and Singer, S. (1987). Studies on the culicine mosquito host range of Bacillus sphaericus and Bacillus thuringiensis var israelensis with notes on the effects of temperature and instar on bacterial efficacy. Journal of Invertebrate Pathology 49, 291-302.
253. Wu, D., Johnson, J. J., and Federici, B. A. (1994). Synergism of mosquitocidal toxicity between CytA and CryIVD proteins using inclusions produced from cloned genes of Bacillus thuringiensis. Molecular Microbiology 13, 965-972.
254. Xiaoqiang, et al., (1997). Mosquito larvicidal activity of transgenic Anabaena strain PCC 7120 expressing combinations of genes from Bacillus thuringiensis subsp. israelensis. Applied and Environmental Microbiology 63, 4971-4974.
255. Xiong, F., Komenda, J., Kopecky, J., and Nedbal, L. (1997). Strategies of ultraviolet-B protection in microscopic algae. Physiologia Plantarum 100, 378-388.
256. Xu, C., Wang, B.-C., Yu, Z., and Sun, M. (2014). Structural insights into Bacillus thuringiensis Cry, Cyt and Parasporin Toxins. Toxins 6, 2732-2770.
257. Xu, Y., Nagai, M., Bagdasarian, M., Smith, T. W., and Walker, E. D. (2001). Expression of the p20 gene from Bacillus thuringiensis H-14 Increases Cry11A toxin production and enhances mosquito-larvicidal activity in recombinant gram-negative bacteria. Applied and Environmental Microbiology 67, 3010-3015.
258. Yamagiwa, et al., (1999). Activation process of dipteran-specific insecticidal protein produced by Bacillus thuringiensis subsp. israelensis. Applied and Environmental Microbiology 65, 3464-3469.
259. Yamagiwa, et al., (2002). Active form of dipteran-Specific insecticidal protein Cry11A produced by Bacillus thuringiensis subsp. israelensis. Bioscience, Biotechnology, and Biochemistry 66, 516-522.
260. Yamagiwa, M., Sakagawa, K., and Sakai, H. (2004). Functional analysis of two processed fragments of Bacillus thuringiensis Cry11A toxin. Bioscience, Biotechnology, and Biochemistry 68, 523-528.
261. Yap, W. H., Thanabalu, T., and Porter, A. G. (1994a). Expression of mosquitocidal toxin genes in a gas-vacuolated strain of Ancylobacter aquaticus. Applied and Environmental Microbiology 60, 4199-4202.
262. Yap, W. H., Thanabalu, T., and Porter, A. G. (1994b). Influence of transcriptional and translational control sequences on the expression of foreign genes in Caulobacter crescentus. Journal of Bacteriology 176, 2603-2610.
263. Yoshida, et al., (1989). Insecticidal activity of a peptide containing the 30^{th} to 695^{th} amino acid residues of the 130-kDa protein of Bacillus thuringiensis var. israelensis. Agricultural and Biological Chemistry 53, 2121-2127
264. Zaritsky, et al., (2010). Transgenic organisms expressing genes from Bacillus thuringiensis to combat insect pests. Bioengineered Bugs 1, 341-344.
265. Zedler, et al., (2014). Stable expression of a bifunctional diterpene synthase in the chloroplast of Chlamydomonas reinhardtii. Journal of Applied Phycology 26, 1-7.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in biological control, biochemistry, molecular biology, entomology, plankton, fishery systems, and fresh water ecology, or related fields are intended to be within the scope of the following claims.

1. A composition comprising a Chlamydomonas chloroplast having a codon-modified cyt1Aa nucleic acid gene sequence in operable combination with a heterologous promoter, wherein said chloroplast expresses a cyt1Aa protoxin.

2. The composition of claim 1, wherein said codon-modified nucleic acid sequence is SEQ ID NO:18.

3. The composition of claim 1, further comprising a codon-modified cry11Aa nucleic acid gene sequence, wherein said chloroplast expresses a Cry11Aa protein.

4. The composition of claim 1, further comprising a codon-modified cry4Aa nucleic acid gene sequence, wherein said chloroplast expresses a Cry4Aa protein.

5. The composition of claim 1, further comprising a codon-modified gene encoding a starch-binding domain.

6. The composition of claim 1, wherein said Chlamydomonas chloroplast is part of a Chlamydomonas reinhardtii cell.

7. The composition of claim 6, wherein said Chlamydomonas reinhardtii is a wild-type organism.

8. The composition of claim 6, wherein said Chlamydomonas reinhardtii is viable.

9. A method comprising introducing a non-native cyt1Aa gene derived from Bacillus thuringiensi sp. israelensis into a Chlamydomonas chloroplast, said cyt1Aa gene comprising a codon-modified nucleic acid sequence, wherein said cyt1Aa gene is in operable combination with a heterologous promoter.

10. A method comprising introducing a non-native cyt1Aa gene derived from Bacillus thuringiensi sp. israelensis into a Chlamydomonas chloroplast, said cyt1Aa gene comprising a codon-modified nucleic acid sequence, wherein said cyt1Aa gene is in operable combination with a heterologous promoter, under conditions such that said expressed cyt1Aa gene product provides synergistic activity against mosquito larvae in the presence of a Cry protein.

11. The method of claim 10, wherein said Cry protein is selected from the group consisting of Cry4Aa, Cry4Ba, Cry4Aa₇₀₀, Cry4Ba₆₇₅ and Cry11Aa.

12. The method of claim 10, wherein said Chlamydomonas chloroplast further expresses a Cry protoxin non-native gene selected from the group consisting of Cry4Aa, Cry4Ba₆₇₅ and Cry11Aa.

13. The method of claim 10, wherein said promoter is a modified psbD promoter comprising psbD 5′-UTR (psbDₘ).

14. A method of treating a body of water comprising mosquito larvae comprising introducing a larvicidal-Chlamydomonas strain, said strain constitutively expressing a cyt1Aa gene product providing synergistic activity against mosquito larvae in the presence of a Cry protein.

15. The method of claim 14, wherein said Cry protein is selected from the group consisting of Cry4Aa₇₀₀, Cry4Ba₆₇₅ and Cry11Aa.

16. The method of claim 14, wherein said Chlamydomonas chloroplast further expresses a Cry protoxin non-native gene selected from the group consisting of Cry4Aa, Cry4Ba, Cry4Aa₇₀₀, Cry4Ba₆₇₅ and Cry11Aa.

17. The method of claim 16, wherein said Chlamydomonas reinhardtii are toxic to mosquito larvae.

18. The method of claim 14, wherein said body of water is treated with a second Chlamydomonas reinhardtii strain, said second strain expressing a Cry protoxin non-native gene selected from the group consisting of Cry4Aa₇₀₀, Cry4Ba₆₇ₛ and Cry11Aa.

19. (canceled)

20. The composition of claim 1, wherein said codon-modified nucleic acid sequence is SEQ ID NO:01.

21. The composition of claim 1, further comprising a codon-modified cyt1A nucleic acid gene sequence, wherein said chloroplast expresses a Cyt1A protein.

22.-30. (canceled)

31. The method of claim 9, wherein said promoter is a modified psbD promoter comprising psbD 5′-UTR (psbDₘ).

32. The method of claim 13, wherein said cry11Aa gene further comprises a downstream region, wherein said downstream region has a 3′ psbA gene untranslated region.

33. The method of claim 9, wherein said cry11Aa gene further comprises in operable combination a codon modified starch binding domain gene, wherein said gene encodes a starch-binding domain.

34.-36. (canceled)

37. The method of claim 9, wherein said codon-modified nucleic acid sequence is SEQ ID NO:01.

38. The method of claim 9, wherein said gene sequence is in a vector.

39-42. (canceled)


SEQUENCE LISTING (30 sequences)

SEQ ID NO: 1 - DNA, 1938 bp, Artificial Sequence
  Synthetic
atgctcgatatggaagactcatcattagacactttatcaattgtaaacgaaactgacttcccattatacaacaactacactgaaccaactattgctccagctttaattgctgtagctccaattgctcaatacttagctactgctattggtaaatgggctgctaaagctgctttctcaaaagtattatcattaattttcccaggttcacaaccagctactatggaaaaagtacgtactgaagtagaaactttaattaaccaaaaattatcacaagaccgtgtaaacattttaaacgctgaataccgtggtattattgaagtatcagacgtattcgacgcttacattaaacaaccaggtttcactccagctactgctaaaggttacttcttaaacttatcaggtgctattattcaacgtttaccacaattcgaagtacaaacttacgaaggtgtatcaattgctttattcactcaaatgtgtactttacacttaactttattaaaagacggtattttagctggttcagcttggggtttcactcaagctgacgtagactcattcattaaattattcaaccaaaaagtattagactaccgtactcgtttaatgcgtatgtacactgaagaattcggtcgtttatgtaaagtatcattaaaagacggtttaactttccgtaacatgtgtaacttatacgtattcccattcgctgaagcttggtcattaatgcgttacgaaggtttaaaattacaatcatcattatcattatgggactacgtaggtgtatcaattccagtaaactacaacgaatggggtggtttagtatacaaattattaatgggtgaagtaaaccaacgtttaactactgtaaaattcaactactcattcactaacgaaccagctgacattccagctcgtgaaaacattcgtggtgtacacccaatttacgacccatcatcaggtttaactggttggattggtaacggtcgtactaacaacttcaacttcgctgacaacaacggtaacgaaattatggaagtacgtactcaaactttctaccaaaacccaaacaacgaaccaattgctccacgtgacattattaaccaaattttaactgctccagctccagctgacttattcttcaaaaacgctgacattaacgtaaaattcactcaatggttccaatcaactttatacggttggaacattaaattaggtactcaaactgtattatcatcacgtactggtactattccaccaaactacttagcttacgacggttactacattcgtgctatttcagcttgtccacgtggtgtatcattagcttacaaccacgacttaactactttaacttacaaccgtattgaatacgactcaccaactactgaaaacattattgtaggtttcgctccagacaacactaaagacttctactcaaaaaaatcacactacttatcagaaactaacgactcatacgtaattccagctttacaattcgctgaagtatcagaccgttcattcttagaagacactccagaccaagctactgacggttcaattaaattcgctcgtactttcatttcaaacgaagctaaatactcaattcgtttaaacactggtttcaacactgctactcgttacaaattaattattcgtgtacgtgtaccataccgtttaccagctggtattcgtgtacaatcacaaaactcaggtaacaaccgtatgttaggttcattcactgctaacgctaacccagaatgggtagacttcgtaactgacgctttcactttcaacgacttaggtattactacttcatcaactaacgctttattctcaatttcatcagactcattaaactcaggtgaagaatggtacttatcacaattattcttagtaaaagaatcagctttcactactcaaattaacccattattaaaa

SEQ ID NO: 2 - DNA, 2100 bp, Artificial Sequence
  Synthetic
atgaacccataccaaaacaaaaacgaatacgaaactttaaacgcttcacaaaaaaaattaaacatttcaaacaactacactcgttacccaattgaaaactcaccaaaacaattattacaatcaactaactacaaagactggttaaacatgtgtcaacaaaaccaacaatacggtggtgacttcgaaactttcattgactcaggtgaattatcagcttacactattgtagtaggtactgtattaactggtttcggtttcactactccattaggtttagctttaattggtttcggtactttaattccagtattattcccagctcaagaccaatcaaacacttggtcagacttcattactcaaactaaaaacattattaaaaaagaaattgcttcaacttacatttcaaacgctaacaaaattttaaaccgttcattcaacgtaatttcaacttaccacaaccacttaaaaacttgggaaaacaacccaaacccacaaaacactcaagacgtacgtactcaaattcaattagtacactaccacttccaaaacgtaattccagaattagtaaactcatgtccaccaaacccatcagactgtgactactacaacattttagtattatcatcatacgctcaagctgctaacttacacttaactgtattaaaccaagctgtaaaattcgaagcttacttaaaaaacaaccgtcaattcgactacttagaaccattaccaactgctattgactactacccagtattaactaaagctattgaagactacactaactactgtgtaactacttacaaaaaaggtttaaacttaattaaaactactccagactcaaacttagacggtaacattaactggaacacttacaacacttaccgtactaaaatgactactgctgtattagacttagtagctttattcccaaactacgacgtaggtaaatacccaattggtgtacaatcagaattaactcgtgaaatttaccaagtattaaacttcgaagaatcaccatacaaatactacgacttccaataccaagaagactcattaactcgtcgtccacacttattcacttggttagactcattaaacttctacgaaaaagctcaaactactccaaacaacttcttcacttcacactacaacatgttccactacactttagacaacatttcacaaaaatcatcagtattcggtaaccacaacgtaactgacaaattaaaatcattaggtttagctactaacatttacattttcttattaaacgtaatttcattagacaacaaatacttaaacgactacaacaacatttcaaaaatggacttcttcattactaacggtactcgtttattagaaaaagaattaactgctggttcaggtcaaattacttacgacgtaaacaaaaacattttcggtttaccaattttaaaacgtcgtgaaaaccaaggtaacccaactttattcccaacttacgacaactactcacacattttatcattcattaaatcattatcaattccagctacttacaaaactcaagtatacactttcgcttggactcactcatcagtagacccaaaaaacactatttacactcacttaactactcaaattccagctgtaaaagctaactcattaggtactgcttcaaaagtagtacaaggtccaggtcacactggtggtgacttaattgacttcaaagaccacttcaaaattacttgtcaacactcaaacttccaacaatcatacttcattcgtattcgttacgcttcaaacggttcagctaacactcgtgctgtaattaacttatcaattccaggtgtagctgaattaggtatggctttaaacccaactttctcaggtactgactacactaacttaaaatacaaagacttccaatacttagaattctcaaacgaagtaaaattcgctccaaaccaaaacatttcattagtattcaaccgttcagacgtatacactaacactactgtattaattgacaaaattgaattcttaccaattactcgttcaattcgtgaagaccgtgaaaaacaaaaattagaaactgtacaacaaattattaacactttc

SEQ ID NO: 3 - DNA, 3408 bp, Artificial Sequence
  Synthetic
atgcaatcaggttacccattagctaacgacttacaaggttcaatgaaaaacactaactacaaagactggttagctatgtgtgaaaacaaccaacaatacggtgtaaacccagctgctattaactcatcatcagtatcaactgctttaaaagtagctggtgctattttaaaattcgtaaacccaccagctggtactgtattaactgtattatcagctgtattaccaattttatggccaactaacactccaactccagaacgtgtatggaacgacttcatgactaacactggtaacttaattgaccaaactgtaactgcttacgtacgtactgacgctaacgctaaaatgactgtagtaaaagactacttagaccaatacactactaaattcaacacttggaaacgtgaaccaaacaaccaatcataccgtactgctgtaattactcaattcaacttaacttcagctaaattacgtgaaactgctgtatacttctcaaacttagtaggttacgaattattattattaccaatttacgctcaagtagctaacttcaacttattattaattcgtgacggtttaattaacgctcaagaatggtcattagctcgttcagctggtgaccaattatacaacactatggtacaatacactaaagaatacattgctcactcaattacttggtacaacaaaggtttagacgtattacgtaacaaatcaaacggtcaatggattactttcaacgactacaaacgtgaaatgactattcaagtattagacattttagctttattcgcttcatacgacccacgtcgttacccagctgacaaaattgacaacactaaattatcaaaaactgaattcactcgtgaaatttacactgctttagtagaatcaccatcatcaaaatcaattgctgctttagaagctgctttaactcgtgacgtacacttattcacttggttaaaacgtgtagacttctggactaacactatttaccaagacttacgtttcttatcagctaacaaaattggtttctcatacactaactcatcagctatgcaagaatcaggtatttacggttcatcaggtttcggttcaaacttaactcaccaaattcaattaaactcaaacgtatacaaaacttcaattactgacacttcatcaccatcaaaccgtgtaactaaaatggacttctacaaaattgacggtactttagcttcatacaactcaaacattactccaactccagaaggtttacgtactactttcttcggtttctcaactaacgaaaacactccaaaccaaccaactgtaaacgactacactcacattttatcatacattaaaactgacgtaattgactacaactcaaaccgtgtatcattcgcttggactcacaaaattgtagacccaaacaaccaaatttacactgacgctattactcaagtaccagctgtaaaatcaaacttcttaaacgctactgctaaagtaattaaaggtccaggtcacactggtggtgacttagtagctttaacttcaaacggtactttatcaggtcgtatggaaattcaatgtaaaacttcaattttcaacgacccaactcgttcatacggtttacgtattcgttacgctgctaactcaccaattgtattaaacgtatcatacgtattacaaggtgtatcacgtggtactactatttcaactgaatcaactttctcacgtccaaacaacattattccaactgacttaaaatacgaagaattccgttacaaagacccattcgacgctattgtaccaatgcgtttatcatcaaaccaattaattactattgctattcaaccattaaacatgacttcaaacaaccaagtaattattgaccgtattgaaattattccaattactcaatcagtattagacgaaactgaaaaccaaaacttagaatcagaacgtgaagtagtaaacgctttattcactaacgacgctaaagacgctttaaacattggtactactgactacgacattgaccaagctgctaacttagtagaatgtatttcagaagaattatacccaaaagaaaaaatgttattattagacgaagtaaaaaacgctaaacaattatcacaatcacgtaacgtattacaaaacggtgacttcgaatcagctactttaggttggactacttcagacaacattactattcaagaagacgacccaattttcaaaggtcactacttacacatgtcaggtgctcgtgacattgacggtactattttcccaacttacattttccaaaaaattgacgaatcaaaattaaaaccatacactcgttacttagtacgtggtttcgtaggttcatcaaaagacgtagaattagtagtatcacgttacggtgaagaaattgacgctattatgaacgtaccagctgacttaaactacttatacccatcaactttcgactgtgaaggttcaaaccgttgtgaaacttcagctgtaccagctaacattggtaacacttcagacatgttatactcatgtcaatacgacactggtaaaaaacacgtagtatgtcaagactcacaccaattctcattcactattgacactggtgctttagacactaacgaaaacattggtgtatgggtaatgttcaaaatttcatcaccagacggttacgcttcattagacaacttagaagtaattgaagaaggtccaattgacggtgaagctttatcacgtgtaaaacacatggaaaaaaaatggaacgaccaaatggaagctaaacgttcagaaactcaacaagcttacgacgtagctaaacaagctattgacgctttattcactaacgtacaagacgaagctttacaattcgacactactttagctcaaattcaatacgctgaatacttagtacaatcaattccatacgtatacaacgactggttatcagacgtaccaggtatgaactacgacatttacgtagaattagacgctcgtgtagctcaagctcgttacttatacgacactcgtaacattattaaaaacggtgacttcactcaaggtgtaatgggttggcacgtaactggtaacgctgacgtacaacaaattgacggtgtatcagtattagtattatcaaactggtcagctggtgtatcacaaaacgtacacttacaacacaaccacggttacgtattacgtgtaattgctaaaaaagaaggtccaggtaacggttacgtaactttaatggactgtgaagaaaaccaagaaaaattaactttcacttcatgtgaagaaggttacattactaaaactgtagacgtattcccagacactgaccgtgtacgtattgaaattggtgaaactgaaggttcattctacattgaatcaattgaattaatttgtatgaacgaa

SEQ ID NO: 4 - DNA, 21 bp, Artificial Sequence
  Synthetic
gtcaacaaaaccaacaatacg

SEQ ID NO: 5 - DNA, 21 bp, Artificial Sequence
  Synthetic
ttagtgtagtcagtacctgag

SEQ ID NO: 6 - DNA, 21 bp, Artificial Sequence
  Synthetic
aacgacttacaaggttcaatg

SEQ ID NO: 7 - DNA, 21 bp, Artificial Sequence
  Synthetic
tgtctgggaatacgtctacag

SEQ ID NO: 8 - DNA, 21 bp, Artificial Sequence
  Synthetic
ggaagactcatcattagacac

SEQ ID NO: 9 - DNA, 21 bp, Artificial Sequence
  Synthetic
agtagcagtgttgaaaccagt

SEQ ID NO: 10 - DNA, 24 bp, Artificial Sequence
  Synthetic
gaaattaatacgactcactatagg

SEQ ID NO: 11 - DNA, 19 bp, Artificial Sequence
  Synthetic
gctagttattgctcagcgg

SEQ ID NO: 12 - DNA, 39 bp, Artificial Sequence
  Synthetic
gctcccatggatcctcataataataaaacctttattcat

SEQ ID NO: 13 - DNA, 47 bp, Artificial Sequence
  Synthetic
ccggcatatggtgtatctttaaaataaaaaaacaactcatcgttacg

SEQ ID NO: 14 - DNA, 36 bp, Artificial Sequence
  Synthetic
cggggctgagctcaaacaactaatttttttttaaac

SEQ ID NO: 15 - DNA, 37 bp, Artificial Sequence
  Synthetic
cagtgctcagcggatcctgccaactgcctatggtagc

SEQ ID NO: 16 - DNA, 20 bp, Artificial Sequence
  Synthetic
tggaattggatatggactag

SEQ ID NO: 17 - DNA, 20 bp, Artificial Sequence
  Synthetic
ggtacttgcatttcataagt

SEQ ID NO: 18 - DNA, 774 bp, Artificial Sequence
  Synthetic
atggaaaatttaaatcattgtccattagaagatattaaagttaatccatggaaaacaccacaatcaacagctcgtgttattacattacgtgttgaagatccaaatgaaattaataatttattatcaattaatgaaattgataatccaaattatattttacaagctattatgttagctaatgcttttcaaaatgctttagttccaacatcaacagattttggtgatgctttacgtttttcaatgccaaaaggtttagaaattgctaatacaattacaccaatgggtgctgttgtttcttatgttgatcaaaatgttacacaaacaaataatcaagtttcagttatgattaataaagttttagaagttttaaaaacagttttaggtgttgctttatcaggttcagttattgatcaattaacagctgctgttacaaatacatttacaaatttaaatacacaaaaaaatgaagcttggattttttggggtaaagaaacagctaatcaaacaaattatacatataatgttttatttgctattcaaaatgctcaaacaggtggtgttatgtattgtgttccagttggttttgaaattaaagtttcagctgttaaagaacaagttttattttttacaattcaagattcagcttcatataatgttaatattcaatcattaaaatttgctcaaccattagtttcatcatcacaatatccaattgctgatttaacatcagctattaatggtacattagactacaaagacgacgacgacaaataa

SEQ ID NO: 19 - DNA, 2512 bp, Artificial Sequence
  Synthetic
agaaaacattgctgtgagttgccaagatactgtctgcgtagatcaagttttgtattgcagtgtagattgtttgccagattgtgatattaattgtgataatgtaaaaatttgcgatgtgagcattgaaccaattggagattgtgattgtcacgcggtgaaaattaaagggaaattttcacttcactataaataaaaaatccctaattattaaatgaataataaggtcataatttatgaataaaaatatgacctttaaaataaaaaaattcaataaaaggtggaatgaattatatggaagatagttctttagatactttaagtatagttaatgaaacagactttccattatataataattataccgaacctactattgcgccagcattaatagcagtagctcccatcgcacaatatcttgcaacagctatagggaaatgggcggcaaaggcagcattttcaaaagtactatcacttatattcccaggttctcaacctgctactatggaaaaagttcgtacagaagtggaaacacttataaatcaaaaattaagccaagatcgagtcaatatattaaacgcagaatatagggggattattgaggttagtgatgtatttgatgcgtatattaaacaaccaggttttacccctgcaacagccaagggttattttctaaatctaagtggtgctataatacaacgattacctcaatttgaggttcaaacatatgaaggagtatctatagcactttttactcaaatgtgtacacttcatttaactttattaaaagacggaatcctagcagggagtgcatggggatttactcaagctgatgtagattcatttataaaattatttaatcaaaaagtattagattacaggaccagattaatgagaatgtacacagaagagttcggaagattgtgtaaagtcagtcttaaagatggattgacgttccggaatatgtgtaatttatatgtgtttccatttgctgaagcctggtctttaatgagatatgaaggattaaaattacaaagctctctatcattatgggattatgttggtgtctcaattcctgtaaattataatgaatggggaggactagtttataagttattaatgggggaagttaatcaaagattaacaactgttaaatttaattattctttcactaatgaaccagctgatataccagcaagagaaaatattcgtggcgtccatcctatatacgatcctagttctgggcttacaggatggataggaaacggaagaacaaacaattttaattttgctgataacaatggcaatgaaattatggaagttagaacacaaactttttatcaaaatccaaataatgagcctatagcgcctagagatattataaatcaaattttaactgcgccagcaccagcagacctattttttaaaaatgcagatataaatgtaaagttcacacagtggtttcagtctactctatatgggtggaacattaaactcggtacacaaacggttttaagtagtagaaccggaacaataccaccaaattatttagcatatgatggatattatattcgtgctatttcagcttgcccaagaggagtctcacttgcatataatcacgatcttacaacactaacatataatagaatagagtatgattcacctactacagaaaatattattgtagggtttgcaccagataatactaaggacttttattctaaaaaatctcactatttaagtgaaacgaatgatagttatgtaattcctgctctgcaatttgctgaagtttcagatagatcatttttagaagatacgccagatcaagcaacagacggcagtattaaatttgcacgtactttcattagtaatgaagctaagtactctattagactaaacaccgggtttaatacggcaactagatataaattaattatcagggtaagagtaccttatcgcttacctgctggaatacgggtacaatctcagaattcgggaaataatagaatgctaggcagttttactgcaaatgctaatccagaatgggtggattttgtcacagatgcatttacatttaacgatttagggattacaacttcaagtacaaatgctttatttagtatttcttcagatagtttaaattctggagaagagtggtatttatcgcagttgtttttagtaaaagaatcggcctttacgacgcaaattaatccgttactaaagtagaagtcatgttagcacaagaggagtgagtattgtggctcctcttgtaattttaatcgctaatatttctaatagatataaattatatataatatttaaaaagttataattatgtaattgtagaaaatcatgaatttttcaattttattgacgaggaaacagagtatacgagtttataatttctaataattgtttaaaacatatgcttagaagtcaatttatattagctttacttttagtagaatttataattaatatttaggataaaattggaggataattgatgacagaa

SEQ ID NO: 20 - DNA, 1976 bp, Artificial Sequence
  Synthetic
tatttttttattatgtacgaaaaaaagcattcatctttcaagtagatgaatgcaaaaattaatttgaaatttaatgtatttttataagtggccccaaaaagaaggaatcgttgccgtgccccctgtacaggcagaaccacaatctgataaaggactccatggaaattgaggatcggaggtaatcgcaaaagctcgaagtattaagatttgaaatcgattgttgatttccctgcatattctttccctcattttgtttgatgaaaatctattttcaaatcctaaatcagttcatctattaatcatcataacttggatcacaattgtagtttggatagtttaaatggtgataattattattggataaacgttctatactaatgaaattgatatttgtataaattttatgtcctctagatatctattttttatgttttctatatattttttgtaccagaattaataaatgcagaaattaaaaaccatggagaaaactttctccatggtttttaaagctttagttattttttattaatcactcgttcatgcaaattaattcaatgctttcgatataaaacgaaccttcggtttcgcctatctcaattcgtacacgatctgtatctgggaatacatctactgtcttcgtaatatatccttcttcacaagacgtaaacgtcaatttttcttgattctcctcacaatccataagcgtgacatacccatttccaggtccttcttttttggcaataacacgtaagacatacccatgattatgttggagatggacattttgagatacgccagcactccaattagatagaaccaatacagaaacaccatctatttgttgtacgtctgcatttccagttacatgccaccccattaccccttgtgtaaaatcaccatttttaataatatttcttgtatcatacaaataacgcgcttgtgccactcgtgcatccaactctacatagatatcataattcatacctggaacatctgacaaccaatcattgtacacatatggaatcgattgtaccaaatactcagcgtactgaatttgagcgagtgtcgtatcaaactgtaaagcctcatcttgtacatttgtgaataaagcatcaatggcttgtttcgctacatcatatgcttgttgtgtttccgaacgttttgcttccatttgatcgttccatttcttctccatgtgtttcacgcgtgacagtgcttccccatctattggcccttcttcaattacttctaaattatctaatgatgcgtatccatctggagaagatattttaaacatgacccaaacccctatattttcatttgtatctaatgcccctgtatcaatagtgaaactaaattgatgggaatcctgacatacgacatgctttttccctgtatcatattggcatgaatacaacatatcagaagtgttcccaatgttagccggcacagcggacgtctcacaacgattagacccttcacaatcaaaggtagaaggatacagatagtttaaatcagctggaacattcatgatggcatcaatttcttccccatagcgtgaaaccactagttctacatctttactacttcctacaaatccccttactaggtaacgtgtatacggttttaattttgattcatcaattttttggaatatataggtcggaaatatcgtaccatcaatgtctctcgccccagacatatgaaggtaatgccctttaaaaataggatcatcttcttgaattgtgatattatcacttgttgtccaaccaagcgtagccgattcaaaatccccgttttgaagtacatttcgagattgactaagttgtttcgcatttttaacttcatctaataacagcattttttcttttggatataattcttcagaaatacattccacaagatttgcggcttgatctatgtcataatctgtaagttctgattgtaaagtgttttttataggatttgcataa

SEQ ID NO: 21 - DNA, 2629 bp, Artificial Sequence
  Synthetic
aatgtattaattatttgttgtactgtttctaatttttgtttctctctatcctctcttatagaacgagtaattggcagaaattcaattttatcaataagtactgttgtgtttgtatatacatccgaacgattaaacacaagagatatgttttgatttggagcaaatttcacctcgttagaaaattctaagtactgaaaatctttatattttaaattcgtataatctgtaccagaaaaagtggggttgagtgccatacccagttctgctacccctgggatactaagatttataacagctcgagtatttgcgcttccatttgaagcataacgaattcttataaaatacgattgttgaaaatttgagtgttgacatgtaattttgaaatgatctttgaaatcaattaaatcccctcctgtatgaccaggtccttgaacaaccttagaagcagtcccaagtgaattcgcttttacagctggaatttgggtagttaaatgtgtataaattgtatttttaggatcaacactagagtgtgtccaagcaaacgtatacacttgagttttatatgttgcagggatactaagacttttaataaatgataaaatatgactatagttatcatatgttggaaaaagggtagggtttccttgattctctcttcgtttaagaattggtaacccgaaaatatttttatttacatcataagttatttgcccagatcctgctgtaagttctttctccaaaagtctagtaccattagttataaaaaaatccattttactaatattattataatcatttagatatttattatctaagcttatgacatttaataaaaaaatataaatatttgttgccaaaccaagagattttaatttatcagttacattgtgatttccaaaaacactagatttttgggatatattatcaagtgtgtaatgaaacatattataatggctggtgaaaaaattattaggagtagtttgcgctttttcataaaaattcaaagaatcaagccaagtaaataaatgcggtctacgtgtaagtgaatcctcttgatattgaaagtcataatatttataggggctttcttcgaagttaagtacctgataaatttctcgagtaagttcagattggacacctattggatatttacctacatcataattaggaaagagtgcaacaagatctaatacagcagtagtcatttttgttcgatacgtattgtatgtgttccagtttatatttccatcaagattactatcaggcgtcgttttaattaaatttaatccttttttataagttgttacacaataattagtgtaatcttctatagctttagtcaatactggataataatcaattgctgttggcaaaggctctaaataatcgaattgtcgattgttttttaaatacgcttcaaatttgacggcttgatttaatacagtcagatgtaagtttgctgcttgtgcataactagataatactagtatgttatagtaatcgcaatcactaggattaggaggacaagagtttacaagctctggaatgacattttgaaaatggtaatgaactagctggatttgtgtccttacatcctgagtattttgtgggtttggattattctcccatgttttaaggtgattatgataagtgctgataacattaaacgacctgtttaaaattttattagcattacttatatatgttgatgctatttctttttttataatatttttagtttgtgttataaagtcactccatgtgttagattggtcttgggctggaaaaagaactggtattaatgtaccaaaacctattaaagcaagtcctaagggtgttgtgaacccgaaaccagtcagtacggtcccaactacaatagtataggcactgagttcaccactatcaataaaagtttcaaaatctccaccatactgctgattctgttgacacatattgagccaatctttataatttgtactttgtaataattgttttggactattttctattggatatcttgtataattattagatatatttaattttttttgtgaagcatttaatgtttcatattcatttttattttgataaggattcatatttgttcctcccatactcaatttagatacactctttttctgtagcaacaaagattattttaaatcatttttaaattgatatggtttaaaaagtacaaaattgaaaattattgattacttttacaaatcctatatacatattaatgtaccaatataattattcgtaatttatacattttaaaaatttttgcgttaaatttttaaaactttgtatttcatatggtttgttacaaaacctcacacaaaaataagaaaaccttctttacaagaattcttggtatctttgacccttatgcatttatcctctcctatgtagtaatctctctttcttttacactccaagctatcaaaatttcccttatgcattttaaagtattcgtaatttaaataatctattcctgttacattctttcaacaaataaccgcgtcattttttgacaatcaaccagcctgttaatttttaaaaaagctatctaatccccttcaatatccctttatatgccttttacatcaaatagtataggaactga

SEQ ID NO: 22 - DNA, 513 bp, Artificial Sequence
  Synthetic
ttaatctttggattgttattatagctgtttttttgttgtatactcccgaaaatcgatttgaattttctgaatatcgaacaatatattattttggatgcttgataaccactaacgatatgtatggaaaattattttgaagtgaaaaaatatggtcaaataaaaatggaataattatattggtacagaaatatgattgggattagtgagtctataatatagaaaggaatgttttgttttttgtatataagttgaaaaagatttctgtaaattgtccagagactgtatgtgtagattgagtattggaacatatcgttaattttatattttaatataatgatatgaattatacaaggtctagataagaattgttcataggaatccgtatcaattttttcaaggaatatgtatttgcacttttggtctttttaaatcgtatgaattcaaaatagtttatatcaatctttgttacaccagaaaaagattgtatccaatgtgaatatgggaggaataaat

SEQ ID NO: 23 - DNA, 3922 bp, Artificial Sequence
  Synthetic
atgaattcaggctatccgttagcgaatgacttacaagggtcaatgaaaaacacgaactataaagattggctagccatgtgtgaaaataaccaacagtatggcgttaatccagctgcgattaattcttcttcagttagtaccgctttaaaagtagctggagctatccttaaatttgtaaacccacctgcaggtactgtcttaaccgtacttagcgcggtgcttcctattctttggccgactaatactccaacgcctgaaagagtttggaatgatttcatgaccaatacagggaatcttattgatcaaactgtaacagcttatgtacgaacagatgcaaatgcaaaaatgacggttgtgaaagattatttagatcaatatacaactaaatttaacacttggaaaagagagcctaataaccagtcctatagaacagcagtaataactcaatttaacttaaccagtgccaaacttcgagagaccgcagtttattttagcaacttagtaggttatgaattattgttattaccaatatacgcacaagtagcaaatttcaatttacttttaataagagatggcctcataaatgcacaagaatggtctttagcacgtagtgctggtgaccaactatataacactatggtgcagtacactaaagaatatattgcacatagcattacatggtataataaaggtttagatgtacttagaaataaatctaatggacaatggattacgtttaatgattataaaagagagatgactattcaagtattagatatactcgctctttttgccagttatgatccacgtcgataccctgcggacaaaatagataatacgaaactatcaaaaacagaatttacaagagagatttatacagctttagtagaatctccttctagtaaatctatagcagcactggaggcagcacttacacgagatgttcatttattcacttggctaaagagagtagatttctggaccaatactatatatcaagatttaagatttttatctgccaataaaattgggttttcatatacaaattcttctgcaatgcaagaaagtggaatttatggaagttctggttttggttcaaatcttactcatcaaattcaacttaattctaatgtttataaaacttctatcacagatactagctccccctctaatcgagttacaaaaatggatttctacaaaattgatggtactcttgcctcttataattcaaatataacaccaactcctgaaggtttaaggaccacattttttggattttcaacaaatgagaacacacctaatcaaccaactgtaaatgattatacgcatattttaagctatataaaaactgatgttatagattataacagtaacagggtttcatttgcttggacacataagattgttgaccctaataatcaaatatacacagatgctatcacacaagttccggccgtaaaatctaacttcttgaatgcaacagctaaagtaatcaagggacctggtcatacagggggggatctagttgctcttacaagcaatggtactctatcaggcagaatggagattcaatgtaaaacaagtatttttaatgatcctacaagaagttacggattacgcatacgttatgctgcaaatagtccaattgtattgaatgtatcatatgtattacaaggagtttctagaggaacaacgattagtacagaatctacgttttcaagacctaataatataatacctacagatttaaaatatgaagagtttagatacaaagatccttttgatgcaattgtaccgatgagattatcttctaatcaactgataactatagctattcaaccattaaacatgacttcaaataatcaagtgattattgacagaatcgaaattattccaatcactcaatctgtattagatgagacagagaaccaaaatttagaatcagaacgagaagttgtgaatgcactgtttacaaatgacgcgaaagatgcattaaacattggaacgacagattatgacatagatcaagccgcaaatcttgtggaatgtatttctgaagaattatatccaaaagaaaaaatgctgttattagatgaagttaaaaatgcgaaacaacttagtcaatctcgaaatgtacttcaaaacggggattttgaatcggctacgcttggttggacaacaagtgataatatcacaattcaagaagatgatcctatttttaaagggcattaccttcatatgtctggggcgagagacattgatggtacgatatttccgacctatatattccaaaaaattgatgaatcaaaattaaaaccgtatacacgttacctagtaaggggatttgtaggaagtagtaaagatgtagaactagtggtttcacgctatggggaagaaattgatgccatcatgaatgttccagctgatttaaactatctgtatccttctacctttgattgtgaagggtctaatcgttgtgagacgtccgctgtgccggctaacattgggaacacttctgatatgttgtattcatgccaatatgatacagggaaaaagcatgtcgtatgtcaggattcccatcaatttagtttcactattgatacaggggcattagatacaaatgaaaatataggggtttgggtcatgtttaaaatatcttctccagatggatacgcatcattagataatttagaagtaattgaagaagggccaatagatggggaagcactgtcacgcgtgaaacacatggagaagaaatggaacgatcaaatggaagcaaaacgttcggaaacacaacaagcatatgatgtagcgaaacaagccattgatgctttattcacaaatgtacaagatgaggctttacagtttgatacgacactcgctcaaattcagtacgctgagtatttggtacaatcgattccatatgtgtacaatgattggttgtcagatgttccaggtatgaattatgatatctatgtagagttggatgcacgagtggcacaagcgcgttatttgtatgatacaagaaatattattaaaaatggtgattttacacaaggggtaatggggtggcatgtaactggaaatgcagacgtacaacaaatagatggtgtttctgtattggttctatctaattggagtgctggcgtatctcaaaatgtccatctccaacataatcatgggtatgtcttacgtgttattgccaaaaaagaaggacctggaaatgggtatgtcacgcttatggattgtgaggagaatcaagaaaaattgacgtttacgtcttgtgaagaaggatatattacgaagacagtagatgtattcccagatacagatcgtgtacgaattgagataggcgaaaccgaaggttcgttttatatcgaaagcattgaattaatttgcatgaacgagtgattaataaaaaataactaaagctttaaaaaccatggagaaagttttctccatggtttttaatttctgcatttattaattctggtacaaaaaatatatagaaaacataaaaaatagatatctagaggacataaaatttatacaaatatcaatttcattagtatagaacgtttatccaataataattatcaccatttaaactatccaaactacaattgtgatccaagttatgatgattaatagatgaactgatttaggatttgaaaatagattttcatcaaacaaaatgagggaaagaatatgcagggaaatcaacaatcgatttcaaatcttaatacttcgggattatttcggggtacagccgagatacgtgtaagaatagaccgtatccttacctttatttctatattggatttattgagcttataaatattgtttctgtggtttcgtctatttttattaagtaaattgtctattatgggttaaccctaatcattcattagttactgaaaac

SEQ ID NO: 24 - protein, 8 aa, Artificial Sequence
  Synthetic
DYKDDDDK

SEQ ID NO: 25 - DNA, 108 bp, Bacillus thuringiensis serovar israelensis
atggaagatagttctttagatactttaagtatagttaatgaaacagactttccattatataataattataccgaacctactattgcgccagcattaatagcagtagct

SEQ ID NO: 26 - DNA, 108 bp, Artificial Sequence
  Synthetic
atggaagactcatcattagacactttatcaattgtaaacaggactgacttcccattatacaacaactacactgaaccaactattgctccagctttaattgctgtagct

SEQ ID NO: 27 - protein, 36 aa, Bacillus thuringiensis serovar israelensis
MEDSSLDTLSIVNETDFPLYNNYTEPTIAPALIAVA

SEQ ID NO: 28 - DNA, 2424 bp, Artificial Sequence
  Synthetic
ggatcctcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgatgtgtttttttattttaaagatacaccatatgctcgatatggaagactcatcattagacactttatcaattgtaaacgaaactgacttcccattatacaacaactacactgaaccaactattgctccagctttaattgctgtagctccaattgctcaatacttagctactgctattggtaaatgggctgctaaagctgctttctcaaaagtattatcattaattttcccaggttcacaaccagctactatggaaaaagtacgtactgaagtagaaactttaattaaccaaaaattatcacaagaccgtgtaaacattttaaacgctgaataccgtggtattattgaagtatcagacgtattcgacgcttacattaaacaaccaggtttcactccagctactgctaaaggttacttcttaaacttatcaggtgctattattcaacgtttaccacaattcgaagtacaaacttacgaaggtgtatcaattgctttattcactcaaatgtgtactttacacttaactttattaaaagacggtattttagctggttcagcttggggtttcactcaagctgacgtagactcattcattaaattattcaaccaaaaagtattagactaccgtactcgtttaatgcgtatgtacactgaagaattcggtcgtttatgtaaagtatcattaaaagacggtttaactttccgtaacatgtgtaacttatacgtattcccattcgctgaagcttggtcattaatgcgttacgaaggtttaaaattacaatcatcattatcattatgggactacgtaggtgtatcaattccagtaaactacaacgaatggggtggtttagtatacaaattattaatgggtgaagtaaaccaacgtttaactactgtaaaattcaactactcattcactaacgaaccagctgacattccagctcgtgaaaacattcgtggtgtacacccaatttacgacccatcatcaggtttaactggttggattggtaacggtcgtactaacaacttcaacttcgctgacaacaacggtaacgaaattatggaagtacgtactcaaactttctaccaaaacccaaacaacgaaccaattgctccacgtgacattattaaccaaattttaactgctccagctccagctgacttattcttcaaaaacgctgacattaacgtaaaattcactcaatggttccaatcaactttatacggttggaacattaaattaggtactcaaactgtattatcatcacgtactggtactattccaccaaactacttagcttacgacggttactacattcgtgctatttcagcttgtccacgtggtgtatcattagcttacaaccacgacttaactactttaacttacaaccgtattgaatacgactcaccaactactgaaaacattattgtaggtttcgctccagacaacactaaagacttctactcaaaaaaatcacactacttatcagaaactaacgactcatacgtaattccagctttacaattcgctgaagtatcagaccgttcattcttagaagacactccagaccaagctactgacggttcaattaaattcgctcgtactttcatttcaaacgaagctaaatactcaattcgtttaaacactggtttcaacactgctactcgttacaaattaattattcgtgtacgtgtaccataccgtttaccagctggtattcgtgtacaatcacaaaactcaggtaacaaccgtatgttaggttcattcactgctaacgctaacccagaatgggtagacttcgtaactgacgctttcactttcaacgacttaggtattactacttcatcaactaacgctttattctcaatttcatcagactcattaaactcaggtgaagaatggtacttatcacaattattcttagtaaaagaatcagctttcactactcaaattaacccattattaaaagactacaaagacgacgacgacaaataatctagtcgaggatcgatccggctgctaacaaagcccgaaaggaagctgagttggctgctgccaccgctgagctcaaacaactaatttttttttaaactaaaataaatctggttaaccatacctggtttattttagtttagtttatacacacttttcatatatatatacttaatagctaccataggcagttggcaggatcc

SEQ ID NO: 29 - DNA, 2595 bp, Artificial Sequence
  Synthetic
ggatcctcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttaaagatacaccatatgctcgatatgaacccataccaaaacaaaaacgaatacgaaactttaaacgcttcacaaaaaaaattaaacatttcaaacaactacactcgttacccaattgaaaactcaccaaaacaattattacaatcaactaactacaaagactggttaaacatgtgtcaacaaaaccaacaatacggtggtgacttcgaaactttcattgactcaggtgaattatcagcttacactattgtagtaggtactgtattaactggtttcggtttcactactccattaggtttagctttaattggtttcggtactttaattccagtattattcccagctcaagaccaatcaaacacttggtcagacttcattactcaaactaaaaacattattaaaaaagaaattgcttcaacttacatttcaaacgctaacaaaattttaaaccgttcattcaacgtaatttcaacttaccacaaccacttaaaaacttgggaaaacaacccaaacccacaaaacactcaagacgtacgtactcaaattcaattagtacactaccacttccaaaacgtaattccagaattagtaaactcatgtccaccaaacccatcagactgtgactactacaacattttagtattatcatcatacgctcaagctgctaacttacacttaactgtattaaaccaagctgtaaaattcgaagcttacttaaaaaacaaccgtcaattcgactacttagaaccattaccaactgctattgactactacccagtattaactaaagctattgaagactacactaactactgtgtaactacttacaaaaaaggtttaaacttaattaaaactactccagactcaaacttagacggtaacattaactggaacacttacaacacttaccgtactaaaatgactactgctgtattagacttagtagctttattcccaaactacgacgtaggtaaatacccaattggtgtacaatcagaattaactcgtgaaatttaccaagtattaaacttcgaagaatcaccatacaaatactacgacttccaataccaagaagactcattaactcgtcgtccacacttattcacttggttagactcattaaacttctacgaaaaagctcaaactactccaaacaacttcttcacttcacactacaacatgttccactacactttagacaacatttcacaaaaatcatcagtattcggtaaccacaacgtaactgacaaattaaaatcattaggtttagctactaacatttacattttcttattaaacgtaatttcattagacaacaaatacttaaacgactacaacaacatttcaaaaatggacttcttcattactaacggtactcgtttattagaaaaagaattaactgctggttcaggtcaaattacttacgacgtaaacaaaaacattttcggtttaccaattttaaaacgtcgtgaaaaccaaggtaacccaactttattcccaacttacgacaactactcacacattttatcattcattaaatcattatcaattccagctacttacaaaactcaagtatacactttcgcttggactcactcatcagtagacccaaaaaacactatttacactcacttaactactcaaattccagctgtaaaagctaactcattaggtactgcttcaaaagtagtacaaggtccaggtcacactggtggtgacttaattgacttcaaagaccacttcaaaattacttgtcaacactcaaacttccaacaatcatacttcattcgtattcgttacgcttcaaacggttcagctaacactcgtgctgtaattaacttatcaattccaggtgtagctgaattaggtatggctttaaacccaactttctcaggtactgactacactaacttaaaatacaaagacttccaatacttagaattctcaaacgaagtaaaattcgctccaaaccaaaacatttcattagtattcaaccgttcagacgtatacactaacactactgtattaattgacaaaattgaattcttaccaattactcgttcaattcgtgaagaccgtgaaaaacaaaaattagaaactgtacaacaaattattaacactttcgactacaaagacgacgacgacaaataatctagtcgaggatcgatccggctgctaacaaagcccgaaaggaagctgagttggctgctgccaccgctgagctcaaacaactaatttttttttaaactaaaataaatctggttaaccatacctggtttattttagtttagtttatacacacttttcatatatatatacttaatagctaccataggcagttggcaggatcc

SEQ ID NO: 30 - DNA, 3883 bp, Artificial Sequence
  Synthetic
ggatcctcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttaaagatacaccatatgcaatcaggttacccattagctaacgacttacaaggttcaatgaaaaacactaactacaaagactggttagctatgtgtgaaaacaaccaacaatacggtgtaaacccagctgctattaactcatcatcagtatcaactgctttaaaagtagctggtgctattttaaaattcgtaaacccaccagctggtactgtattaactgtattatcagctgtattaccaattttatggccaactaacactccaactccagaacgtgtatggaacgacttcatgactaacactggtaacttaattgaccaaactgtaactgcttacgtacgtactgacgctaacgctaaaatgactgtagtaaaagactacttagaccaatacactactaaattcaacacttggaaacgtgaaccaaacaaccaatcataccgtactgctgtaattactcaattcaacttaacttcagctaaattacgtgaaactgctgtatacttctcaaacttagtaggttacgaattattattattaccaatttacgctcaagtagctaacttcaacttattattaattcgtgacggtttaattaacgctcaagaatggtcattagctcgttcagctggtgaccaattatacaacactatggtacaatacactaaagaatacattgctcactcaattacttggtacaacaaaggtttagacgtattacgtaacaaatcaaacggtcaatggattactttcaacgactacaaacgtgaaatgactattcaagtattagacattttagctttattcgcttcatacgacccacgtcgttacccagctgacaaaattgacaacactaaattatcaaaaactgaattcactcgtgaaatttacactgctttagtagaatcaccatcatcaaaatcaattgctgctttagaagctgctttaactcgtgacgtacacttattcacttggttaaaacgtgtagacttctggactaacactatttaccaagacttacgtttcttatcagctaacaaaattggtttctcatacactaactcatcagctatgcaagaatcaggtatttacggttcatcaggtttcggttcaaacttaactcaccaaattcaattaaactcaaacgtatacaaaacttcaattactgacacttcatcaccatcaaaccgtgtaactaaaatggacttctacaaaattgacggtactttagcttcatacaactcaaacattactccaactccagaaggtttacgtactactttcttcggtttctcaactaacgaaaacactccaaaccaaccaactgtaaacgactacactcacattttatcatacattaaaactgacgtaattgactacaactcaaaccgtgtatcattcgcttggactcacaaaattgtagacccaaacaaccaaatttacactgacgctattactcaagtaccagctgtaaaatcaaacttcttaaacgctactgctaaagtaattaaaggtccaggtcacactggtggtgacttagtagctttaacttcaaacggtactttatcaggtcgtatggaaattcaatgtaaaacttcaattttcaacgacccaactcgttcatacggtttacgtattcgttacgctgctaactcaccaattgtattaaacgtatcatacgtattacaaggtgtatcacgtggtactactatttcaactgaatcaactttctcacgtccaaacaacattattccaactgacttaaaatacgaagaattccgttacaaagacccattcgacgctattgtaccaatgcgtttatcatcaaaccaattaattactattgctattcaaccattaaacatgacttcaaacaaccaagtaattattgaccgtattgaaattattccaattactcaatcagtattagacgaaactgaaaaccaaaacttagaatcagaacgtgaagtagtaaacgctttattcactaacgacgctaaagacgctttaaacattggtactactgactacgacattgaccaagctgctaacttagtagaatgtatttcagaagaattatacccaaaagaaaaaatgttattattagacgaagtaaaaaacgctaaacaattatcacaatcacgtaacgtattacaaaacggtgacttcgaatcagctactttaggttggactacttcagacaacattactattcaagaagacgacccaattttcaaaggtcactacttacacatgtcaggtgctcgtgacattgacggtactattttcccaacttacattttccaaaaaattgacgaatcaaaattaaaaccatacactcgttacttagtacgtggtttcgtaggttcatcaaaagacgtagaattagtagtatcacgttacggtgaagaaattgacgctattatgaacgtaccagctgacttaaactacttatacccatcaactttcgactgtgaaggttcaaaccgttgtgaaacttcagctgtaccagctaacattggtaacacttcagacatgttatactcatgtcaatacgacactggtaaaaaacacgtagtatgtcaagactcacaccaattctcattcactattgacactggtgctttagacactaacgaaaacattggtgtatgggtaatgttcaaaatttcatcaccagacggttacgcttcattagacaacttagaagtaattgaagaaggtccaattgacggtgaagctttatcacgtgtaaaacacatggaaaaaaaatggaacgaccaaatggaagctaaacgttcagaaactcaacaagcttacgacgtagctaaacaagctattgacgctttattcactaacgtacaagacgaagctttacaattcgacactactttagctcaaattcaatacgctgaatacttagtacaatcaattccatacgtatacaacgactggttatcagacgtaccaggtatgaactacgacatttacgtagaattagacgctcgtgtagctcaagctcgttacttatacgacactcgtaacattattaaaaacggtgacttcactcaaggtgtaatgggttggcacgtaactggtaacgctgacgtacaacaaattgacggtgtatcagtattagtattatcaaactggtcagctggtgtatcacaaaacgtacacttacaacacaaccacggttacgtattacgtgtaattgctaaaaaagaaggtccaggtaacggttacgtaactttaatggactgtgaagaaaaccaagaaaaattaactttcacttcatgtgaagaaggttacattactaaaactgtagacgtattcccagacactgaccgtgtacgtattgaaattggtgaaactgaaggttcattctacattgaatcaattgaattaatttgtatgaacgaagactacaaagacgacgacgacaaataatctagtccggctgctaacaaagcccgaaaggaagctgagttggctgctgccaccgctgagctcaaacaactaatttttttttaaactaaaataaatctggttaaccatacctggtttattttagtttagtttatacacacttttcatatatatatacttaatagctaccataggcagttggcaggatcc